(19) United States
(12) Patent Application Publication
(10) Pub. No.: US 2025/0388607 A1
(43) Pub. Date: Dec. 25, 2025

(54) A BROMOTAG-BASED TOOLKIT FOR RAPID INDUCTION OF CHEMICALLY INDUCED PROXIMITY

(71) Applicant: THE BOARD OF TRUSTEES OF THE LELAND STANFORD JUNIOR UNIVERSITY, Stanford, CA (US)

(72) Inventors: Nathanael S. Gray, Stanford, CA (US); Steven Corsello, Stanford, CA (US); Muhammad Murtaza Hassan, Stanford, CA (US); Gianna Colombo, Stanford, CA (US)

(21) Appl. No.: 19/243,349

(22) Filed: Jun. 19, 2025

Related U.S. Application Data
(60) Provisional application No. 63/661,714, filed on Jun. 19, 2024.

(51) Int. Cl. C07D 519/00; C07D 495/14; C07K 14/47

(52) U.S. Cl. CPC C07D 519/00; C07D 495/14; C07K 14/4703

(57) ABSTRACT

Disclosed herein are compounds that stimulate formation of protein homodimers or heterodimers, which can be used for the study of protein-protein interactions.

### CROSS-REFERENCE TO RELATED APPLICATION

This application claims priority to and the benefit of U.S. Provisional Patent Application No. 63/661,714, filed on Jun. 19, 2024, the disclosure of which is incorporated herein by reference in its entirety.

### FIELD

Provided herein are compounds that stimulate formation of protein homodimers or heterodimers, which can be used for the study of protein-protein interactions.

### BACKGROUND

The induction of protein-protein interactions within cells is a valuable approach in cell and chemical biology research. A general method involves tagging proteins of interest with protein domains that can be recruited upon treatment with bivalent small molecules. Depending on the chemical ligand, protein homodimers or heterodimers can be stimulated within cells. Applications include alteration of signal transduction via chemically induced proximity and changing protein subcellular localization.

### SUMMARY

Disclosed herein is a series of bivalent molecules that rely on a “bump and hole” ligand to bind to the BRD4 BD2 L387A mutant tag (“BromoTag”). Compounds disclosed herein successfully stimulate formation of protein homodimers (dual BromoTag recruiters) or heterodimers (BromoTag-FKBP F36V recruiters). This system can be used for the study of protein-protein interactions.

In one aspect, disclosed herein is a compound of formula (I):

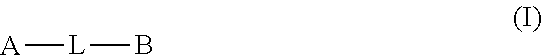

or a salt thereof, wherein:
A is a moiety that binds to BRD4 BD2-domain;
L is a linker; and
B is selected from: (i) a moiety that binds to BRD4 BD2-domain; and (ii) a moiety that binds to FKBP12.

In some embodiments, A is:

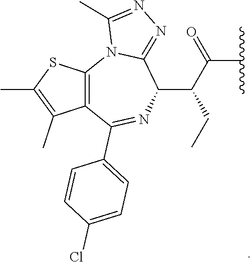 [SMILES: *C(=O)[C@H](CC)[C@@H]1N=C(c2ccc(Cl)cc2)c2c(sc(C)c2C)-n2c(C)nnc21]

In some embodiments, B is a moiety that binds to BRD4 BD2-domain. In some embodiments, B is:

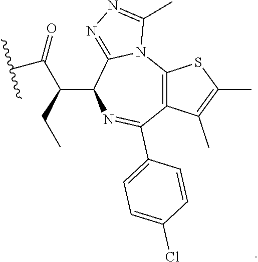 [SMILES: *C(=O)[C@H](CC)[C@@H]1N=C(c2ccc(Cl)cc2)c2c(sc(C)c2C)-n2c(C)nnc21]

In some embodiments, B is a moiety that binds to FKBP12. In some embodiments, B is:

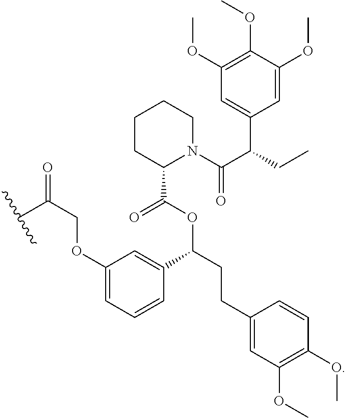 [SMILES: *C(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

In some embodiments, the compound is a compound of formula (Ia):

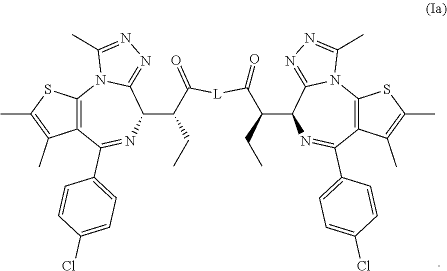

In some embodiments, the compound is a compound of formula (Ib):

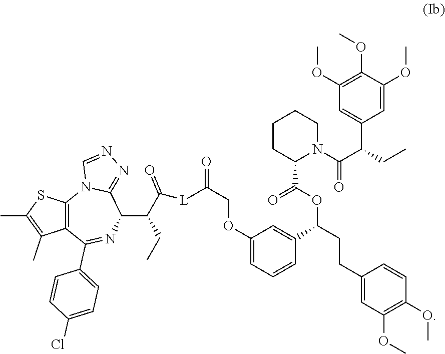

In some embodiments, L is a direct bond or comprises any combination of —CH₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene moieties, wherein the arylene, heteroarylene, cycloalkylene, and heterocyclylene moieties are independently unsubstituted or substituted with 1, 2, or 3 substituents. In some embodiments, L is a direct bond or comprises any combination of —CH₂—, —O—, —NH—, and heterocyclylene moieties. In some embodiments, L comprises any combination of the following moieties:

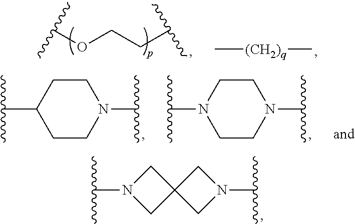

wherein p is 1, 2, 3, or 4; and q is 1, 2, 3, 4, 5, 6, 7, 8, 9, 10, 11, or 12.

In some embodiments, L is:

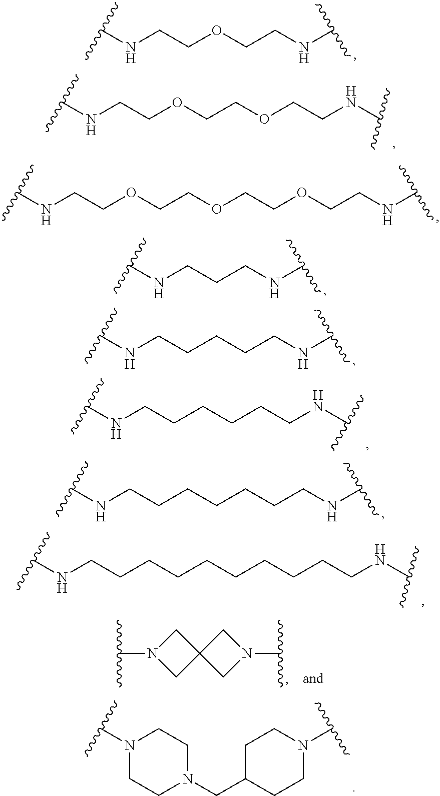

In some embodiments, the compound is selected from:

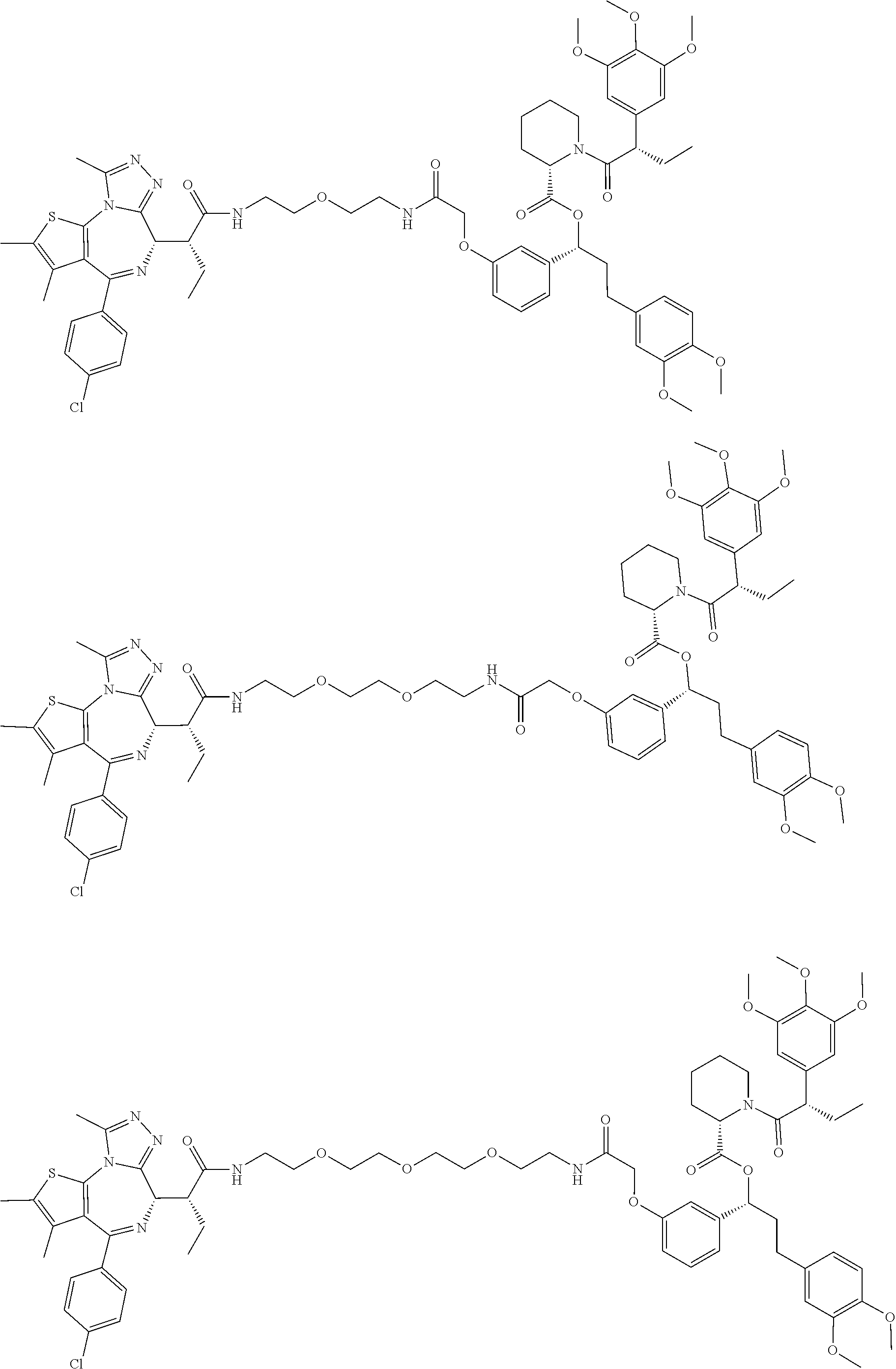

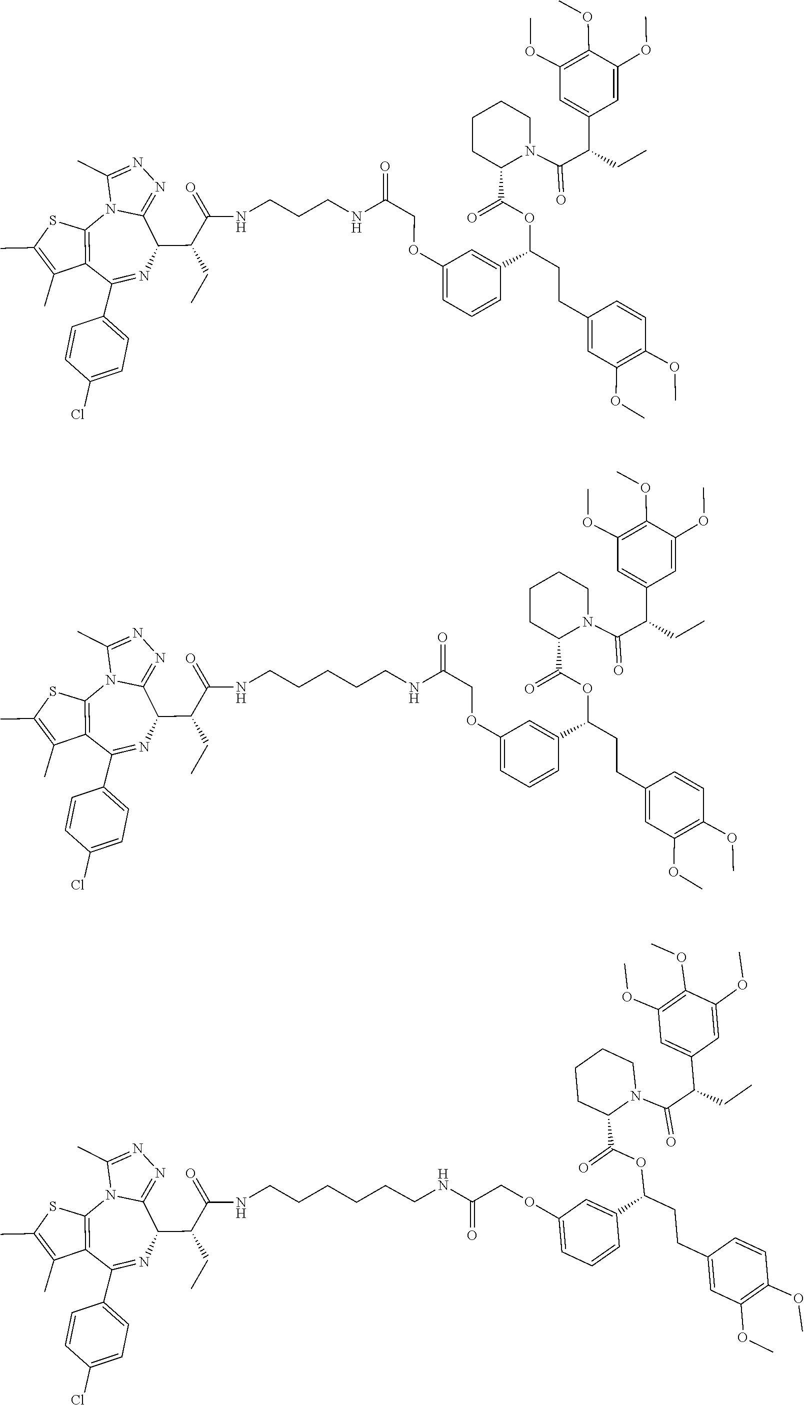

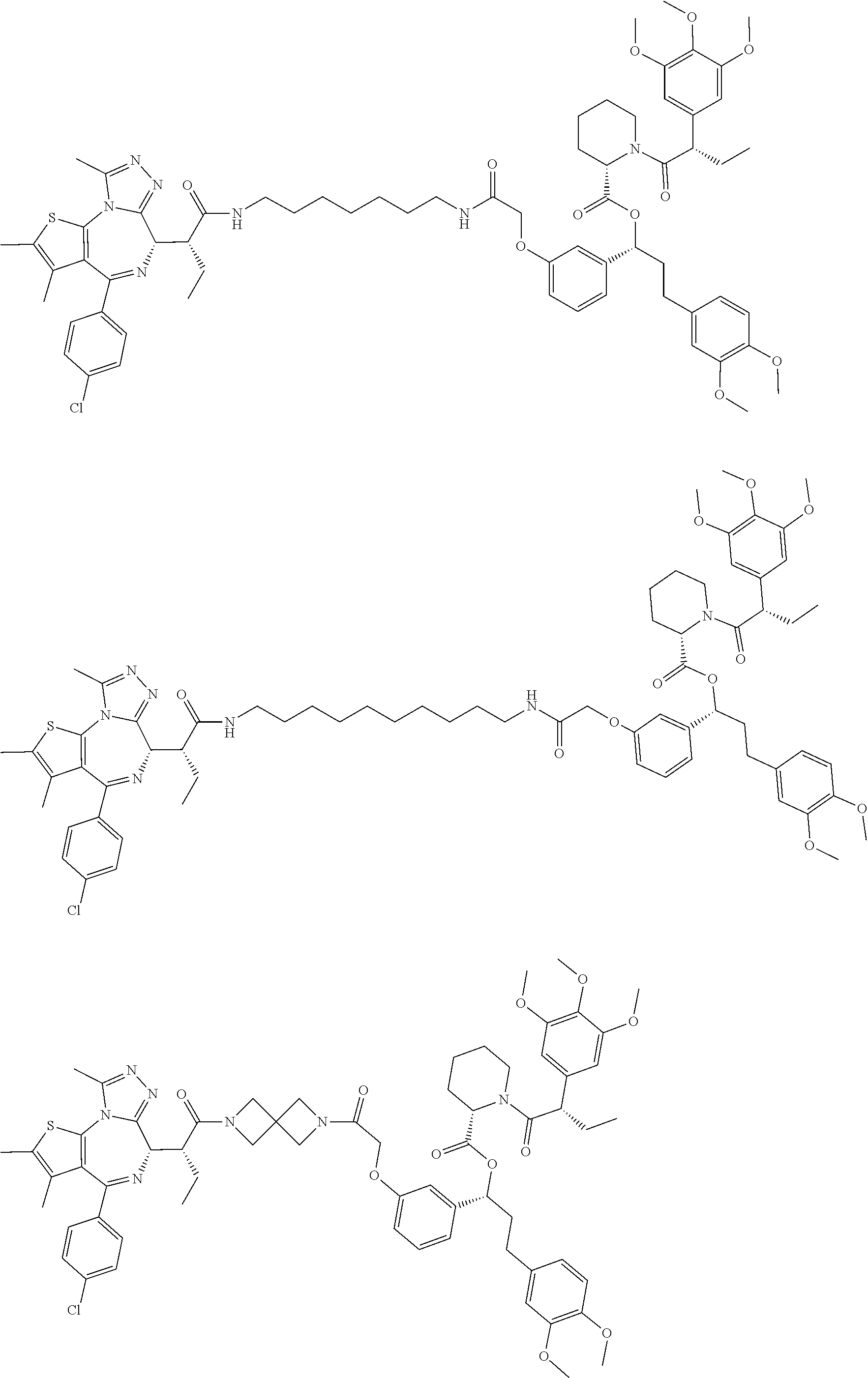

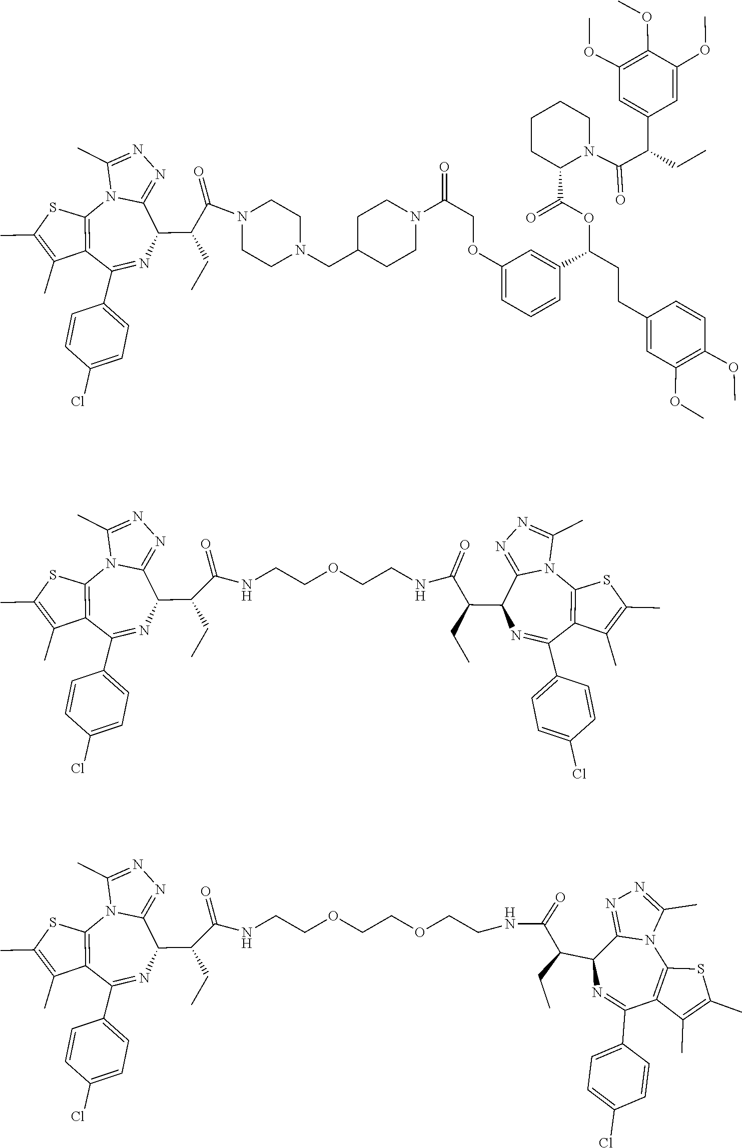

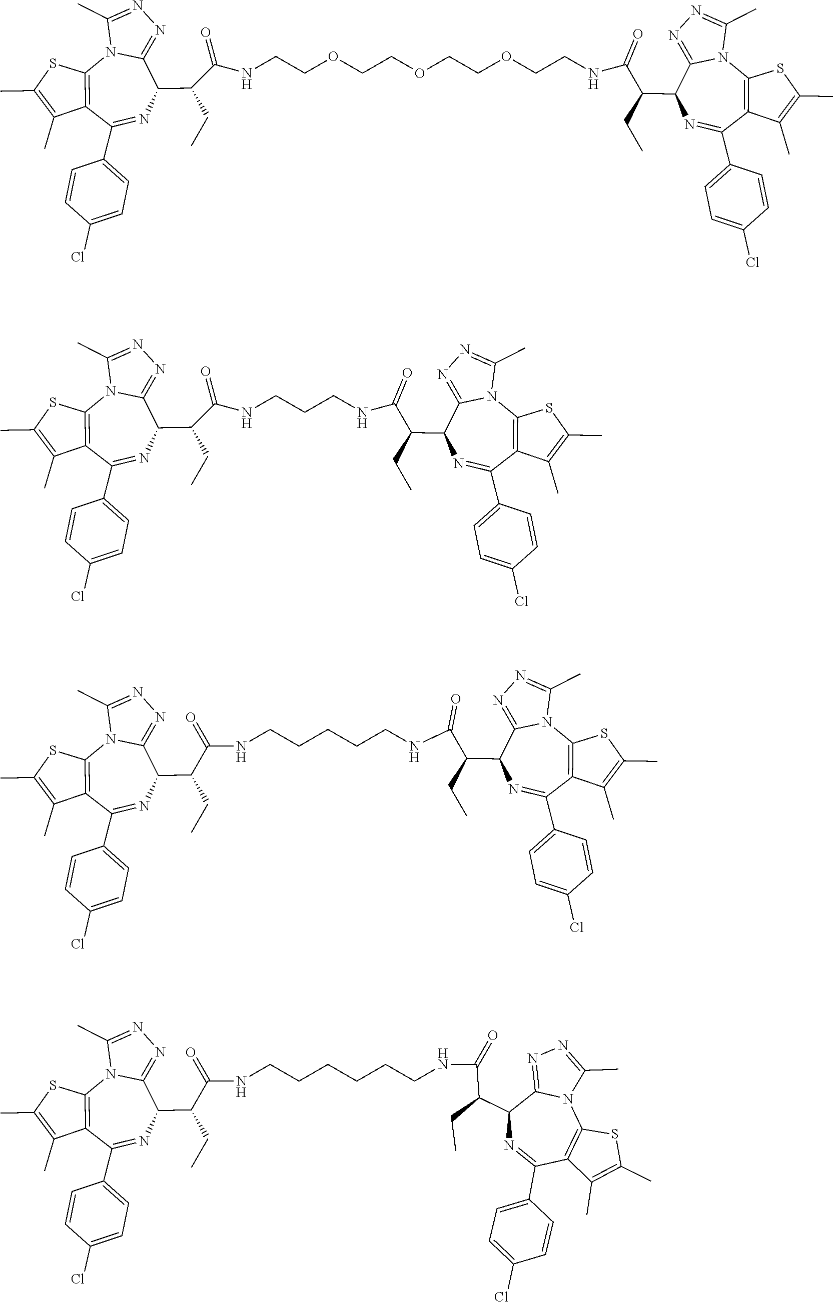

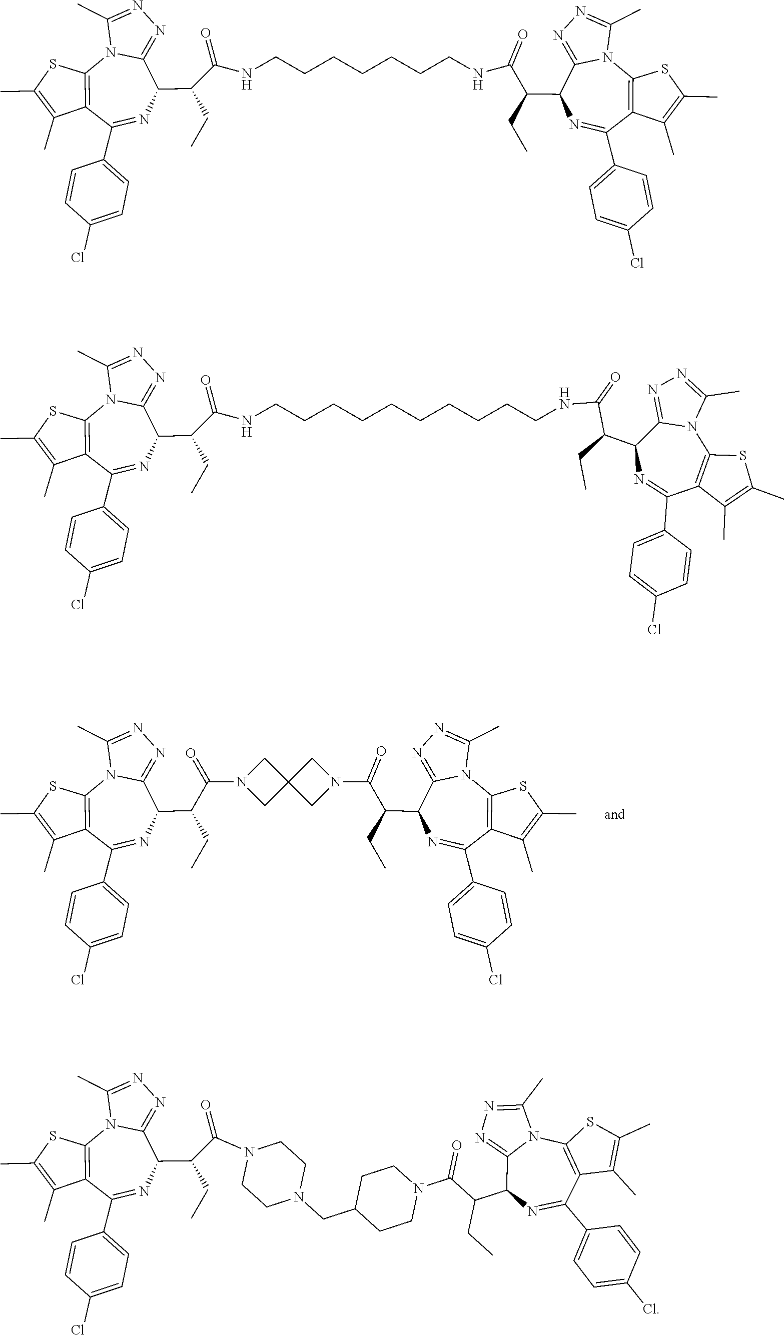

### BRIEF DESCRIPTION OF THE DRAWINGS

FIGS. 1A-1D show results of NanoBiT assay dimerization testing of MMH-04-74 FKBP/BromoTag bivalent heterodimerizer compound in all possible orientations of LgBiT and SmBiT tags. For this assay, FKBP/BromoTag/FRB were expressed alone, with no fusion protein partners.

FIGS. 2A-2B show results of NanoBiT assay dimerization testing for library of ten FKBP/BromoTag bivalent heterodimerizer compounds in optimal Nterm-LgBiT/Nterm-SmBiT tag orientation (observed in FIG. 1). FKBP/FRB-sirolimus control system is boxed for comparison. For this assay, FKBP/BromoTag/FRB were expressed alone, with no fusion protein partners.

FIGS. 3A-3B show quantification of NanoBiT assay dimerization testing for library of ten FKBP/BromoTag bivalent heterodimerizer compounds. For this assay, FKBP/BromoTag/FRB were expressed alone, with no fusion protein partners.

FIGS. 4A-4D show results of NanoBiT assay dimerization testing of MMH-05-01-02 and MMH-05-01-03 (later renamed MMH-05-08-2) BromoTag bivalent homodimerizer compounds in all possible orientations of LgBiT and SmBiT tags. For this assay, FKBP/BromoTag/FRB were expressed alone, with no fusion protein partners.

FIG. 5 shows quantification of NanoBiT assay dimerization testing for MMH-05-01-02 and MMH-05-01-03 (later renamed MMH-05-08-2) bivalent homodimerizer compounds in all possible orientations of LgBiT and SmBiT tags. For this assay, BromoTag was expressed alone, with no fusion protein partner.

FIGS. 6A-6B show quantification of NanoBiT assay dimerization testing for a library of ten BromoTag homodimerizers and ten FKBP/BromoTag heterodimerizers. For this assay, FKBP and BromoTag were appended to Casp9 protein.

FIG. 7 shows dose optimization of a BromoTag homodimerizer (MMH-05-08-3) and a FKBP/BromoTag heterodimerizer (MMH-05-07-3).

FIGS. 8A-8B show quantification of the activation of Casp9, a pro-apoptotic protein, via chemically-induced dimerization (CID) using cell death assays.

FIG. 9 shows Western blots depicting c-KIT activation (quantified by c-KIT phosphorylation at residue Y703) following CID.

FIG. 10 shows Western blots depicting c-KIT activation following CID, testing the effect of extending the protein linker between BromoTag and c-KIT or including multiple BromoTags in tandem.

FIGS. 11A-11C show data for a Ba/F3 c-KIT cytokine independence growth assay.

FIGS. 12A-12C show quantification of NanoBiT assay dimerization testing for a library of ten BromoTag bivalent homodimerizers. Blue asterisks indicate high background signal. For this assay, FKBP/BromoTag were expressed alone, with no fusion protein partners.

### DETAILED DESCRIPTION

Provided herein are compounds that stimulate formation of protein homodimers or heterodimers, which can be used for the study of protein-protein interactions. This system should be useful for facilitating the study of protein-protein interactions.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a”, “an”, and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-C₈ alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “amino” refers to a group —NR₂, wherein each R is independently selected from hydrogen and alkyl (e.g., C₁-C₄ alkyl). A group —NH(alkyl) may be referred to herein as “alkylamino” and a group —N(alkyl)₂ may be referred to herein as “dialkylamino.”

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl,” i.e., phenyl). In some embodiments, an aryl group has ten ring carbon atoms (“C₁₀ aryl,” e.g., naphthyl such as 1-naphthyl and 2-naphthyl). In some embodiments, an aryl group has fourteen ring carbon atoms (“C₁₄ aryl,” e.g., anthracenyl and phenanthrenyl).

As used herein, the term “arylene” refers to a divalent aryl radical.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “cycloalkylene” refers to a divalent cycloalkyl radical.

As used herein, the term “cyano” refers to a —CN group.

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen (“perhaloalkyl”). Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “haloalkoxy” refers to a haloalkyl group, as defined herein, appended to the parent molecular moiety through an oxygen atom. Representative examples of haloalkoxy include, but are not limited to, difluoromethoxy, trifluoromethoxy, and 2,2,2-trifluoroethoxy.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heteroarylene” refers to a divalent heteroaryl radical.

As used herein, the term “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “heterocyclylene” refers to a divalent heterocyclyl radical.

As used herein, the term “hydroxy” or “hydroxyl” refers to an —OH group.

As used herein, the term “nitro” refers to an —NO₂ group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroaryl, heterocyclyl, hydroxy, hydrazino, imino, oxo, nitro, phosphate, phosphonate, sulfonic acid, thiol, thione, or combinations thereof.

As used herein, in chemical structures the indication:

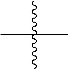

represents a point of attachment of one moiety to another moiety (e.g., a substituent group to the rest of the compound).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

When substituent groups are specified by their conventional chemical formulae, written from left to right, such indication also encompass substituent groups resulting from writing the structure from right to left. For example, if a bivalent group is shown as —CH₂O—, such indication also encompasses —OCH₂—; similarly, —OC(O)NH— also encompasses —NHC(O)O—. When linker moieties are shown, the linkers can be attached to other moieties of the compound in either direction.

### Compounds

Disclosed herein are compounds of formula (I):

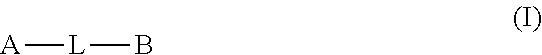

and salts thereof, wherein:
A is a moiety that binds to BRD4 BD2-domain;
L is a linker; and
B is selected from: (i) a moiety that binds to BRD4 BD2-domain; and (ii) a moiety that binds to FKBP12.

In some embodiments, A is:

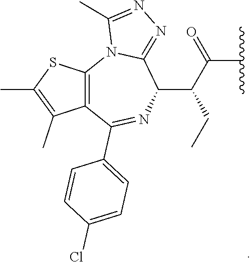 [SMILES: *C(=O)[C@H](CC)[C@@H]1N=C(c2ccc(Cl)cc2)c2c(sc(C)c2C)-n2c(C)nnc21]

In some embodiments, B is a moiety that binds to BRD4 BD2-domain. In some embodiments, B is:

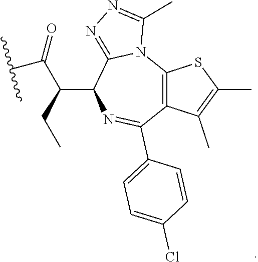 [SMILES: *C(=O)[C@H](CC)[C@@H]1N=C(c2ccc(Cl)cc2)c2c(sc(C)c2C)-n2c(C)nnc21]

In some embodiments, B is a moiety that binds to FKBP12. In some embodiments, B is:

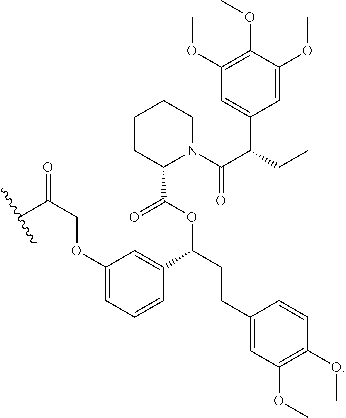 [SMILES: *C(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

In some embodiments, the compound of formula (I) is a compound of formula (Ia):

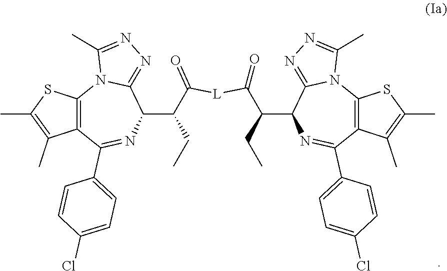

and salts thereof.

In some embodiments, the compound of formula (I) is a compound of formula (Ib):

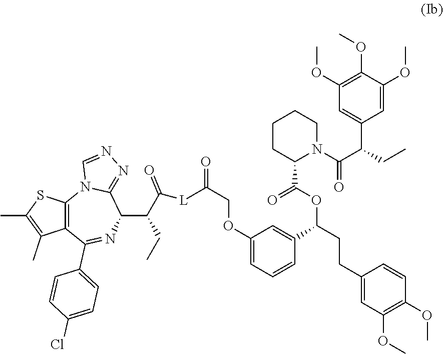

and salts thereof.

In some embodiments, L is a direct bond or comprises any combination of —CH₂—, —CH═CH—, —C≡C—, —O—, —NR′—, —BR′—, —S—, —C(O)—, —C(NR′)—, —S(O)—, —S(O)₂—, arylene, heteroarylene, cycloalkylene, and heterocyclylene moieties, wherein the arylene, heteroarylene, cycloalkylene, and heterocyclylene moieties are independently unsubstituted or substituted with 1, 2, or 3 substituents. In some embodiments, L is a direct bond or comprises any combination of —CH₂—, —O—, —NH—, and heterocyclylene moieties. In some embodiments, L comprises any combination of the following moieties:

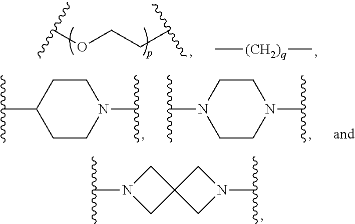

wherein p is 1, 2, 3, or 4; and q is 1, 2, 3, 4, 5, 6, 7, 8, 9, 10, 11, or 12.

In some embodiments, L has a formula selected from:

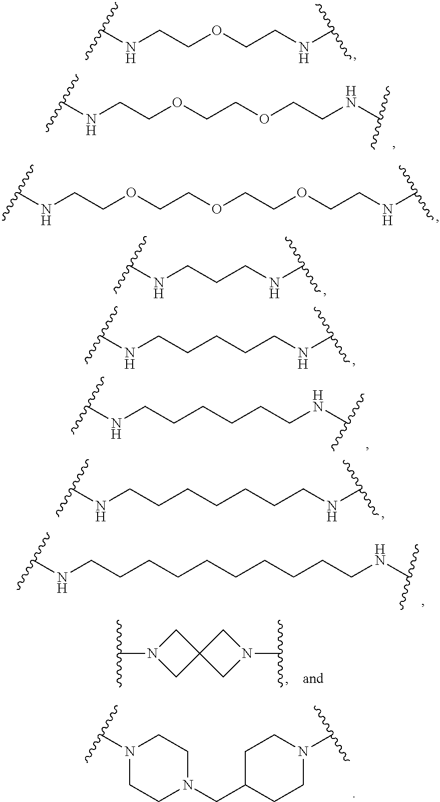

In some embodiments, the compound is selected from:

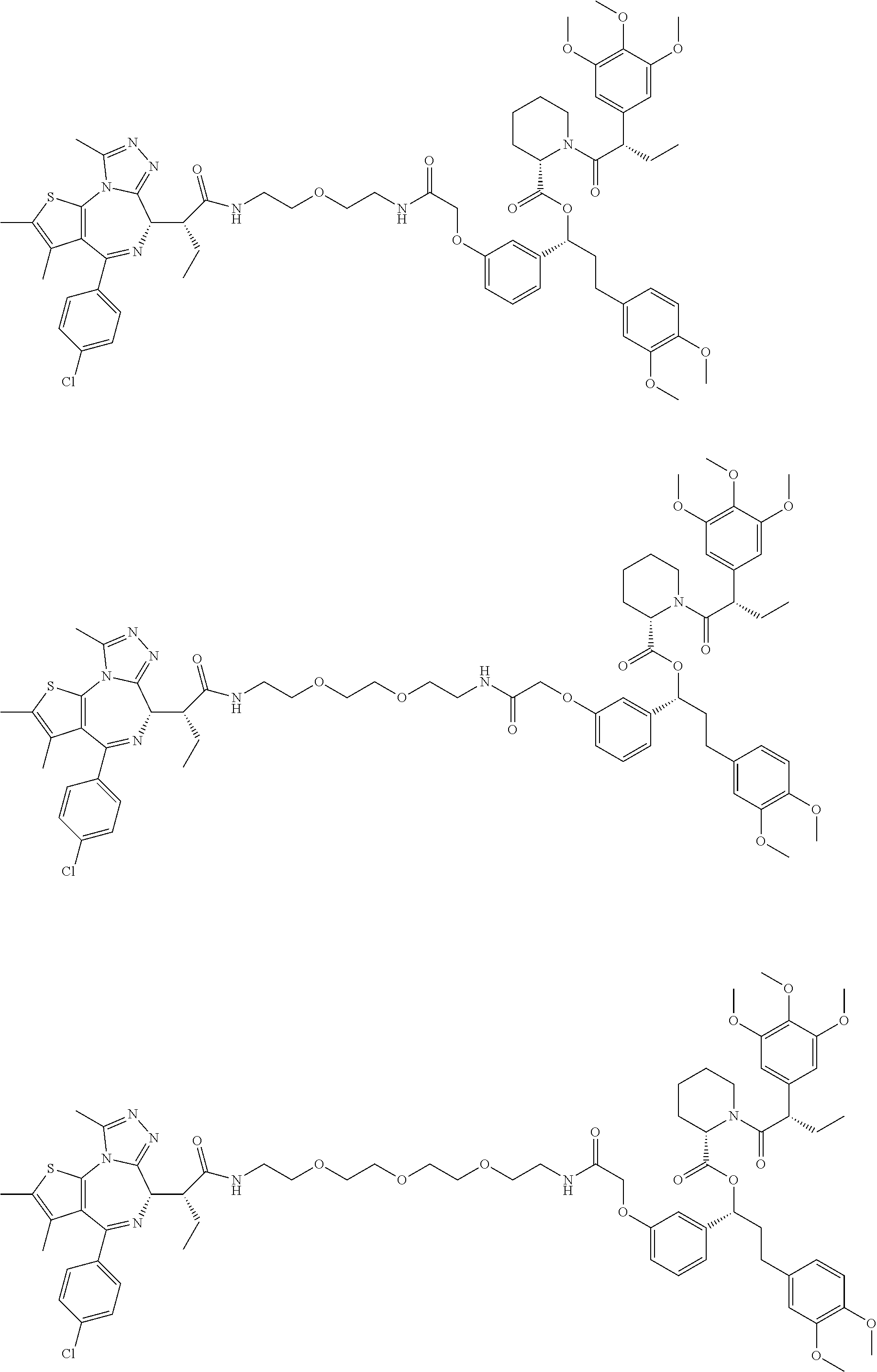

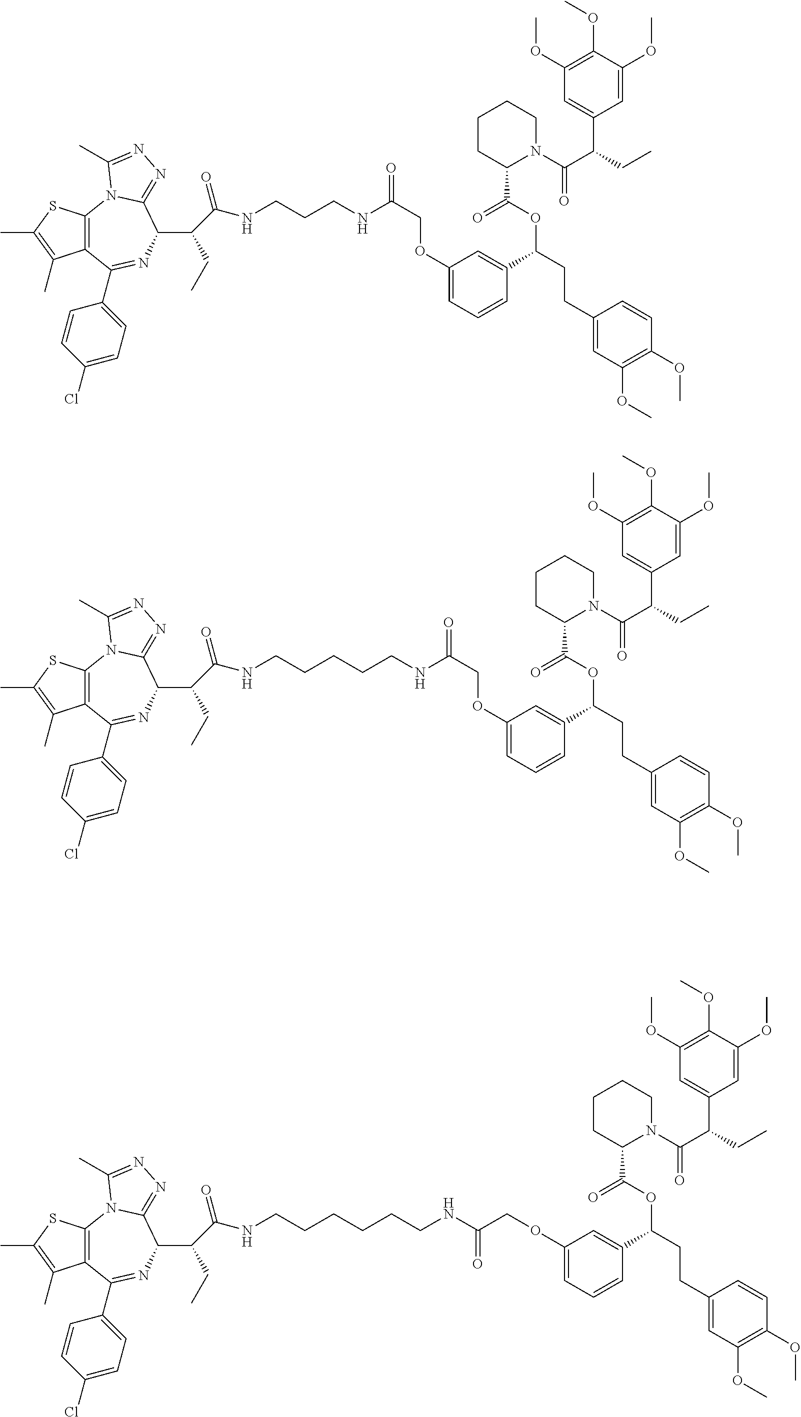 [SMILES: CC[C@H](C(=O)N1CCCC[C@H]1C(=O)O[C@H](CCc1ccc(OC)c(OC)c1)c1cccc(OCC(=O)NCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1]

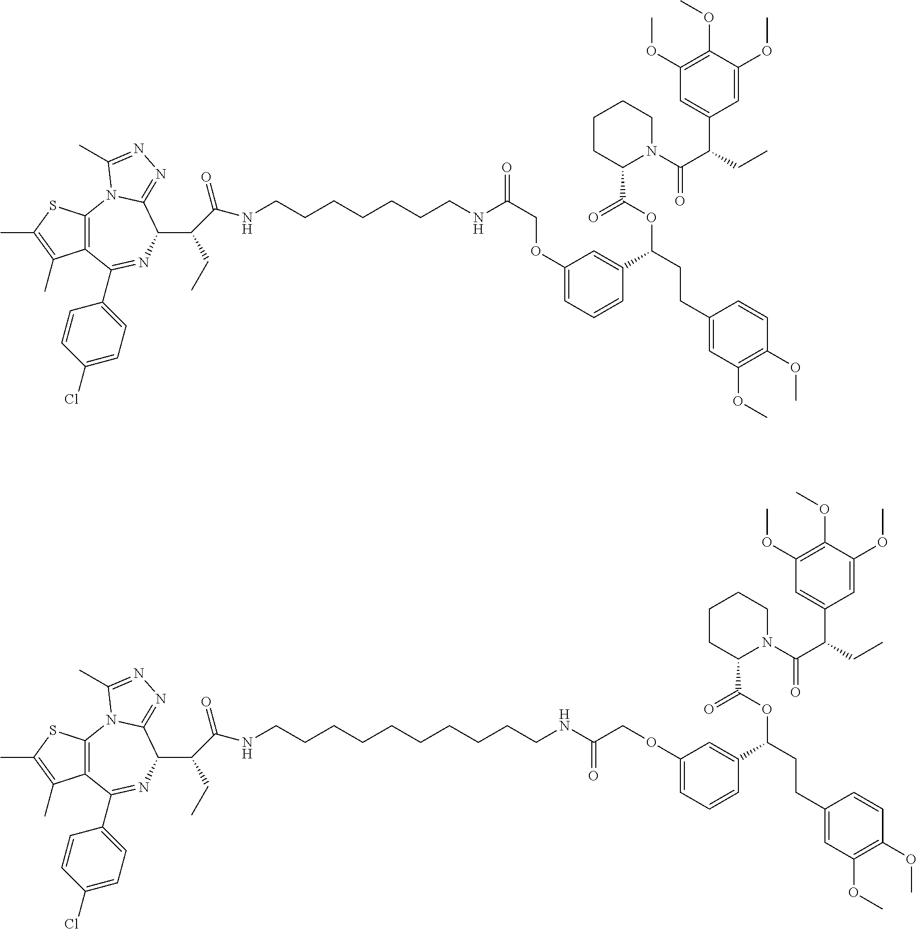 [SMILES: CC[C@H](C(=O)N1CCCC[C@H]1C(=O)O[C@H](CCc1ccc(OC)c(OC)c1)c1cccc(OCC(=O)NCCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1]

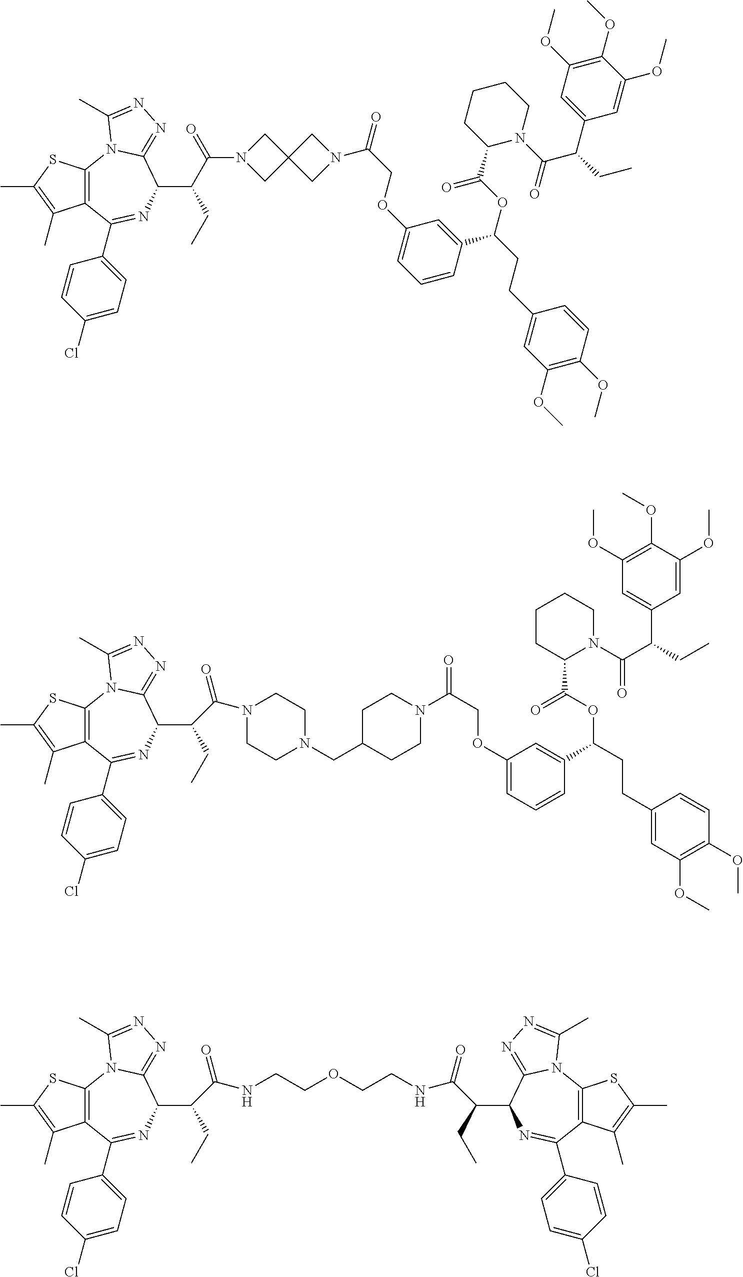

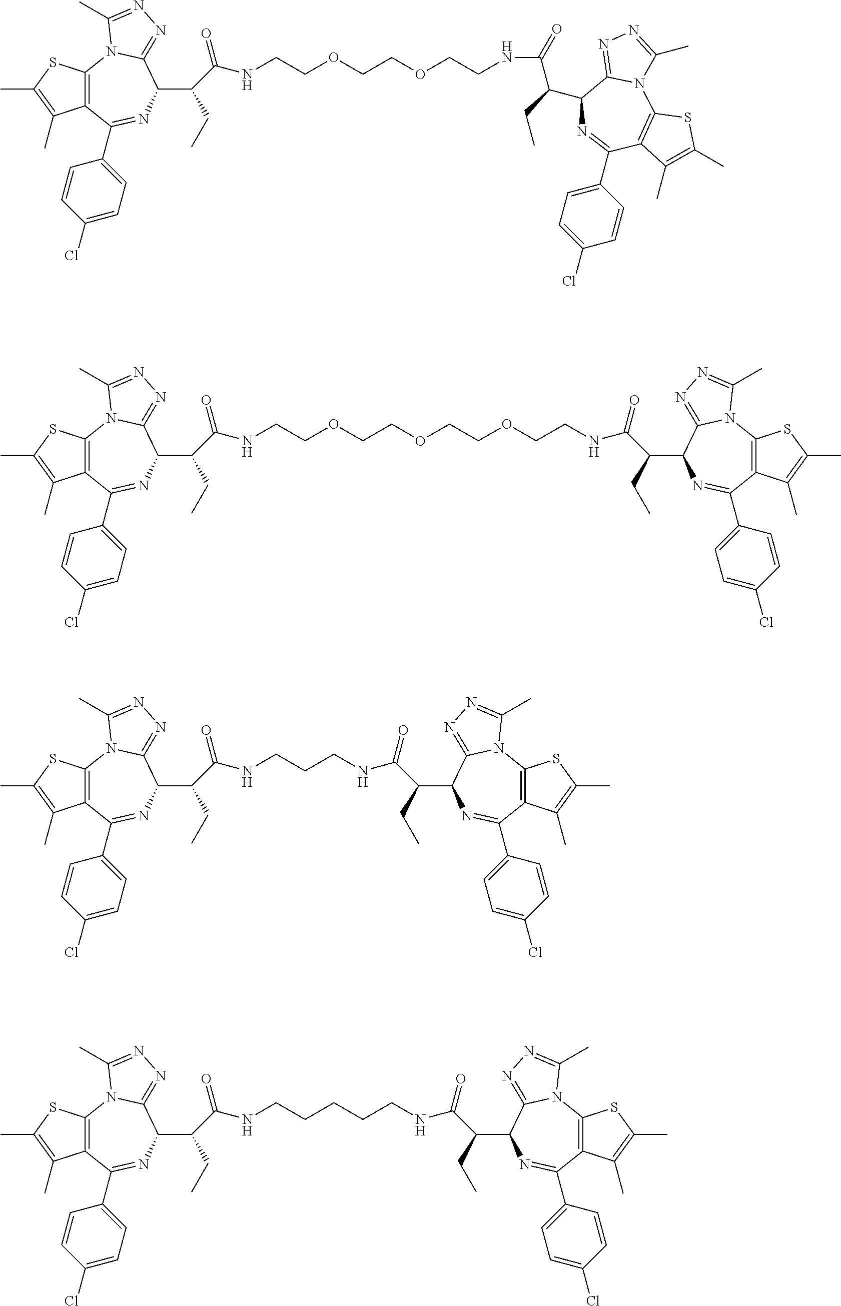

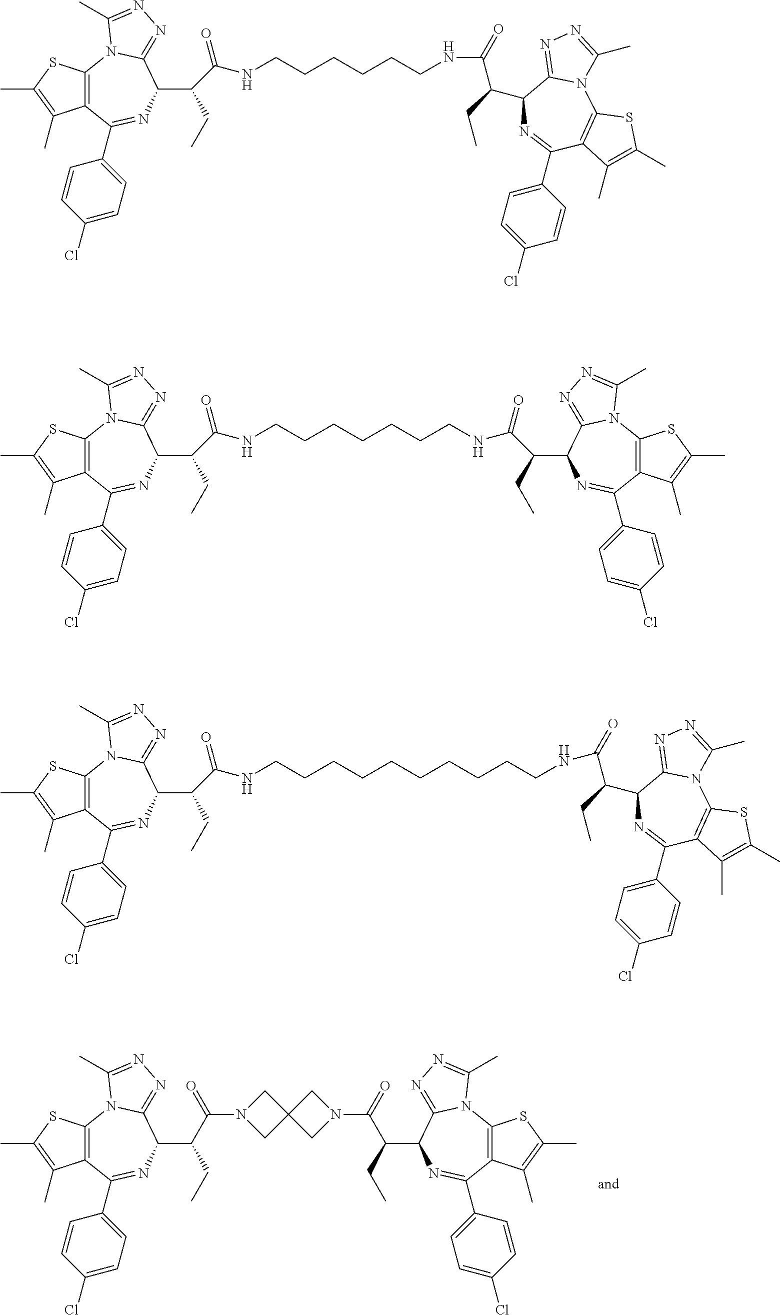

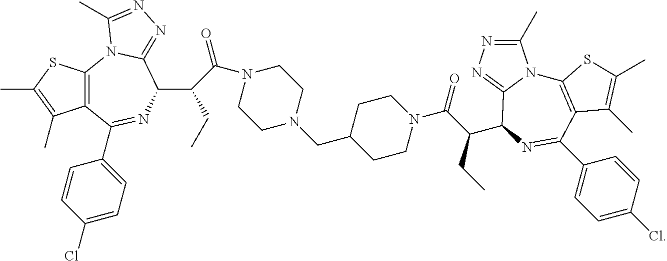 [SMILES: CC[C@@H](C(=O)N1CCC(CN2CCN(C(=O)[C@H](CC)[C@@H]3N=C(c4ccc(Cl)cc4)c4c(sc(C)c4C)-n4c(C)nnc43)CC2)CC1)[C@@H]1N=C(c2ccc(Cl)cc2)c2c(sc(C)c2C)-n2c(C)nnc21]

When discussing certain features or properties of compounds of formula (I) herein, or compositions, methods, or kits comprising compounds of formula (I), it is understood that such reference also includes compounds of formula (Ia), formula (Ib), and to specific exemplary compounds disclosed herein.

Certain compounds described herein may have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers, in mixtures and as pure or partially purified compounds, are included within the scope of this disclosure.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art. Alternatively, any enantiomer of a compound may be obtained by stereoselective synthesis using optically pure starting materials or reagents of known configuration by methods well known in the art.

Compounds may also possess tautomeric forms, and all tautomers also constitute embodiments of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium (²H) can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

Compounds disclosed herein can exist in solvated as well as unsolvated forms with pharmaceutically acceptable solvents such as water, ethanol, and the like, and it is intended that the disclosure encompass both solvated and unsolvated forms. In one embodiment, the compound is amorphous. In one embodiment, the compound is a single polymorph. In another embodiment, the compound is a mixture of polymorphs. In another embodiment, the compound is in a crystalline form.

a. Methods of Synthesis

Compounds disclosed herein can be prepared by a variety of methods, including those illustrated in the Examples. An exemplary synthesis is illustrated in Scheme 1.

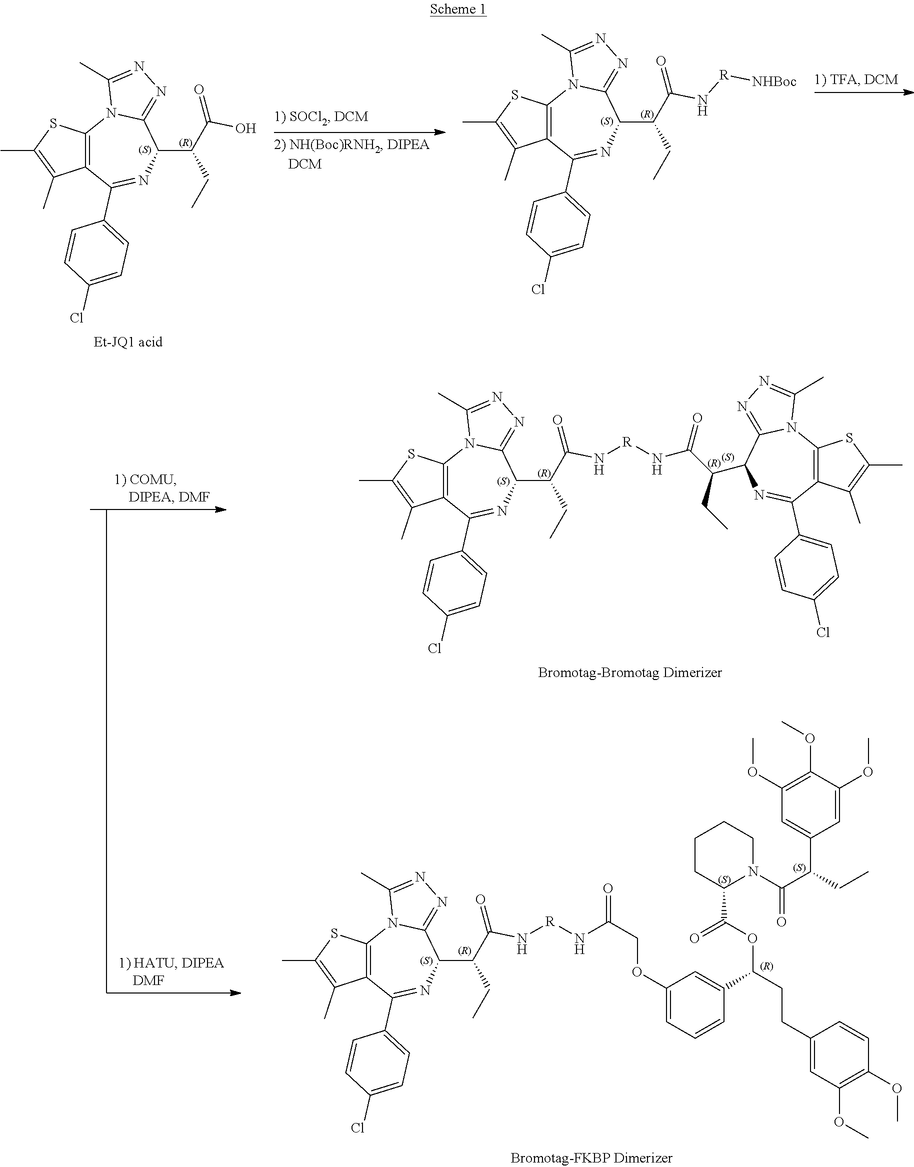

Compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples as described are illustrative and are not to be read as limiting the scope of the disclosure or the claims. Alternatives, modifications, and equivalents of the synthetic methods and specific examples are contemplated.

The disclosed compounds may exist as salts. The salts may be prepared during the final isolation and purification of the compounds or separately by reacting an amino group of the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. Amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Methods of Use and Kits

Compounds disclosed herein should find use for stimulating formation of protein homodimers or heterodimers, which may alter signal transduction in cells via chemically induced proximity, and may change protein subcellular localization. Such methods should be useful for the study of protein-protein interactions.

Accordingly, in some embodiments, disclosed herein is a method of stimulating formation of protein homodimers. In some embodiments, the method comprises stimulating the formation of protein homodimers by inducing dimerization between two Brd4 BD2 domains. To that end, in some embodiments, disclosed herein is a method of inducing dimerization between two Brd4 BD2 domains in a sample, comprising contacting the sample with a compound disclosed herein (e.g., a compound of formula (I)), or a salt thereof. In some embodiments, the compound disclosed herein is a dual BromoTag recruiter. In some embodiments, disclosed herein is a method of inducing dimerization between two Brd4 BD2 domains in a sample, comprising contacting the sample with a compound of formula (Ia), or a salt thereof.

In some embodiments, disclosed herein is a method of stimulating formation of protein heterodimers. In some embodiments, the method comprises stimulation the formation of protein heterodimers by inducing dimerization between a Brd4 BD2 domain and another domain (e.g., FKBP12). To that end, in some embodiments, disclosed herein is a method of inducing dimerization between a Brd4 BD2 domain and another domain (e.g., FKBP12), comprising contacting a sample with a compound disclosed herein (e.g., a compound of formula (I)), or a salt thereof. In some embodiments, the compound disclosed herein is a BromoTag-FKBP F36V recruiter. In some embodiments, disclosed herein is a method of inducing dimerization between a Brd4 BD2 domain and FKBP12 in a sample, comprising contacting a sample with a compound disclosed herein (e.g., a compound of formula (I)), or a salt thereof. In some embodiments, disclosed herein is a method of inducing dimerization between a Brd4 BD2 domain and FKBP12 in a sample, comprising contacting the sample with a compound of formula (Ib), or a salt thereof.

The present disclosure further provides a system or kit comprising a compound described herein (e.g., a compound of formula (I)), or a salt thereof. In some embodiments, the system or kit comprises a compound that induces dimerization between two Brd4 BD2 domains (e.g., a dual BromoTag recruiter). To that end, in some embodiments, the system or kit comprises a compound of formula (Ia), or a salt thereof. In some embodiments, the system or kit comprises a compound that induces dimerization between a Brd4 BD2 domain and another domain (e.g., FKBP12). In some embodiments, the system or kit comprises a compound that induces dimerization between a Brd4 BD2 domain and FKBP12. In some embodiments, the system or kit comprises a compound of formula (Ib), or a salt thereof.

The system or kit includes the compound, either alone or in a solvent (e.g., DMSO). In some embodiments, the system or kit may further comprise one or more additional reagents for conducting an assay to detect a protein-protein interaction. For example, the kit may further comprise one or more additional reagents necessary for the completion of a protein complementation assay or resonance transfer assay.

Systems or kits may further include a carrier or package such as a box, carton, tube or the like, having therein one or more containers, such as vials, tubes, ampoules, or bottles, which contain a compound of formula (I) or a salt thereof, or a composition comprising a compound of formula (I) or a salt thereof. Systems or kits may also include printed instructions for using the compounds, e.g., in an assay to detect protein-protein interactions.

### EXAMPLES

### Example 1

### Compound Syntheses

General experimental. Unless otherwise noted, all solvents and reagents were obtained from commercial vendors such as Sigma Aldrich, Oakwood Chemicals, Fischer Scientific, Combi Blocks, TCI Chemicals, and Alfa Aesar. Anhydrous solvents were used from Sure Seal bottles and were used without further purification. All reactions were conducted in scintillation vials and were monitored with LCMS. All final compounds were purified by HPLC, and their purity was determined by the LC trace.

Analytical Conditions and Instrumentation. All NMR spectra were obtained on a 500 MHz Bruker Avance III spectrometer or on 500 MHz Bruker Avance Neo equipped with carbon detect, liquid nitrogen cooled, Prodigy cryoprobe. All deuterated solvents were purchased from either Sigma Aldrich or Cambridge isotope laboratories. Coupling constants (J) are reported in Hertz (Hz) and multiplicities are reported as singlet (s), doublet (d), triplet (t), quartet (q), pentet (p), sextet (sex), septet (sep), multiplet (m), and broad (br), and the combination of these are listed as a combination of their abbreviations.

LCMS was conducted on an Acquity I-Class LCMS system equipped with a PDA-UV system, a QDa mass detector, and a dual column set-up (BEH and CSH columns). The solvent gradient consisted of LCMS grade acetonitrile purchased from Fischer Scientific, and milliQ water. A gradient of 15 to 100% acetonitrile in milliQ water, over 2.5 minutes followed by a 0.5 minute flush with 100% acetonitrile, was used for the final compounds described herein. All LCMS solvents contained 0.1% LCMS grade formic acid.

Preparative HPLC was used to purify all final compounds unless otherwise stated. HPLC grade methanol purchased from Fischer Scientific, and milliQ water, both containing 0.045% trifluoroacetic acid (TFA) were used for the solvent system. A 254 nm UV-light absorption was used to visualize compound chromatograms. The following method was used for the solvent gradient system:

Method 1: Waters Sunfire C18 column (30 mm×250 mm, 5 μm) using a gradient of 50 to 100% methanol in water containing 0.045% TFA (unless otherwise specified) over 20 min followed by a 5 min flush with 100% methanol, at a flow rate of 40 mL/min.

Abbreviations. Et, ethyl; DMSO, dimethyl sulfoxide; HATU, hexafluorophosphate azabenzotriazole tetramethyl uranium; COMU, 1-cyano-2-ethoxy-2-oxoethylidenaminooxy)dimethylamino-morpholino-carbenium hexafluorophosphate; DMF, dimethyl formamide; TFA, trifluoroacetic acid; DCM, dichloromethane; DIPEA, diisopropylethylamine; prep, preparative; HPLC, high performance liquid chromatography; LC, liquid chromatography; MS, mass spectrometry.

### Example 1

### Compound Syntheses

### Synthetic Experimental

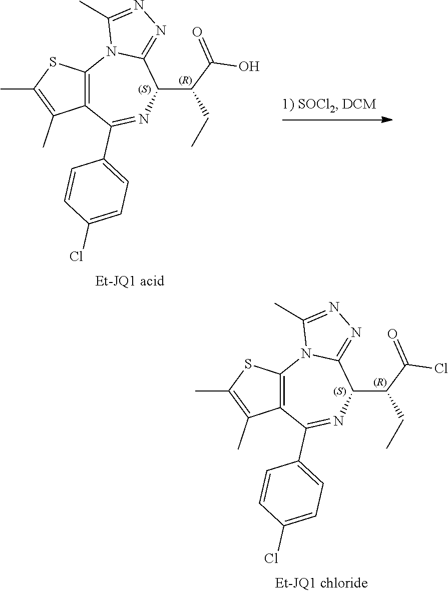

(R)-2-((S)-4-(4-chlorophenyl)-2,3,9-trimethyl-6H-thieno[3,2-f][1,2,4]triazolo[4,3-a][1,4]diazepin-6-yl)butanoyl chloride (Et-JQ1 chloride). EtJQ1 acid (151 mg, 0.352 mmol), obtained commercially from WuXi LabNetwork, was dissolved in 8 mL DCM and 1.1 mL thionyl chloride (17.6 mmol) was added subsequently. The reaction was left to stir overnight. The reaction progress was monitored via LCMS (˜2 uL reaction aliquots diluted in methanol) the consumption of the Et-JQ1 acid peak and the appearance of the methyl ester peak. After the complete conversion to Et-JQ1 chloride, the reaction mixture was thoroughly dried under reduced pressure. MS (ESI) for C₂₂H₂₄ClN₄O₂S [M+H]⁺: m/z calcd, 443.12; found, 443.20.

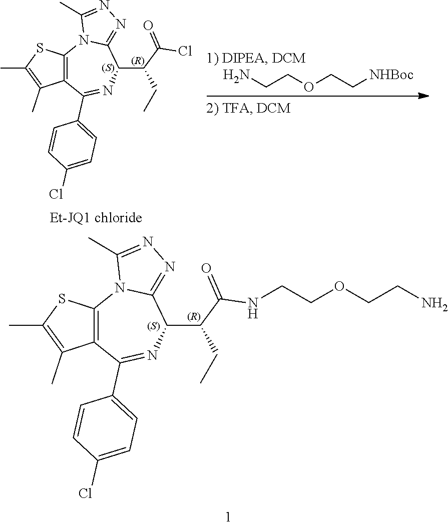

(R)-N-(2-(2-aminoethoxy)ethyl)-2-((S)-4-(4-chlorophenyl)-2,3,9-trimethyl-6H-thieno[3,2-f][1,2,4]triazolo[4,3-a][1,4]diazepin-6-yl)butanamide (1). To a vial containing NHBoc-PEG1-amine (7.99 mg, 0.03912 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (26.0 mg, 0.04303 mmol). MS (ESI) for C₃₀H₄₀ClN₆O4S [M+H]⁺: m/z calcd, 615.24; found, 615.34. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₅H₃₂ClN₆O₂S [M+H]⁺: m/z calcd, 515.19; found, 515.17.

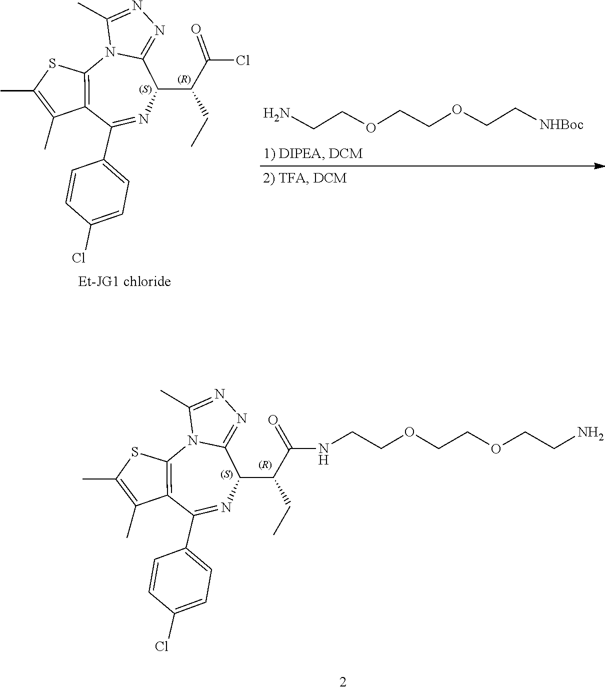

(R)-N-(2-(2-(2-aminoethoxy)ethoxy)ethyl)-2-((S)-4-(4-chlorophenyl)-2,3,9-trimethyl-6H-thieno[3,2-f][1,2,4]triazolo[4,3-a][1,4]diazepin-6-yl)butanamide (2). To a vial containing NHBoc-PEG2-amine (12.84 mg, 0.05172 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (25.8 mg, 0.03912 mmol). MS (ESI) for C₃₂H₄₄ClN₆O₅S [M+H]⁺: m/z calcd, 659.27; found, 659.40. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₇H₃₆ClN₆O₃S [M+H]⁺: m/z calcd, 559.22; found, 559.23.

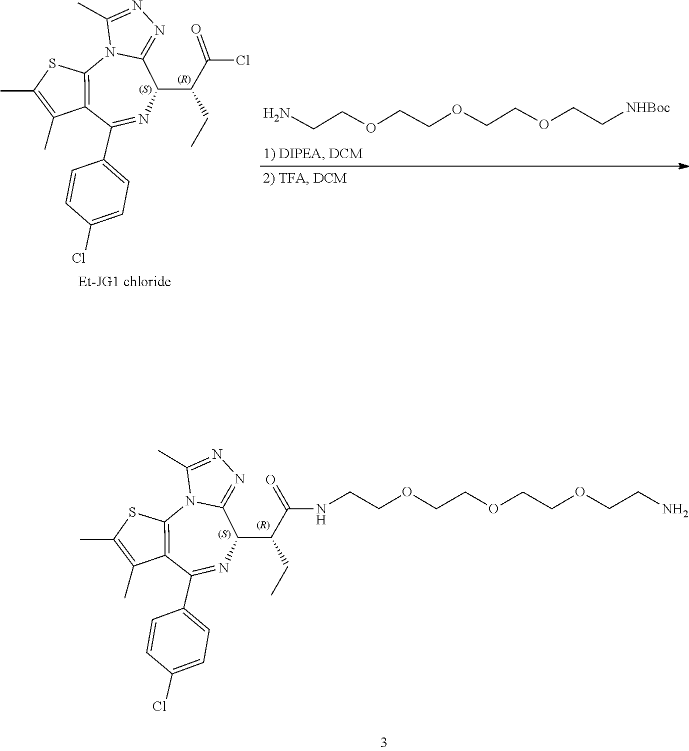

(R)-N-(2-(2-(2-(2-aminoethoxy)ethoxy)ethoxy)ethyl)-2-((S)-4-(4-chlorophenyl)-2,3,9-trimethyl-6H-thieno[3,2-f][1,2,4]triazolo[4,3-a][1,4]diazepin-6-yl)butanamide (3). To a vial containing NHBoc-PEG3-amine (17.62 mg, 0.06027 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (27.3 mg, 0.03912 mmol). MS (ESI) for C₃₄H₄₈ClN₆O₆S [M+H]⁺: m/z calcd, 703.29; found, 703.40. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₉H₄₀ClN₆O₄S [M+H]⁺: m/z calcd, 603.24; found, 603.34.

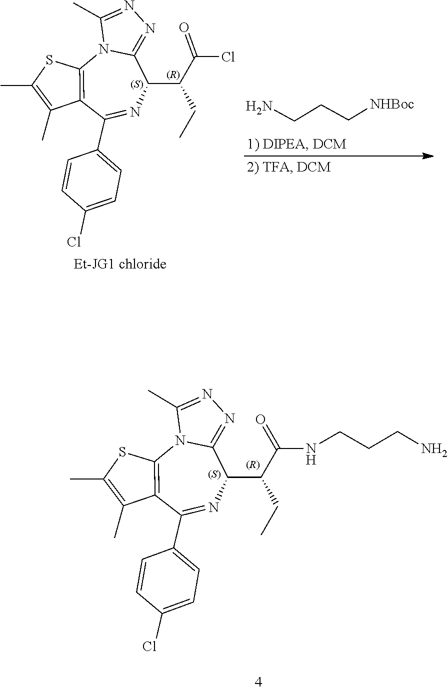

(R)-N-(3-aminopropyl)-2-((S)-4-(4-chlorophenyl)-2,3,9-trimethyl-6H-thieno[3,2-f][1,2,4]triazolo[4,3-a][1,4]diazepin-6-yl)butanamide (4). To a vial containing NHBoc-C3-amine (12.06 mg, 0.06922 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (22.7 mg, 0.03912 mmol). MS (ESI) for C₂₉H₃₈ClN₆O₃S [M+H]⁺: m/z calcd, 585.23; found, 585.34. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₄H₃₀ClN₆OS [M+H]⁺: m/z calcd, 485.18; found, 485.21.

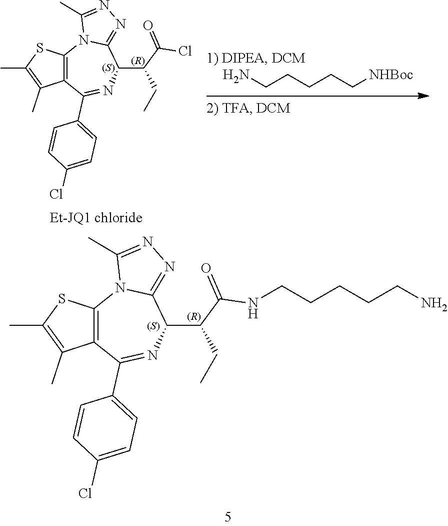

(R)-N-(5-aminopentyl)-2-((S)-4-(4-chlorophenyl)-2,3,9-trimethyl-6H-thieno[3,2-f][1,2,4]triazolo[4,3-a][1,4]diazepin-6-yl)butanamide (5). To a vial containing NHBoc-C5-amine (17.70 mg, 0.08749 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (23.8 mg, 0.03912 mmol). MS (ESI) for C₃₁H₄₂ClN₆O₃S [M+H]⁺: m/z calcd, 613.26; found, 613.34. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₆H₃₄ClN₆OS [M+H]⁺: m/z calcd, 513.21; found, 513.22.

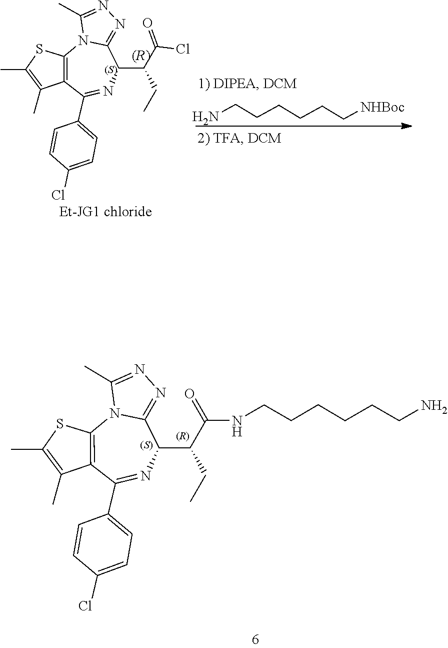

(R)-N-(6-aminohexyl)-2-((S)-4-(4-chlorophenyl)-2,3,9-trimethyl-6H-thieno[3,2-f][1,2,4]triazolo[4,3-a][1,4]diazepin-6-yl)butanamide (6). To a vial containing NHBoc-C6-amine (2.67 mg, 0.01230 mmol), and DIPEA (4.19 μL, 0.02410 mmol), was added a ˜2 mL dichloromethane aliquot of Et-JQ1 chloride (2.58 mg, 0.00601 mmol). The reaction was stirred for approximately 30 minutes and subsequently purified via flash column chromatography using dichloromethane and methanol, to quantitatively yield the Boc-protected amine analog (3.70 mg, 0.00601 mmol). MS (ESI) for C₃₂H₄₄ClN₆O₃S [M+H]⁺: m/z calcd, 627.29; found, 627.34. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₇H₃₆ClN₆OS [M+H]⁺: m/z calcd, 527.23; found, 527.17.

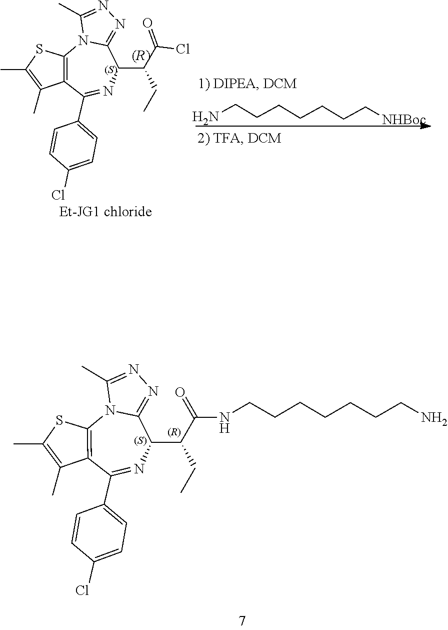

(R)-N-(7-aminoheptyl)-2-((S)-4-(4-chlorophenyl)-2,3,9-trimethyl-6H-thieno[3,2-f][1,2,4]triazolo[4,3-a][1,4]diazepin-6-yl)butanamide (7) To a vial containing NHBoc-C7-amine (38.16 mg, 0.1657 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (27.2 mg, 0.04245 mol). MS (ESI) for C₃₃H₄₆ClN₆O₃S [M+H]⁺: m/z calcd, 641.29; found, 641.40. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₈H₃₈ClN₆OS [M+H]⁺: m/z calcd, 541.24; found, 541.23.

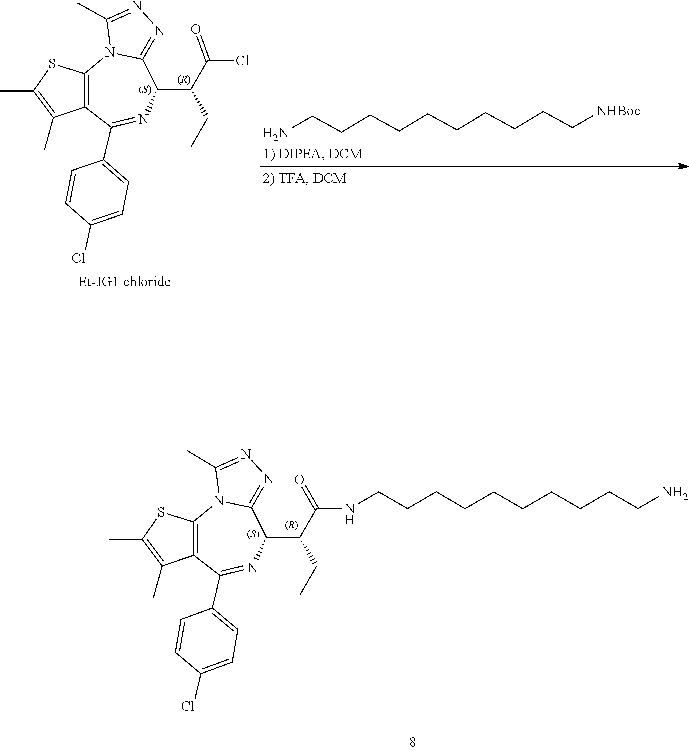

(R)-N-(10-aminodecyl)-2-((S)-4-(4-chlorophenyl)-2,3,9-trimethyl-6H-thieno[3,2-f][1,2,4]triazolo[4,3-a][1,4]diazepin-6-yl)butanamide (8). To a vial containing NHBoc-C10-amine (19.28 mg, 0.07077 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (26.5 mg, 0.03890 mmol). MS (ESI) for C₃₆H₅₂ClN₆O₃S [M+H]⁺: m/z calcd, 683.34; found, 683.45. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₃₁H₄₄ClN₆OS [M+H]⁺: m/z calcd, 583.30; found, 583.39.

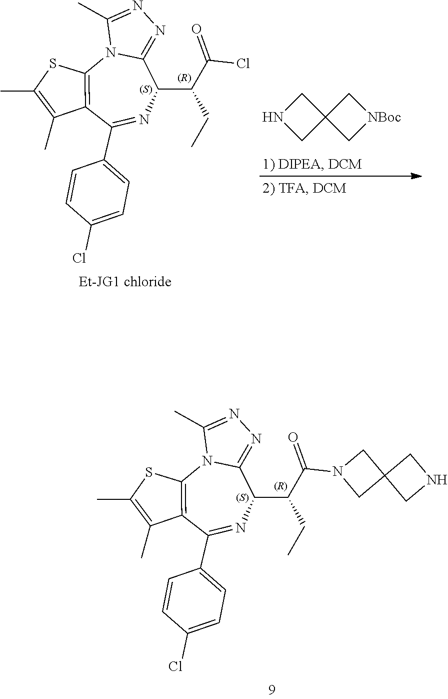

(R)-2-((S)-4-(4-chlorophenyl)-2,3,9-trimethyl-6H-thieno[3,2-f][1,2,4]triazolo[4,3-a][1,4]diazepin-6-yl)-1-(2,6-diazaspiro[3.3]heptan-2-yl)butan-1-one (9). To a vial containing tert-butyl 2,6-diazaspiro[3.3]heptane-6-carboxylate (15.56 mg, 0.07850 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (23.7 mg, 0.03890 mmol). MS (ESI) for C₃₁H₃₈ClN₆O₃S [M+H]⁺: m/z calcd, 609.23; found, 609.29. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₂₆H₃₀ClN₆OS [M+H]⁺: m/z calcd, 509.18; found, 509.17.

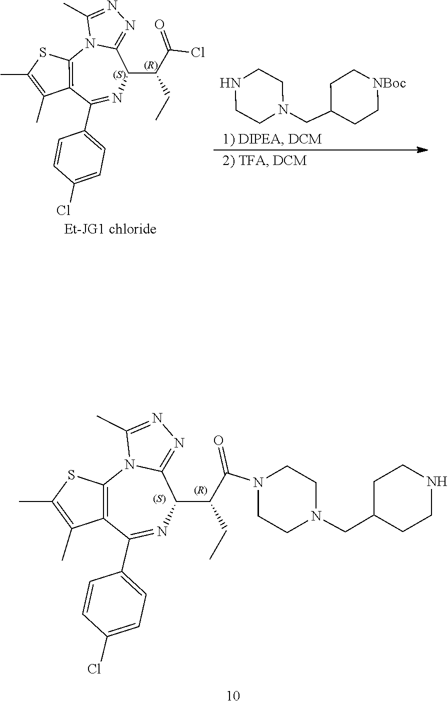

(R)-2-((S)-4-(4-chlorophenyl)-2,3,9-trimethyl-6H-thieno[3,2-f][1,2,4]triazolo[4,3-a][1,4]diazepin-6-yl)-1-(4-(piperidin-4-ylmethyl)piperazin-1-yl)butan-1-one (10). To a vial containing 1-Boc-4-piperazin-1-ylmethyl-piperidine (14.66 mg, 0.05172 mmol), and DIPEA (20.4 μL, 0.1174 mmol), was added a ˜2 mL dichloromethane aliquot of Et-JQ1 chloride (16.78 mg, 0.03912 mmol). The reaction was stirred for approximately 30 minutes and subsequently purified via flash column chromatography using dichloromethane and methanol, to quantitatively yield the Boc-protected amine analog (27.0 mg, 0.03890 mmol). MS (ESI) for C₃₆H₄₉ClN₇O₃S [M+H]⁺: m/z calcd, 694.32; found, 694.45. The product from the first step was redissolved and stirred in a solution of 2 mL dichloromethane and 0.5 mL trifluoroacetic acid. After ˜30 minutes, the reaction vial was dried under reduced pressure while azeotroping with dichloromethane to remove residual trifluoracetic acid. A quantitative yield was assumed, and the product was carried on to the next step. MS (ESI) for C₃₁H₄₁ClN₇OS [M+H]⁺: m/z calcd, 594.27; found, 594.34.

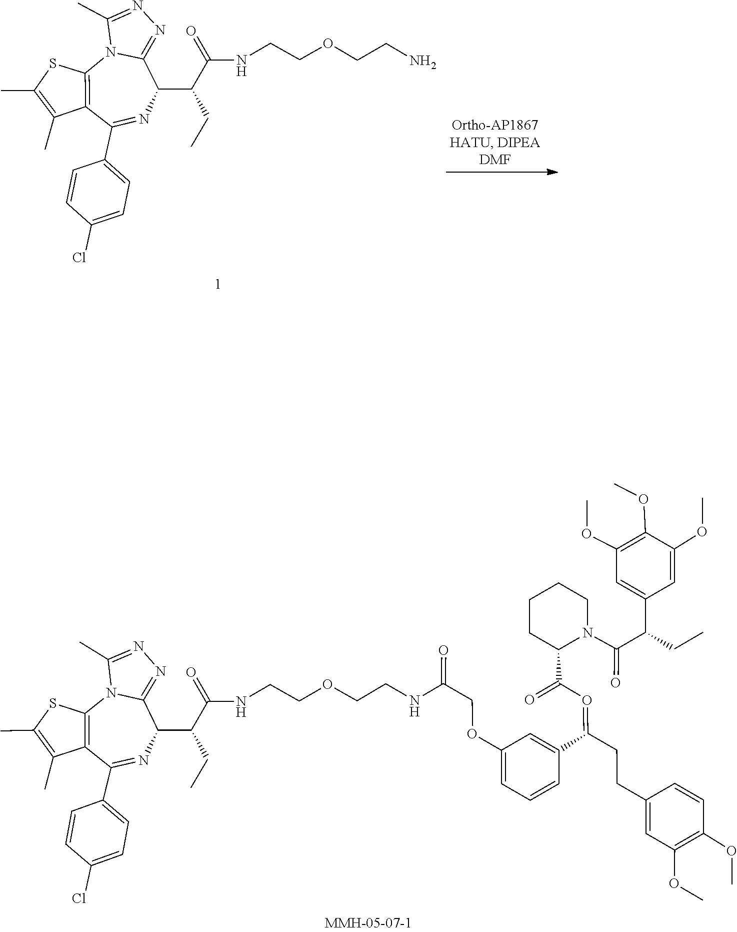

(R)-1-(3-(2-((2-(2-((R)-2-((S)-4-(4-chlorophenyl)-2,3,9-trimethyl-6H-thieno[3,2-f][1,2,4]triazolo[4,3-a][1,4]diazepin-6-yl)butanamido)ethoxy)ethyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-1). DIPEA (17.5 μL, 0.1014 mmol), ortho-AP1867 (9.95 mg, 0.01434 mmol), and HATU (7.44 mg, 0.01956 mmol) were added to an aliquot of 1 (6.72 mg, 0.01304 mmol) in 2 mL DMF. DIPEA (10 μL, 0.05796 mmol) was further added to ensure the completion of reaction progress, and the crude mixture was directly purified using HPLC (method 1, no TFA additive) to yield MMH-05-07-1 (8.92 mg, 0.00749 mmol, 57.4% yield). MS (ESI) for C₆₃H₇₈ClN₇O₁₂S [M+H]⁺: m/z calcd, 1190.50; found, 1190.36.

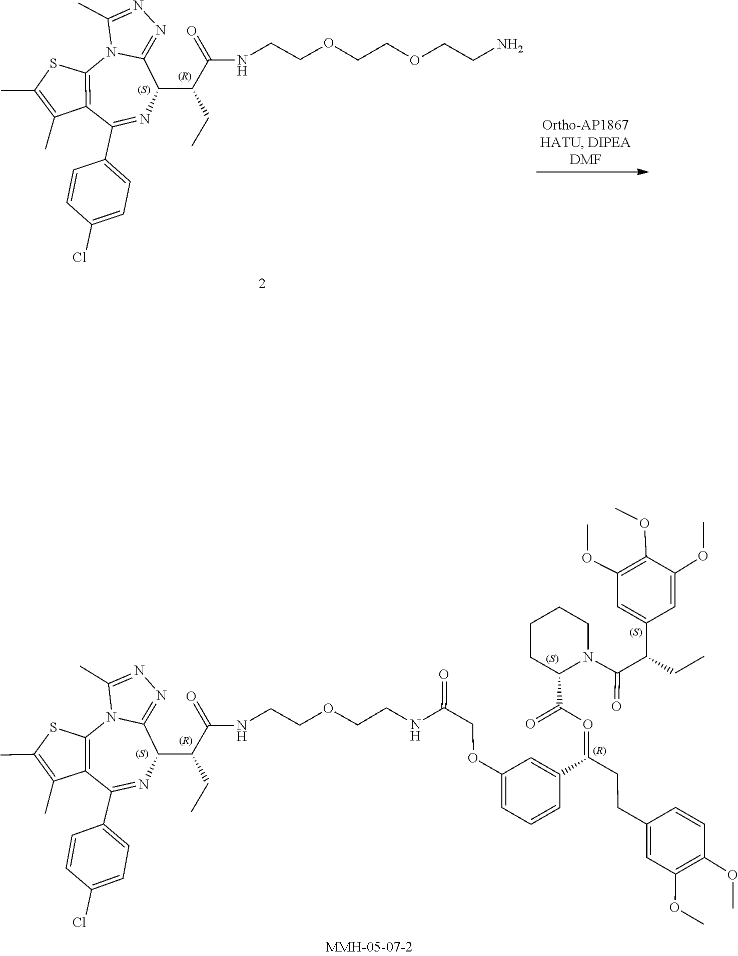

(R)-1-(3-(((R)-14-((S)-4-(4-chlorophenyl)-2,3,9-trimethyl-6H-thieno[3,2-f][1,2,4]triazolo[4,3-a][1,4]diazepin-6-yl)-2,13-dioxo-6,9-dioxa-3,12-diazahexadecyl)oxy)phenyl)-3-(3,4-dimethoxyphenyl)propyl (S)-1-((S)-2-(3,4,5-trimethoxyphenyl)butanoyl)piperidine-2-carboxylate (MMH-05-07-2). DIPEA (17.5 μL, 0.1014 mmol), ortho-AP1867 (9.95 mg, 0.01434 mmol), and HATU (7.44 mg, 0.01956 mmol) were added to an aliquot of 2 (7.29 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-2 (6.73 mg, 0.00545 mmol, 41.8% yield). MS (ESI) for C₆₅H₈₁ClN₇O₁₃S [M+H]⁺: m/z calcd, 1234.52; found, 1234.81.

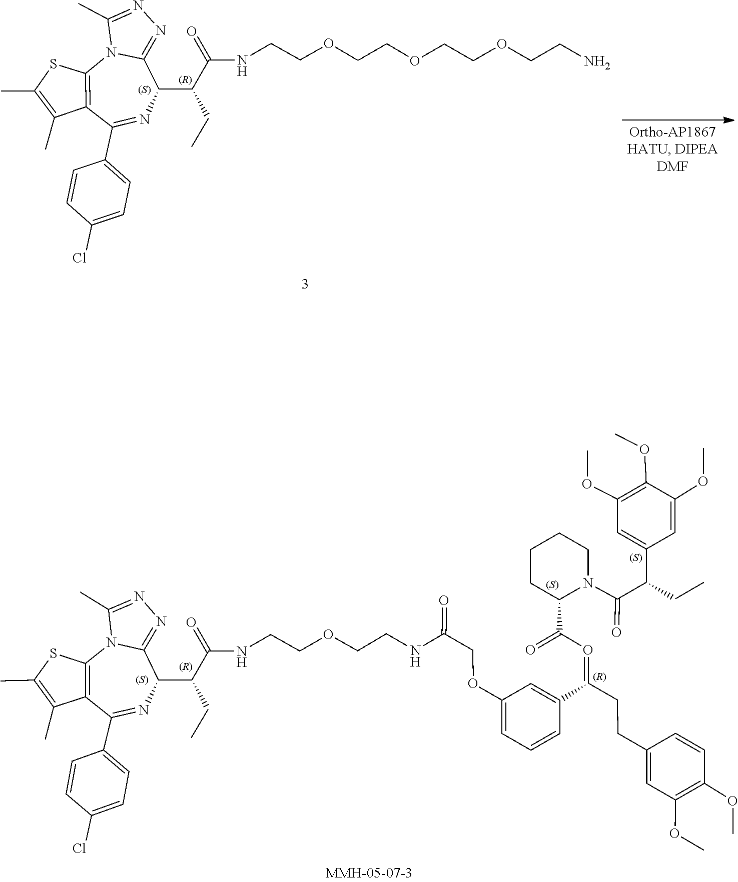

(R)-1-(3-(((R)-17-((S)-4-(4-chlorophenyl)-2,3,9-trimethyl-6H-thieno[3,2-f][1,2,4]triazolo[4,3-a][1,4]diazepin-6-yl)-2,16-dioxo-6,9,12-trioxa-3,15-diazanonadecyl)oxy)phenyl)-3-(3,4-dimethoxyphenyl)propyl (S)-1-((S)-2-(3,4,5-trimethoxyphenyl)butanoyl)piperidine-2-carboxylate (MMH-05-07-3). DIPEA (17.5 μL, 0.1014 mmol), ortho-AP1867 (9.95 mg, 0.01434 mmol), and HATU (7.44 mg, 0.01956 mmol) were added to an aliquot of 3 (7.87 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-3 (10.3 mg, 0.00802 mmol, 61.5% yield). MS (ESI) for fragment C₄₈H₅₈ClN₆O₈S [M−H]⁺: m/z calcd, 913.38; found, 913.31. MS (ESI) for fragment C₁₉H₂₈NO₆ [M+H]⁺: m/z calcd, 366.18; found, 366.17.

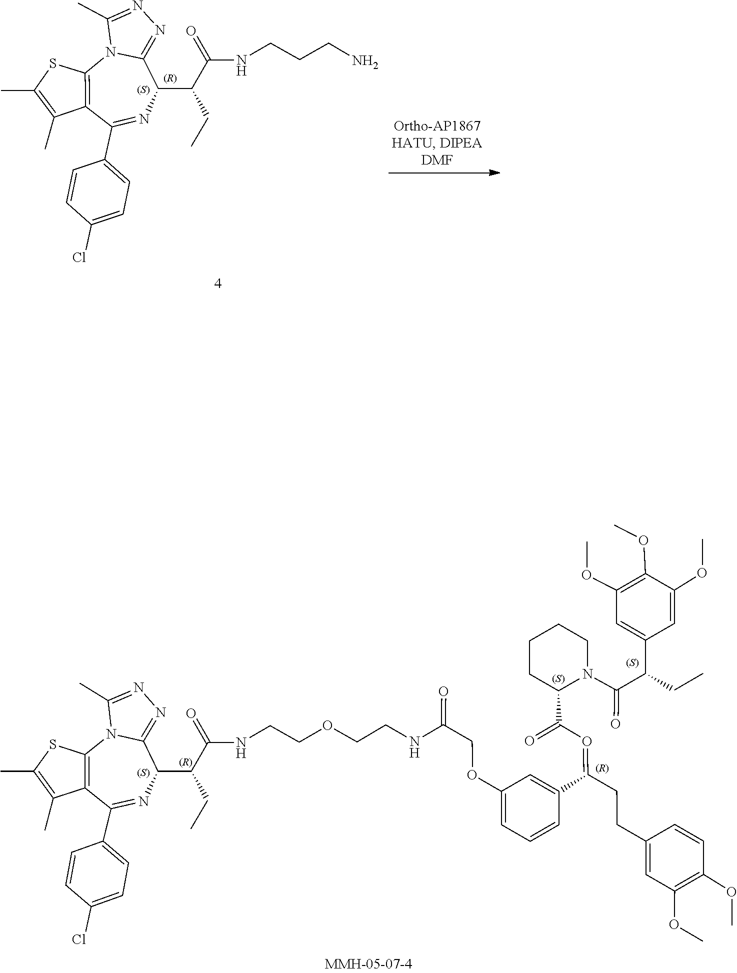

(R)-1-(3-(2-((3-((R)-2-((S)-4-(4-chlorophenyl)-2,3,9-trimethyl-6H-thieno[3,2-f][1,2,4]triazolo[4,3-a][1,4]diazepin-6-yl)butanamido)propyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-4). DIPEA (17.5 μL, 0.1014 mmol), ortho-AP1867 (9.95 mg, 0.01434 mmol), and HATU (7.44 mg, 0.01956 mmol) were added to an aliquot of 4 (6.33 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-4 (8.58 mg, 0.00739 mmol, 56.7% yield). MS (ESI) for C₆₂H₇₅ClN₇O₁₁S [M+H]⁺: m/z calcd, 1160.49; found, 1160.43.

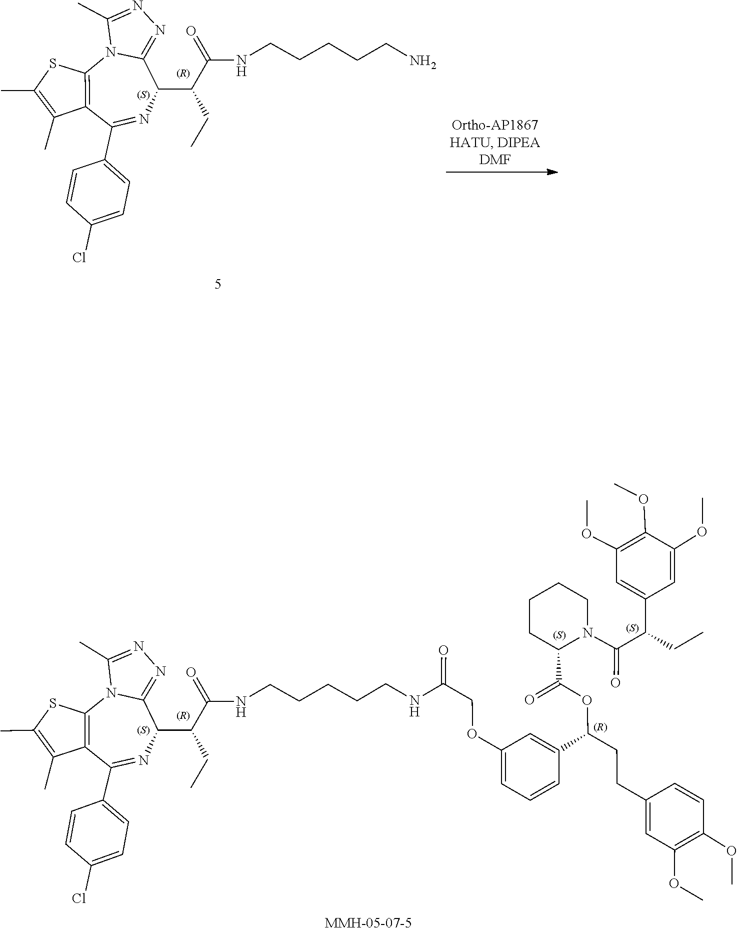

(R)-1-(3-(2-((5-((R)-2-((S)-4-(4-chlorophenyl)-2,3,9-trimethyl-6H-thieno[3,2-f][1,2,4]triazolo[4,3-a][1,4]diazepin-6-yl)butanamido)pentyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-5). DIPEA (17.5 μL, 0.1014 mmol), ortho-AP1867 (9.95 mg, 0.01434 mmol), and HATU (7.44 mg, 0.01956 mmol) were added to an aliquot of 5 (6.69 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-5 (8.97 mg, 0.00754 mmol, 57.9% yield). MS (ESI) for C₆₄H₇₉ClN₇O₁₁S [M+H]⁺: m/z calcd, 1188.52; found, 1188.31.

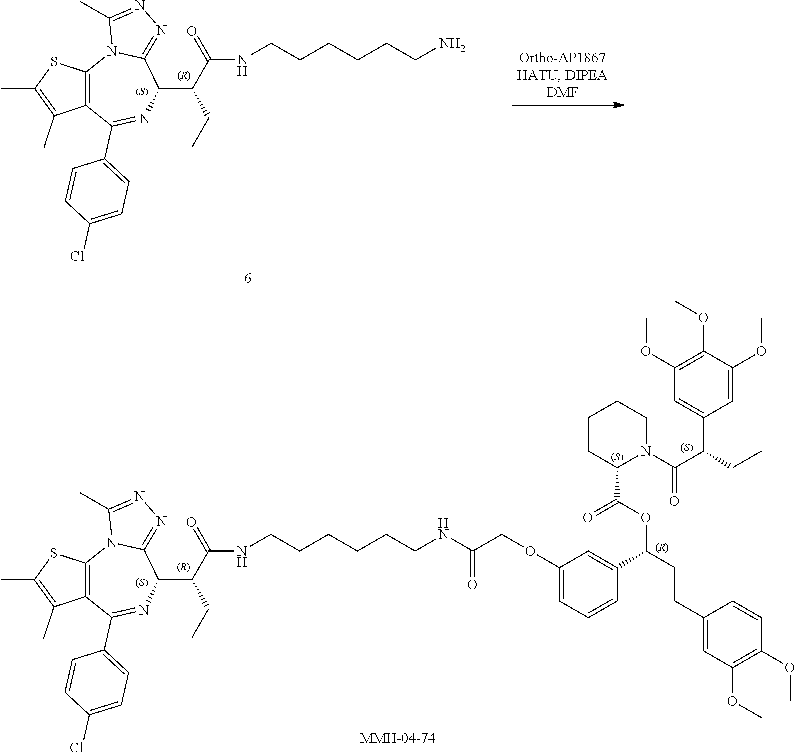

(R)-1-(3-(2-((6-((R)-2-((S)-4-(4-chlorophenyl)-2,3,9-trimethyl-6H-thieno[3,2-f][1,2,4]triazolo[4,3-a][1,4]diazepin-6-yl)butanamido)hexyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-04-74). To a solution of 6 (3.17 mg, 0.00601 mmol) in 1 mL dichloromethane, was added a 1 mL dichloromethane solution of DIPEA (3.14 μL, 0.0180 mmol) and ortho-AP1867 (4.17 mg, 0.00601 mmol). HATU (4.12 mg, 0.0108 mmol) was immediately added and the reaction was left to stir for approximately 10 minutes. The crude solution was washed three times with a saturated aqueous solution of ammonium chloride, twice with milliQ water, and once with brine. The organic layer was dried with sodium sulfate and purified via a combi flash (EtOAc and Hex) followed by flushing by HPLC (no TFA; 5-minute flush at 50:50 MeOH and milliQ water followed by a methanol flush for 10 minutes): to yield MMH-04-74 (5.76 mg, 0.00601 mmol, 79.6% yield). MS (ESI) for C₆₅H₈₁ClN₇O₁₁S [M+H]⁺: m/z calcd, 1202.53; found, 1202.52.

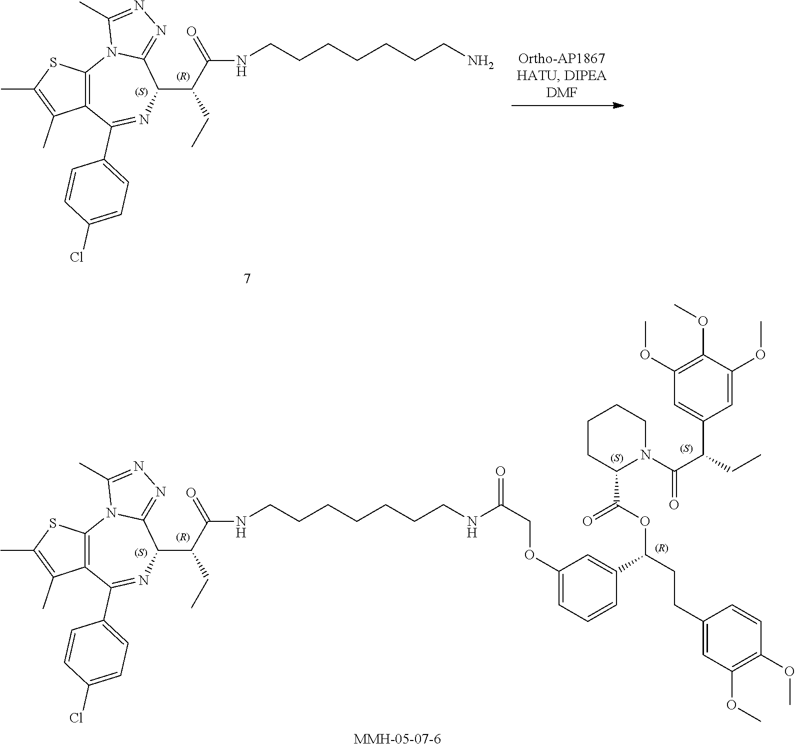

(R)-1-(3-(2-((7-((R)-2-((S)-4-(4-chlorophenyl)-2,3,9-trimethyl-6H-thieno[3,2-f][1,2,4]triazolo[4,3-a][1,4]diazepin-6-yl)butanamido)heptyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-6). DIPEA (17.5 μL, 0.1014 mmol), ortho-AP1867 (9.95 mg, 0.01434 mmol), and HATU (7.44 mg, 0.01956 mmol) were added to an aliquot of 7 (7.06 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-6 (8.79 mg, 0.00722 mmol, 55.4% yield). MS (ESI) for C₆₆H₈₃ClN₇O₁₁S [M+H]⁺: m/z calcd, 1216.55; found, 1216.29.

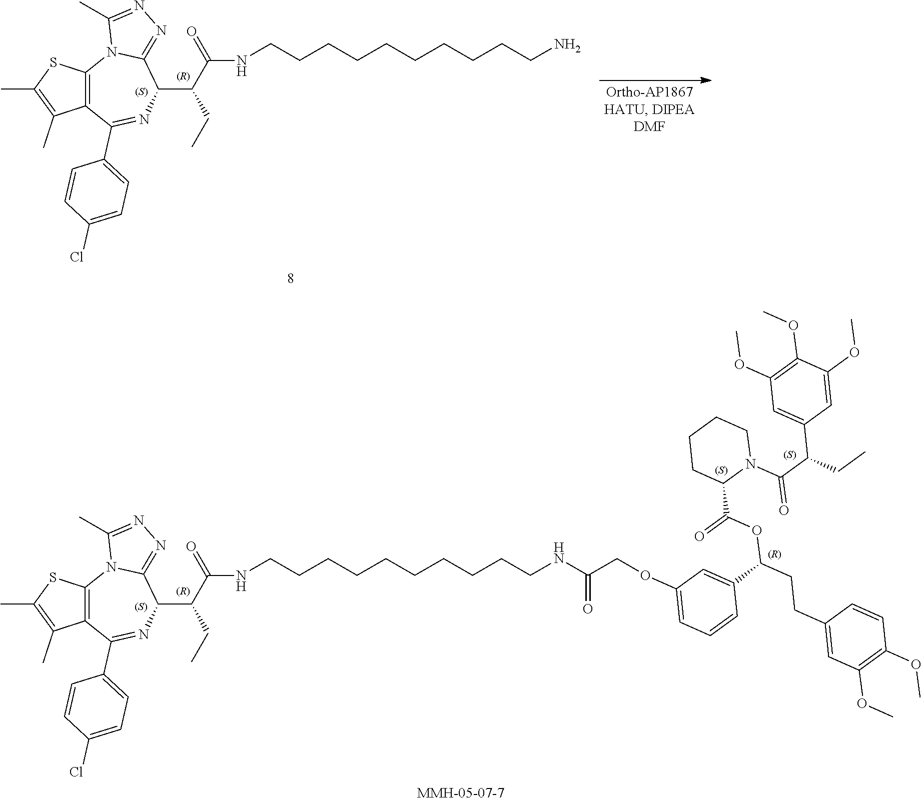

(R)-1-(3-(2-((10-((R)-2-((S)-4-(4-chlorophenyl)-2,3,9-trimethyl-6H-thieno[3,2-f][1,2,4]triazolo[4,3-a][1,4]diazepin-6-yl)butanamido)decyl)amino)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-7). DIPEA (17.5 μL, 0.1014 mmol), ortho-AP1867 (9.95 mg, 0.01434 mmol), and HATU (7.44 mg, 0.01956 mmol) were added to an aliquot of 8 (7.61 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-7 (6.45 mg, 0.00512 mmol, 39.3% yield). MS (ESI) for fragment C₅₀H₆₂ClN₆O₅S [M−H]⁺: m/z calcd, 893.43; found, 893.31. MS (ESI) for fragment C₁₉H₂₈NO₆ [M+H]⁺: m/z calcd, 366.18; found, 366.17.

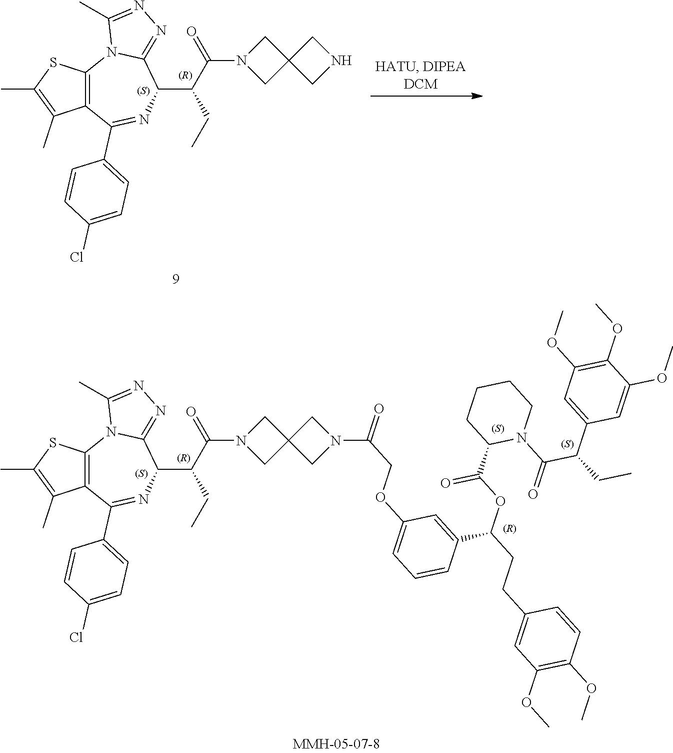

(R)-1-(3-(2-(6-((R)-2-((S)-4-(4-chlorophenyl)-2,3,9-trimethyl-6H-thieno[3,2-f][1,2,4]triazolo[4,3-a][1,4]diazepin-6-yl)butanoyl)-2,6-diazaspiro[3.3]heptan-2-yl)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-8). DIPEA (17.5 μL, 0.1014 mmol), ortho-AP1867 (9.95 mg, 0.01434 mmol), and HATU (7.44 mg, 0.01956 mmol) were added to an aliquot of 9 (6.64 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-8 (8.64 mg, 0.00729 mmol, 55.9% yield). MS (ESI) for C₆₄H₇₅ClN₇O₁₁S [M+H]⁺: m/z calcd, 1184.49; found, 1184.35.

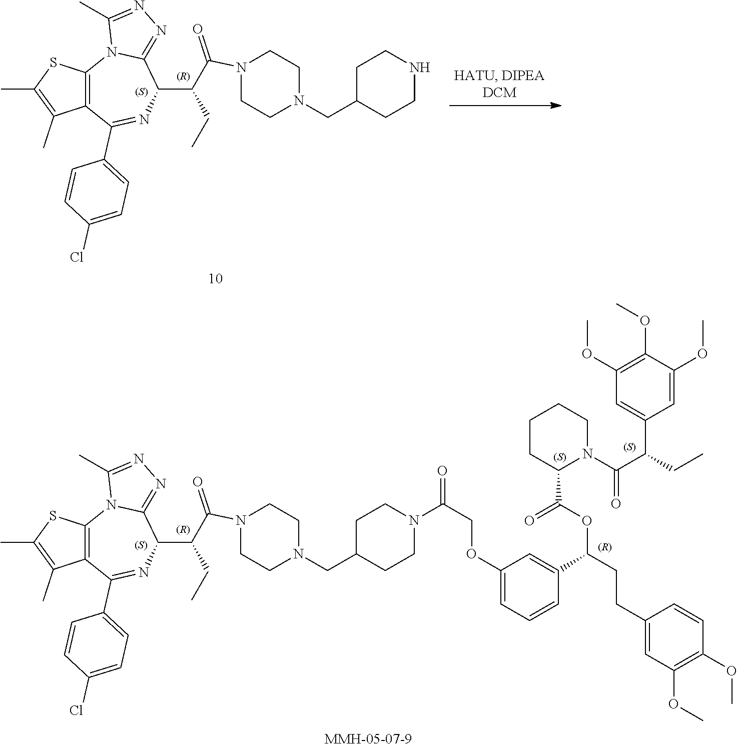

(R)-1-(3-(2-(4-((4-((R)-2-((S)-4-(4-chlorophenyl)-2,3,9-trimethyl-6H-thieno[3,2-f][1,2,4]triazolo[4,3-a][1,4]diazepin-6-yl)butanoyl)piperazin-1-yl)methyl)piperidin-1-yl)-2-oxoethoxy)phenyl)-3-(3,4-dimethoxyphenyl)propyl (S)-1-((S)-2-(3,4,5-trimethoxyphenyl)butanoyl)piperidine-2-carboxylate (MMH-05-07-9). DIPEA (17.5 μL, 0.1014 mmol), ortho-AP1867 (9.95 mg, 0.01434 mmol), and HATU (7.44 mg, 0.01956 mmol) were added to an aliquot of 10 (7.75 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-07-9 (2.66 mg, 0.00209 mmol, 16.1% yield). MS (ESI) for fragment C₅₀H₅₉ClN₇O₅S [M−H]⁺: m/z calcd, 904.41; found, 904.36. MS (ESI) for fragment C₁₉H₂₈NO₆ [M+H]⁺: m/z calcd, 366.18; found, 366.17.

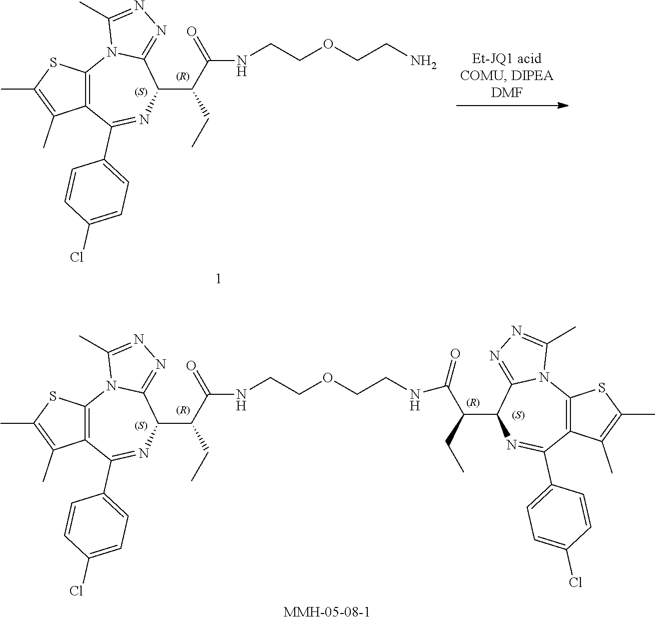

(2R,2′R)-N,N′-(oxybis(ethane-2,1-diyl))bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-1). DIPEA (17.5 μL, 0.1014 mmol), Et-JQ1 acid (6.15 mg, 0.01434 mmol), and COMU (8.38 mg, 0.01956 mmol) were added to an aliquot of 1 (6.72 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-08-1 (4.80 mg, 0.00520 mmol, 40.0% yield). MS (ESI) for C₄₆H₅₁Cl₂N₁₀O₃S₂ [M+H]⁺: m/z calcd, 925.29; found, 925.26.

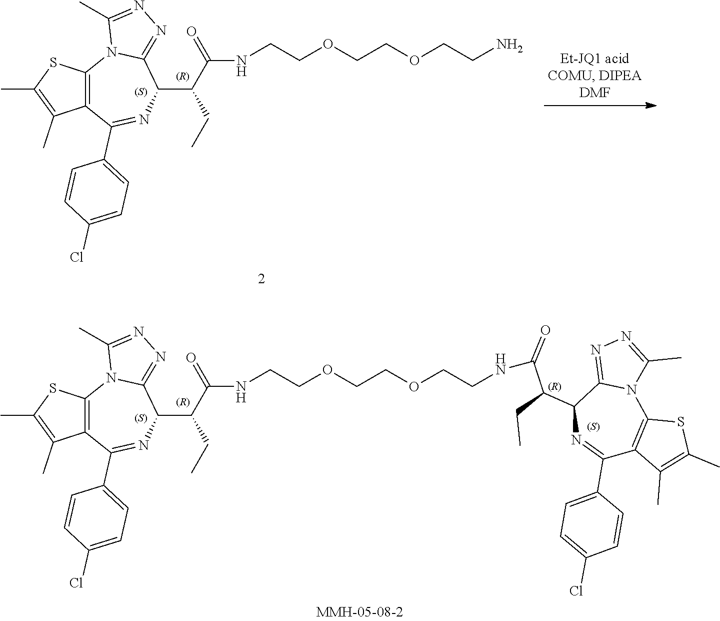

(2R,2′R)-N,N′-((ethane-1,2-diylbis(oxy))bis(ethane-2,1-diyl))bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-2). DIPEA (17.5 μL, 0.1014 mmol), Et-JQ1 acid (6.15 mg, 0.01434 mmol), and COMU (8.38 mg, 0.01956 mmol) were added to an aliquot of 2 (7.29 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-08-2 (2.24 mg, 0.00231 mmol, 17.7% yield). MS (ESI) for C₄₈H₅₅Cl₂N₁₀O₄S₂ [M+H]⁺: m/z calcd, 969.31; found, 969.31.

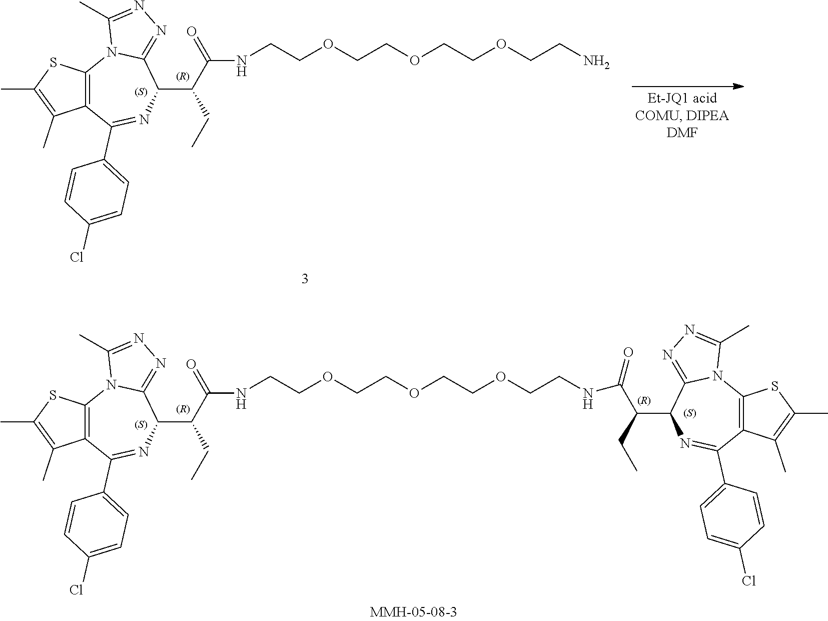

(2R,2′R)-N,N′-(((oxybis(ethane-2,1-diyl))bis(oxy))bis(ethane-2,1-diyl))bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-3). DIPEA (17.5 μL, 0.1014 mmol), Et-JQ1 acid (6.15 mg, 0.01434 mmol), and COMU (8.38 mg, 0.01956 mmol) were added to an aliquot of 3 (7.87 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-08-3 (6.72 mg, 0.00663 mmol, 50.8% yield). MS (ESI) for C₅₀H₆₀Cl₂N₁₀O₅S₂ [M+2H]²⁺: m/z calcd, 507.17; found, 507.27.

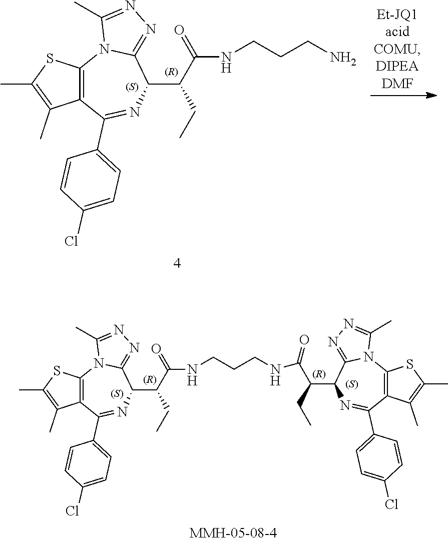

(2R,2′R)-N,N′-(propane-1,3-diyl)bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-4). DIPEA (17.5 μL, 0.1014 mmol), Et-JQ1 acid (6.15 mg, 0.01434 mmol), and COMU (8.38 mg, 0.01956 mmol) were added to an aliquot of 4 (6.33 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-08-4 (4.67 mg, 0.00521 mmol, 40.0% yield). MS (ESI) for C₄₅H₄₉Cl₂N₁₀O₂S₂ [M+H]⁺: m/z calcd, 895.29; found, 895.31.

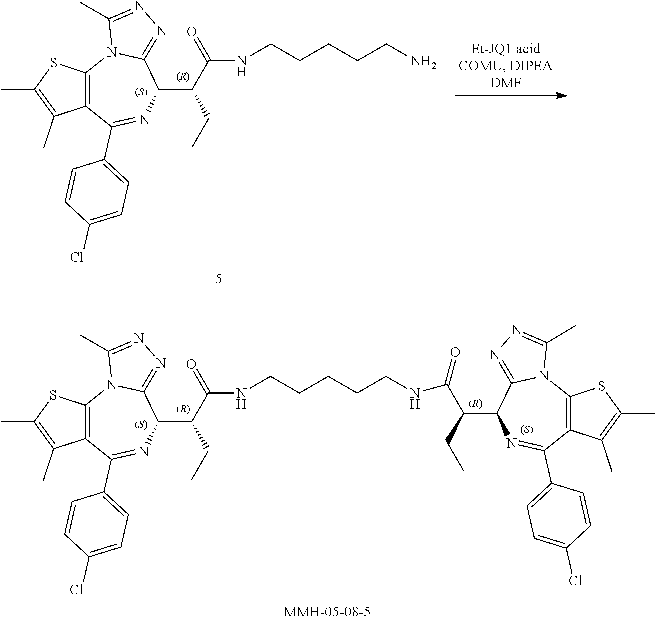

(2R,2′R)-N,N′-(pentane-1,5-diyl)bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-5). DIPEA (17.5 μL, 0.1014 mmol), Et-JQ1 acid (6.15 mg, 0.01434 mmol), and COMU (8.38 mg, 0.01956 mmol) were added to an aliquot of 5 (6.69 mg, 0.01304 mmol) in 2 mL DMF. DIPEA (10.0 μL, 0.05839 mmol) was further added to ensure the completion of reaction progress, and the crude mixture was directly purified using HPLC (method 1, no TFA additive) to yield MMH-05-08-5 (5.40 mg, 0.00580 mmol, 45.0% yield). MS (ESI) for C₄₇H₅₃Cl₂N₁₀O₂S₂ [M+H]⁺: m/z calcd, 923.31; found, 923.26.

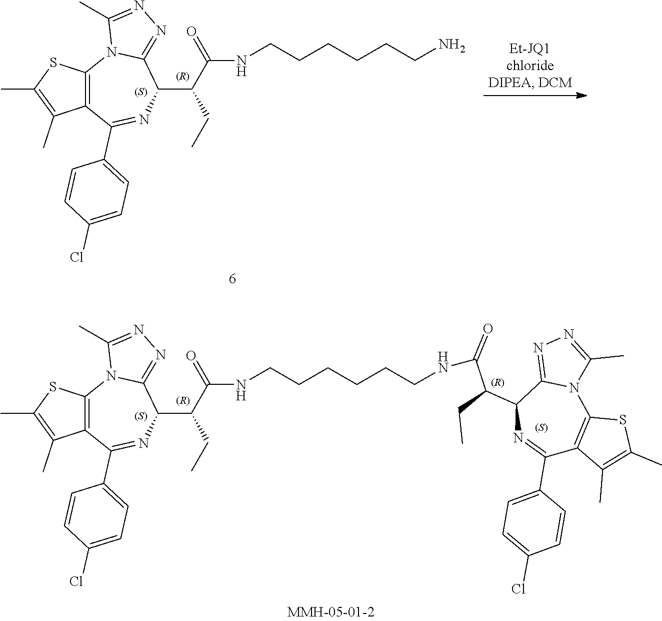

(2R,2′R)-N,N′-(hexane-1,6-diyl)bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-01-2). To a vial containing Et-JQ1 chloride (1.12 mg, 0.00251 mmol) in 2 mL dichloromethane, was added DIPEA (10.5 μL, 0.06020 mmol), and 6 (1.32 mg, 0.00251 mmol). The reaction mixture was then purified via combiflash using a gradient of methanol in dichlormethane to yield MMH-05-01-2 (1.47 mg, 0.00157 mmol, 62.5% yield). MS (ESI) for C₄₈H₅₅Cl₂N₁₀O₂S₂ [M+H]⁺: m/z calcd, 937.33; found, 937.36.

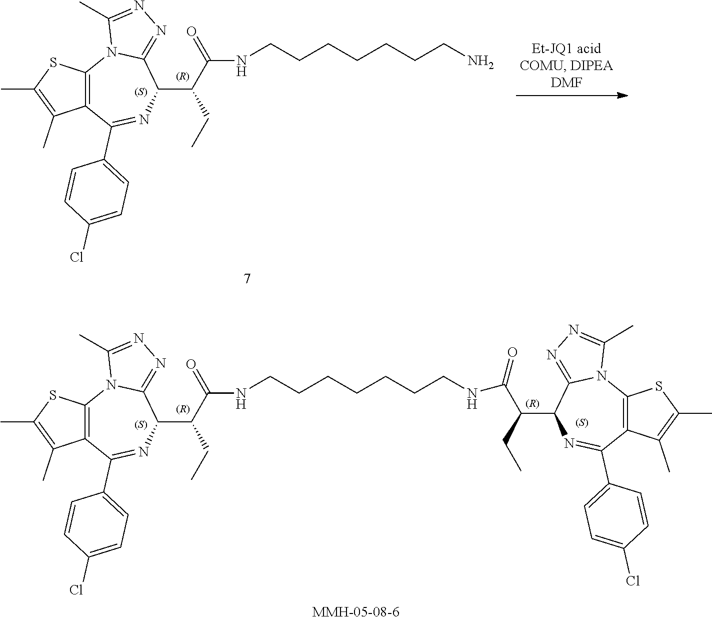

(2R,2′R)-N,N′-(heptane-1,7-diyl)bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-6). DIPEA (17.5 μL, 0.1014 mmol), Et-JQ1 acid (6.15 mg, 0.01434 mmol), and COMU (8.38 mg, 0.01956 mmol) were added to an aliquot of 7 (7.06 mg, 0.01304 mmol) in 2 mL DMF. DIPEA (20.0 μL, 0.1168 mmol) was further added to ensure the completion of reaction progress, and the crude mixture was directly purified using HPLC (method 1, no TFA additive) to yield MMH-05-08-6 (1.37 mg, 0.00144 mmol, 11.0% yield). MS (ESI) for C₄₉H₅₇Cl₂N₁₀O₂S₂ [M+H]⁺: m/z calcd, 951.34; found, 951.41.

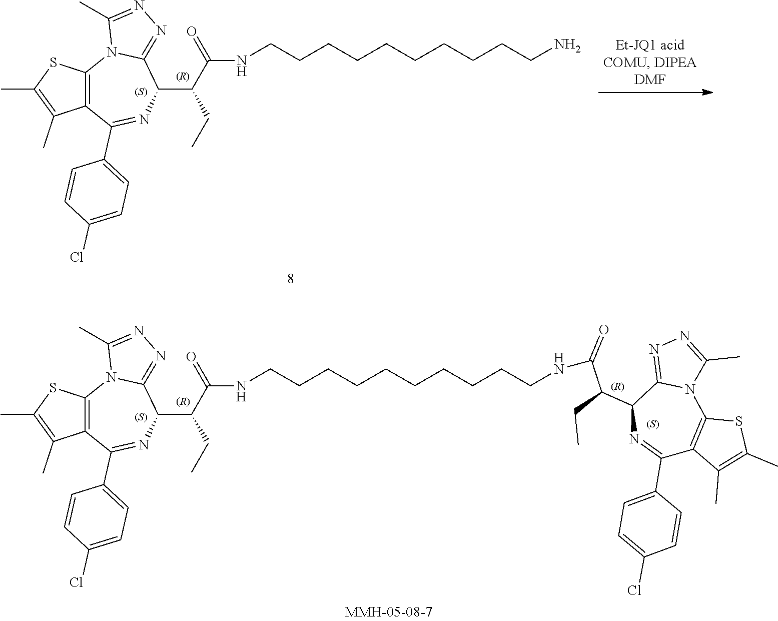

(2R,2′R)-N,N′-(decane-1,10-diyl)bis(2-((S)-4-(4-chlorophenyl)-2,3,9-trimethyl-6H-thieno[3,2-f][1,2,4]triazolo[4,3-a][1,4]diazepin-6-yl)butanamide) (MMH-05-08-7). DIPEA (17.5 μL, 0.1014 mmol), Et-JQ1 acid (6.15 mg, 0.01434 mmol), and COMU (8.38 mg, 0.01956 mmol) were added to an aliquot of 8 (7.61 mg, 0.01304 mmol) in 2 mL DMF. DIPEA (20.0 μL, 0.1168 mmol) was further added to ensure the completion of reaction progress, and the crude mixture was directly purified using HPLC (method 1, no TFA additive) to yield MMH-05-08-7 (2.02 mg, 0.00203 mmol, 15.6% yield). MS (ESI) for C₅₂H₆₄Cl₂N₁₀O₂S₂ [M+2H]²⁺: m/z calcd, 497.19; found, 497.32.

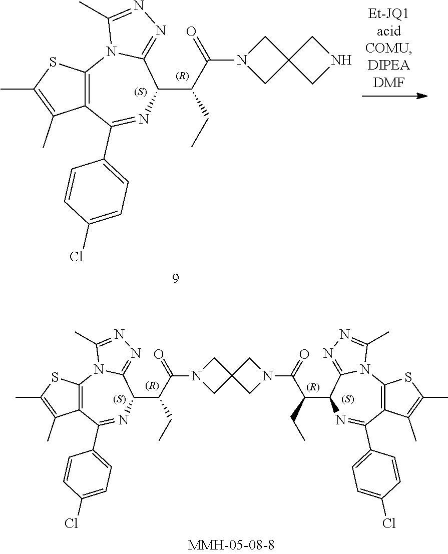

(2R,2′R)-1,1′-(2,6-diazaspiro[3.3]heptane-2,6-diyl)bis(2-((S)-4-(4-chlorophenyl)-2,3,9-trimethyl-6H-thieno[3,2-f][1,2,4]triazolo[4,3-a][1,4]diazepin-6-yl)butan-1-one) (MMH-05-08-8). DIPEA (17.5 μL, 0.1014 mmol), Et-JQ1 acid (6.15 mg, 0.01434 mmol), and COMU (8.38 mg, 0.01956 mmol) were added to an aliquot of 9 (6.64 mg, 0.01304 mmol) in 2 mL DMF. DIPEA (120.0 μL, 0.7008 mmol) was further added to ensure the completion of reaction progress, and the crude mixture was directly purified using HPLC (method 1, no TFA additive) to yield MMH-05-08-8 (1.48 mg, 0.00161 mmol, 12.63 yield). MS (ESI) for C₄₇H₄₉Cl₂N₁₀O₂S₂ [M+H]⁺: m/z calcd, 919.28; found, 919.31.

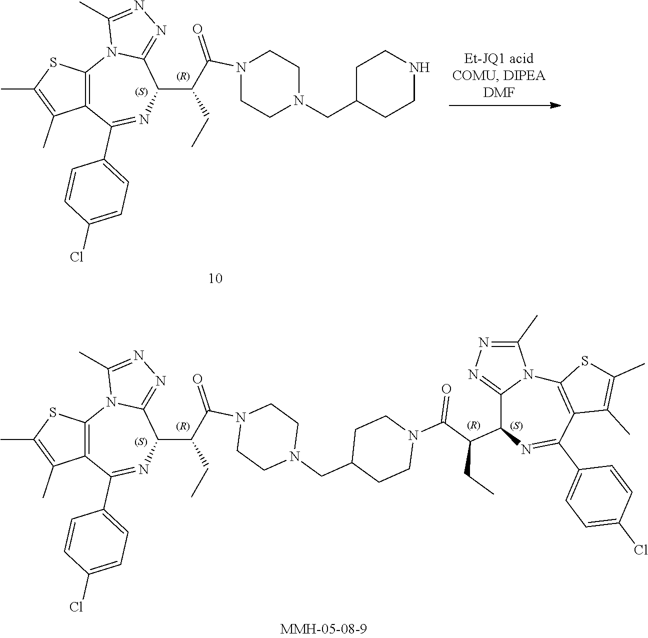

(R)-2-((S)-4-(4-chlorophenyl)-2,3,9-trimethyl-6H-thieno[3,2-f][1,2,4]triazolo[4,3-a][1,4]diazepin-6-yl)-1-(4-((4-((R)-2-((S)-4-(4-chlorophenyl)-2,3,9-trimethyl-6H-thieno[3,2-f][1,2,4]triazolo[4,3-a][1,4]diazepin-6-yl)butanoyl)piperazin-1-yl)methyl)piperidin-1-yl)butan-1-one (MMH-05-08-9). DIPEA (17.5 μL, 0.1014 mmol), Et-JQ1 acid (6.15 mg, 0.01434 mmol), and COMU (8.38 mg, 0.01956 mmol) were added to an aliquot of 10 (7.75 mg, 0.01304 mmol) in 2 mL DMF. DIPEA (120.0 μL, 0.7008 mmol) was further added to ensure the completion of reaction progress, and the crude mixture was directly purified using HPLC (method 1, no TFA additive) to yield MMH-05-08-9 (5.70 mg, 0.00570 mmol, 43.0% yield). MS (ESI) for C₅₂H₆₀Cl₂N₁₁O₂S₂ [M+H]⁺: m/z calcd, 1004.37; found, 1004.37.

### Example 2

### Assays

FKBP/BromoTag NanoBiT-tagged construct cloning. FKBP12_{F36V} and BromoTag sequences were cloned using Gibson assembly into pBiT1.1-C [TK/LgBiT], pBiT2.1-C [TK/SmBiT], pBiT1.1-N [TK/LgBiT], and pBiT2.1-N [TK/SmBiT] vectors included in the NanoBiT® PPI MCS Starter System kit (Promega #N2014). Vectors were digested with EcoRI and NheI, inserts were amplified with PCR, and constructs were ligated using NEBuilder® HiFi DNA Assembly Master Mix. pBiT1.1 and pBiT2.1 vectors were linearized at EcoRI and NheI cut sites, inserts were amplified with PCR, and constructs were ligated using NEBuilder® HiFi DNA Assembly Master Mix.

To generate Caspase 9 and c-KIT intracellular domain (ICD) mammalian expression constructs, N-terminal FKBP12_{F36V}, BromoTag_{L387A}, 2x-BromoTag_{L387A}, FRB_{T1098L}, and FKBP_{WT} were cloned using Gibson assembly into pHR-Caspase 9 and pLX307-c-KIT ICD vectors. Self-cleaving co-expression constructs (pLX307 FKBP12_{F36V}-c-KIT ICD-[P2A+T2A]-BromoTag_{L387A}-c-KIT ICD, pLX307 FKBP12_{F36V}-c-KIT ICD-[P2A+T2A]-2x-BromoTag_{L387A}-c-KIT ICD, and pLX307 FRB_{T1098L}-c-KIT ICD-[P2A+T2A]-FKBP_{WT}-c-KIT ICD) were also generated using Gibson assembly. Vectors and inserts were amplified with PCR and ligated using NEBuilder® HiFi DNA Assembly Master Mix.

Amino acid linker sequences between dimerization tags and proteins of interest were as follows: Caspase 9, TRRPPPR; c-kit ICD, SGGS or GGGGSSGGS. P2A+T2A self-cleavage sequence was as follows: ATNFSLLKQAGDVEENPGP-GSG-EGRGSLLTCGDVEENPGP. 2x-BromoTag_{L387A} constructs contained both a wild-type and a codon optimized BromoTag_{L387A} sequence separated by a flexible SGGS or rigid TR linker.

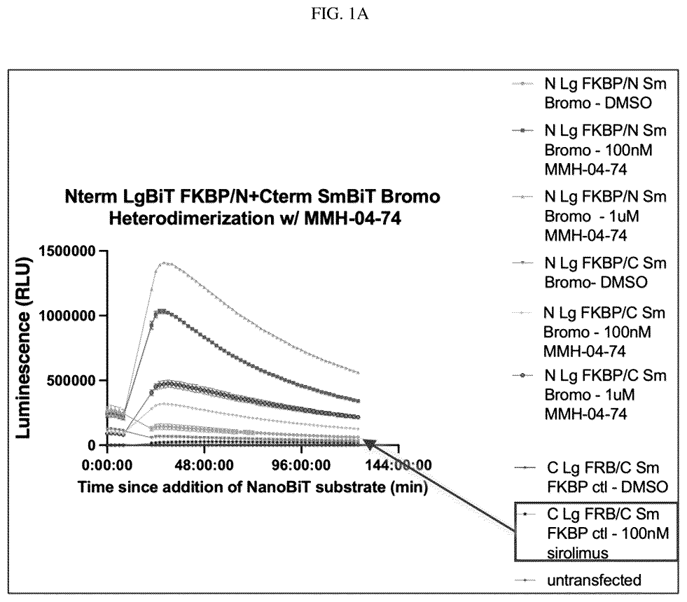

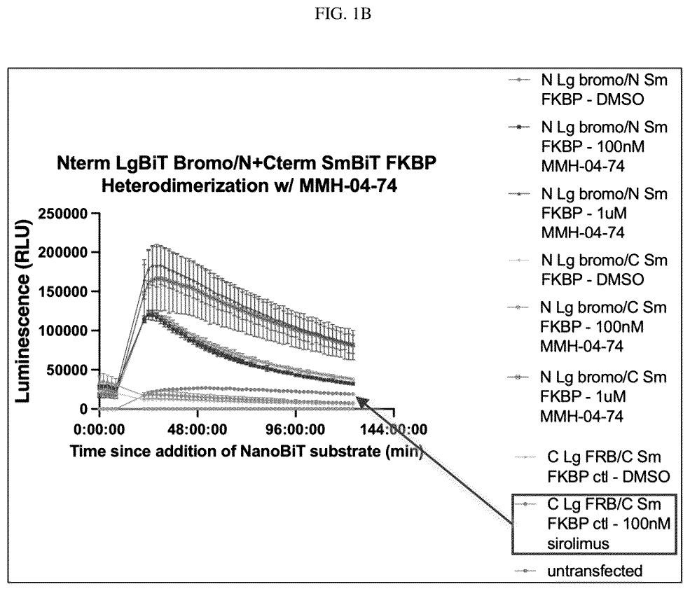

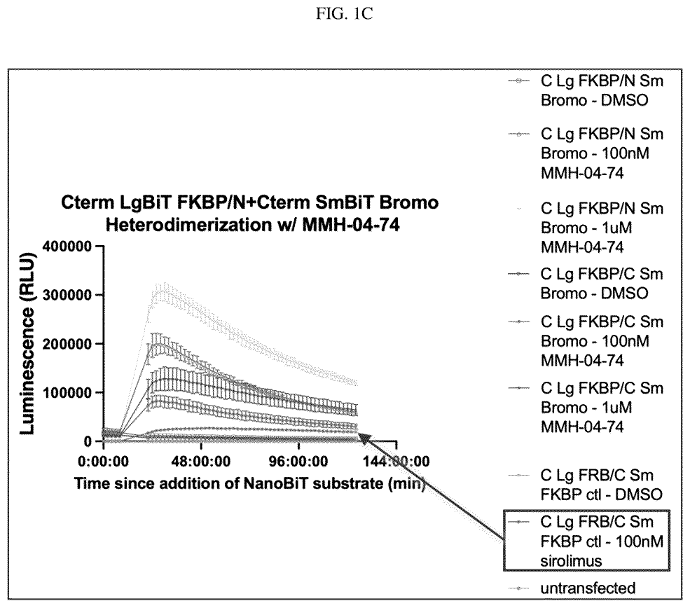

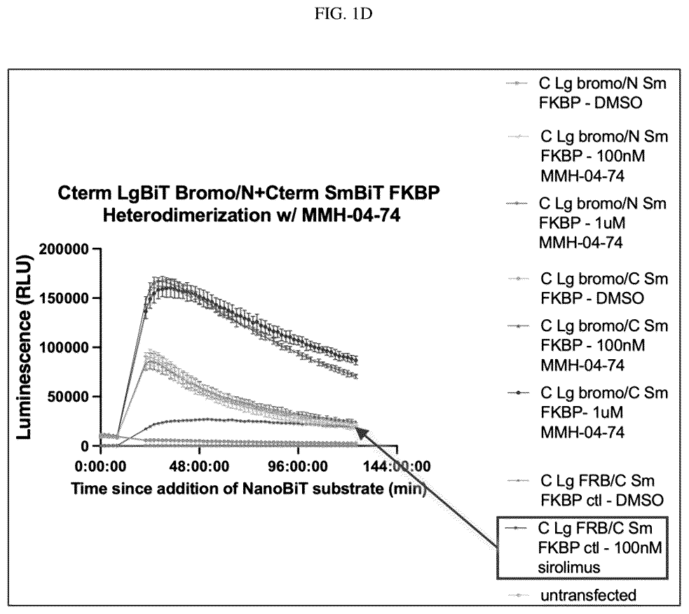

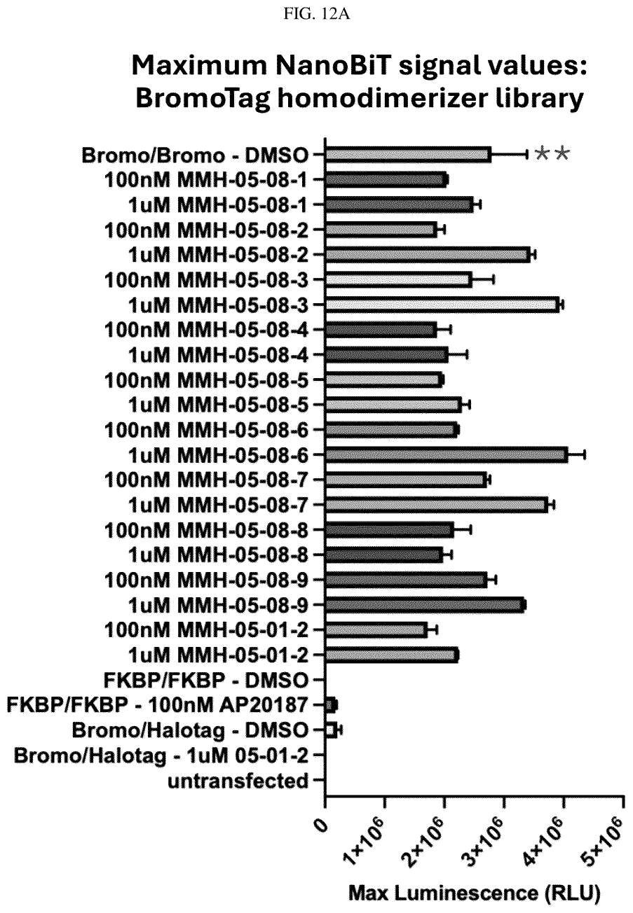

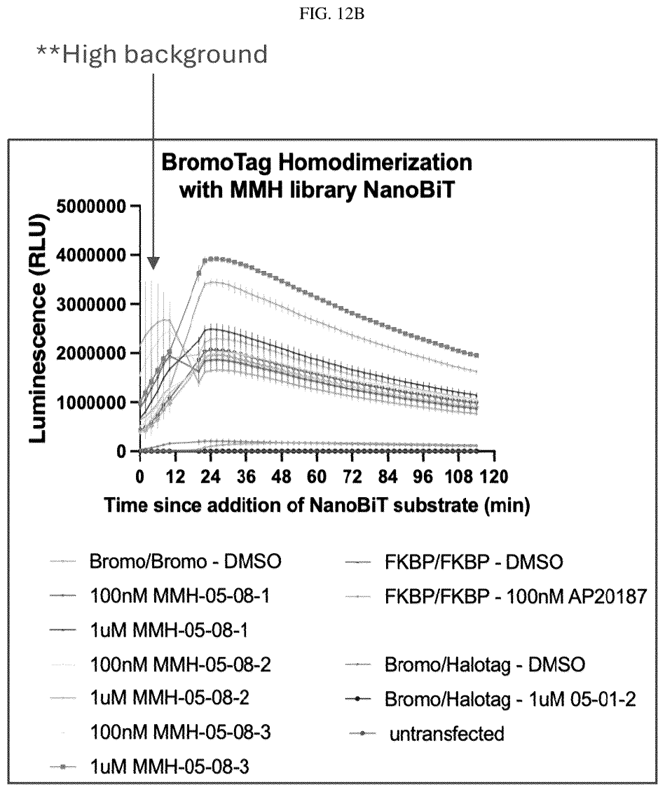

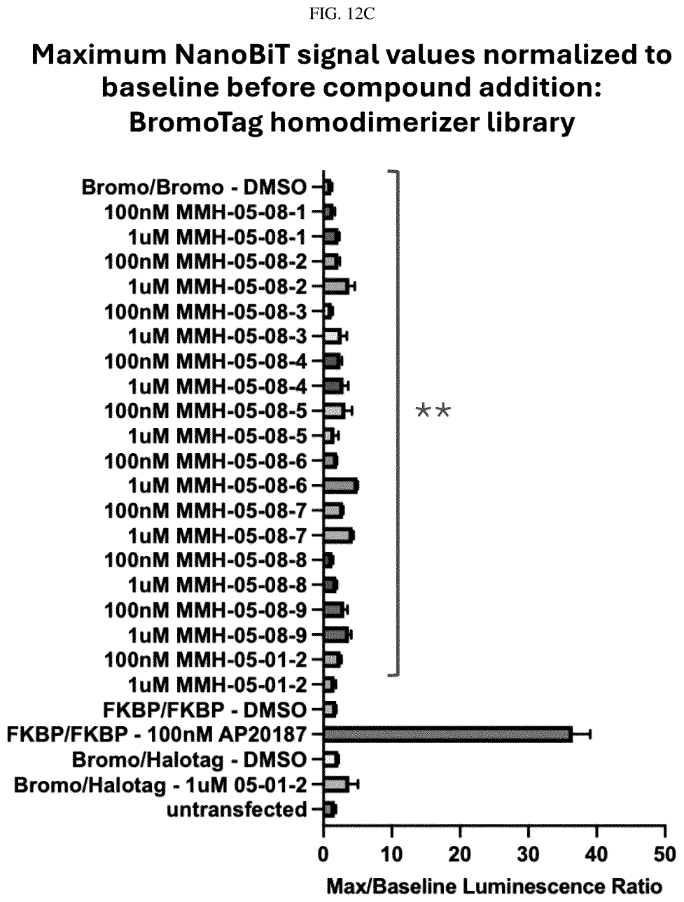

NanoBiT assay. 15k HEK293T cells per well were plated in 92 ul DMEM+10% FBS in opaque 96-well plates and allowed to adhere overnight, approximately 18-24 hours. HEK293T cells were subsequently transfected with pBiT1.1 (LgBiT-tagged) or pBiT2.1 (SmBiT-tagged) FKBP12_{F36V} and/or BromoTag vectors, using TransIT-LT1 transfection reagent (Mirus #MIR2300) and Opti-MEM (Gibco #31985062). After 24 hours, 25 ul Nano-Glo® Live Cell Reagent was added to each well to measure dimerization signal. Using a PHERAstar FSX, baseline luminescence was measured for five or six cycles (approximately eight to ten minutes). Test dimerizer compounds freshly diluted in DMEM+10% FBS were then manually spiked in and induced luminescence was measured continuously for up to two hours. All summary figures were generated with GraphPad Prism. Data for the NanoBit assay is provided in FIGS. 1A-1D, 2A-2B, 3A-3B, 4A-4D, 5, 6A-6B, 7, and 12A-12C.

FIGS. 1A-1D demonstrate that MMH-04-74 induced dramatically higher max dimerization signal than FKBP12_{WT}/FRB-rapamycin (AKA sirolimus) control (boxed) in all LgBiT and SmBiT tag orientations. For this assay, FKBP/BromoTag/FRB were expressed alone, with no fusion protein partners.

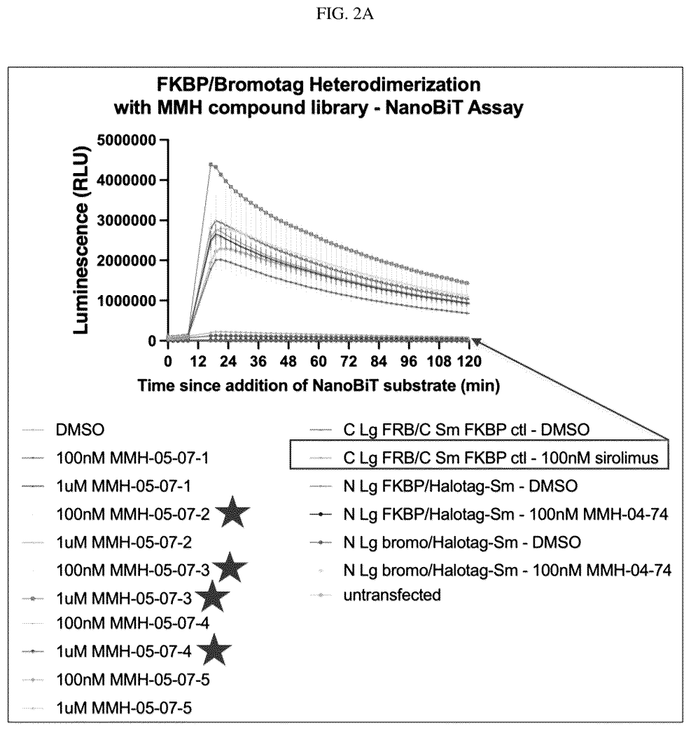

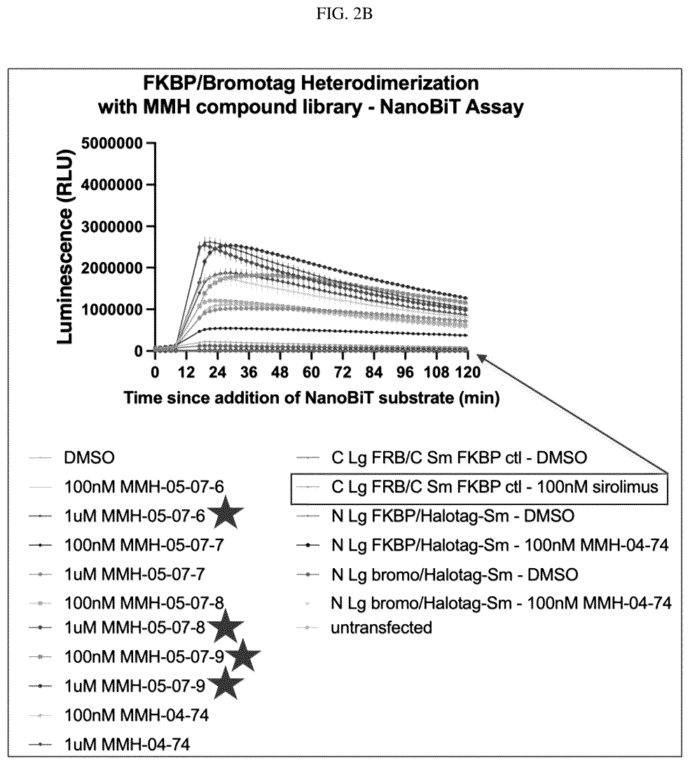

FIGS. 2A-2B demonstrate that all compounds assayed, at both 100 nM and 1 uM, induced dramatically higher max dimerization signal than FKBP12_{WT}/FRB-rapamycin (AKA sirolimus) control (boxed). For this assay, FKBP/BromoTag/FRB were expressed alone, with no fusion protein partners.

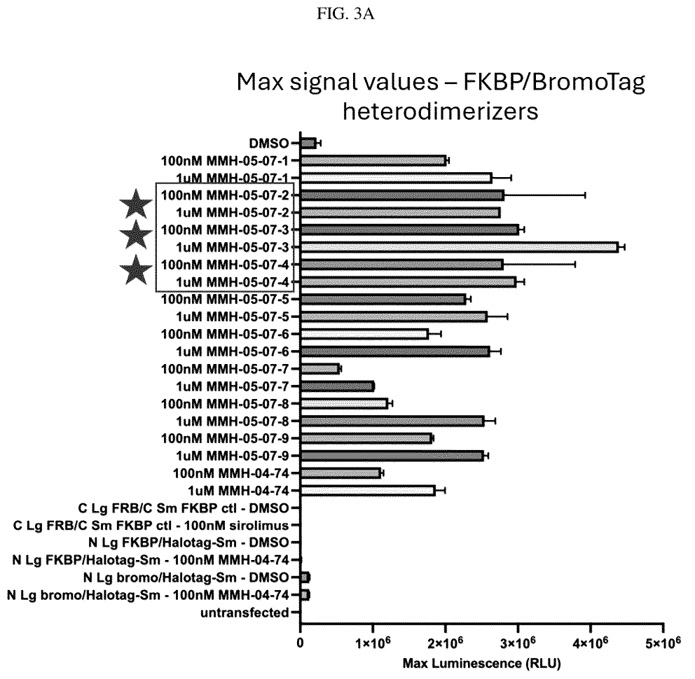

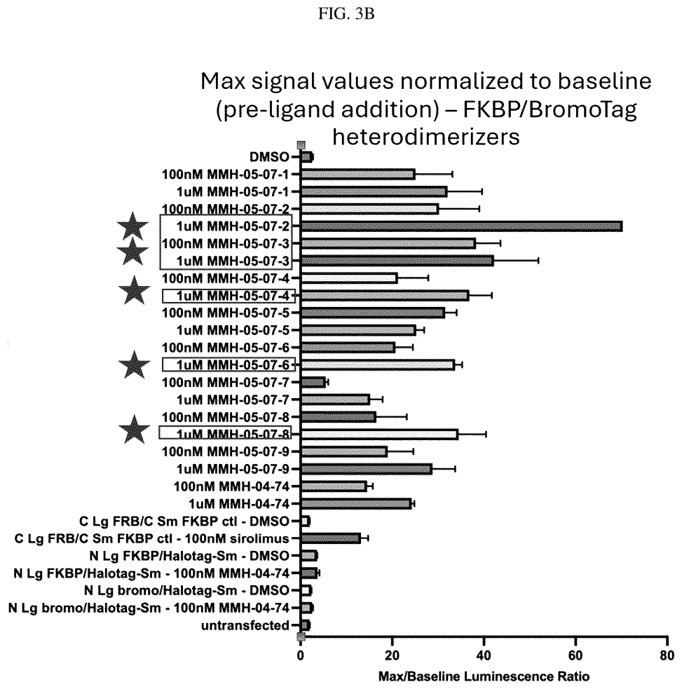

FIGS. 3A-3B demonstrate that all compounds assayed outperformed existing FKBP12_{WT}/FRB-rapamycin (AKA sirolimus) heterodimerization system in max dimerization signal and most outperformed FKBP12_{WT}/FRB system in maximum signal normalized to baseline (pre-compound addition). For this assay, FKBP/BromoTag/FRB were expressed alone, with no fusion protein partners.

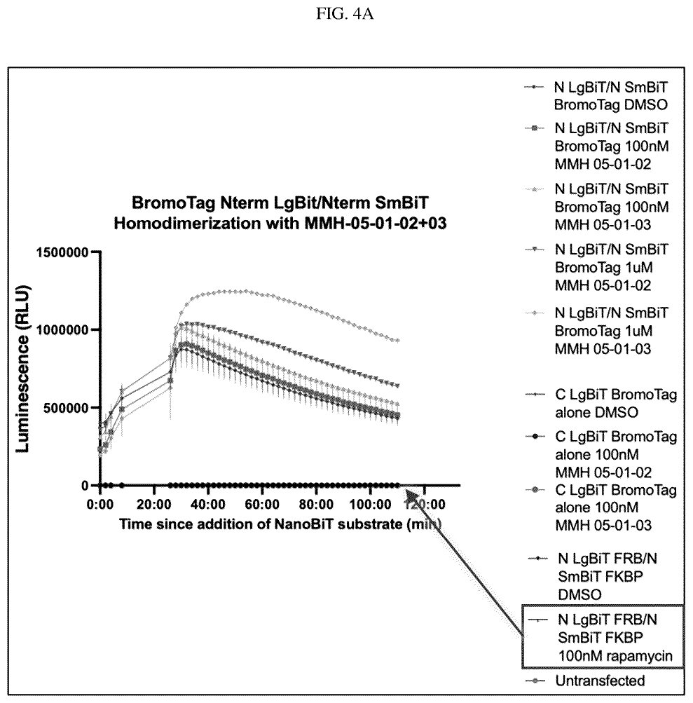

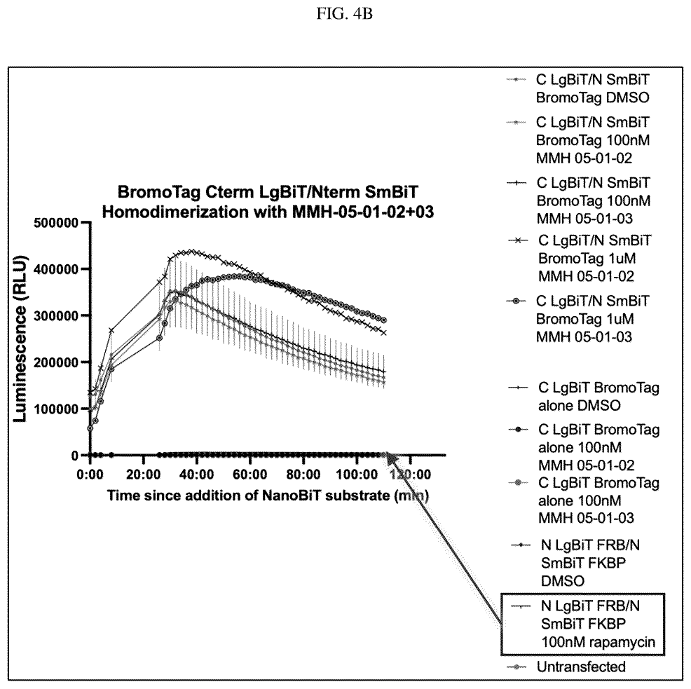

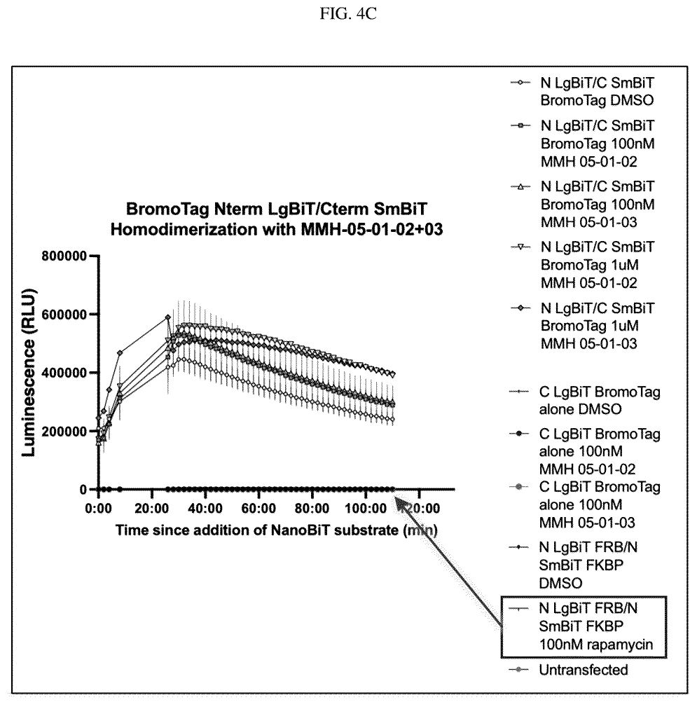

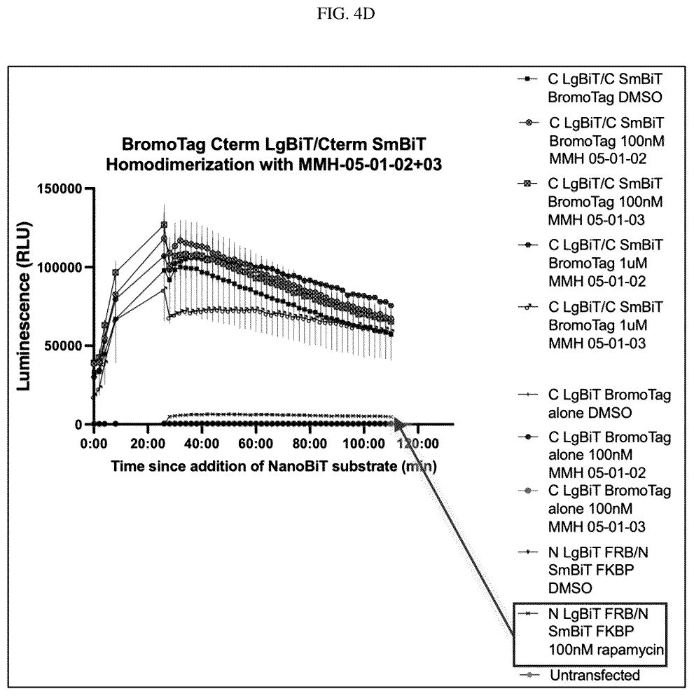

FIGS. 4A-4D demonstrate that, despite high background, both MMH-05-01-02 and MMH-05-01-03 (later renamed MMH-05-08-2) improved max dimerization signal over DMSO and had significantly higher signal than FKBP12_{WT}/FRB-rapamycin control (boxed). For this assay, FKBP/BromoTag/FRB were expressed alone, with no fusion protein partners.

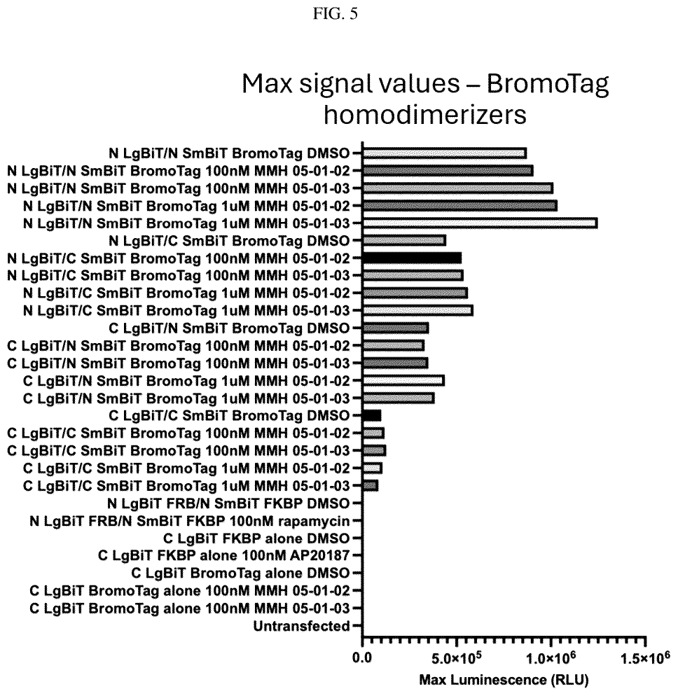

FIG. 5 demonstrates that both MMH-05-01-02 and MMH-05-01-03 (later renamed MMH-05-08-2) outperformed existing FKBP12_{WT}/FRB-rapamycin heterodimerization system in max dimerization signal and in most NanoBiT tag orientations have improved signal over DMSO, despite high background. For this assay, FKBP/BromoTag/FRB were expressed alone, with no fusion protein partners.

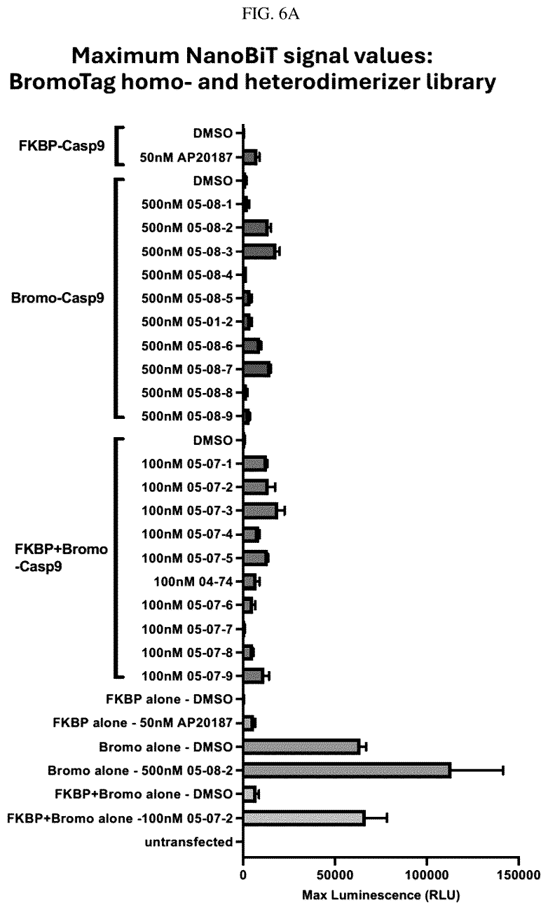

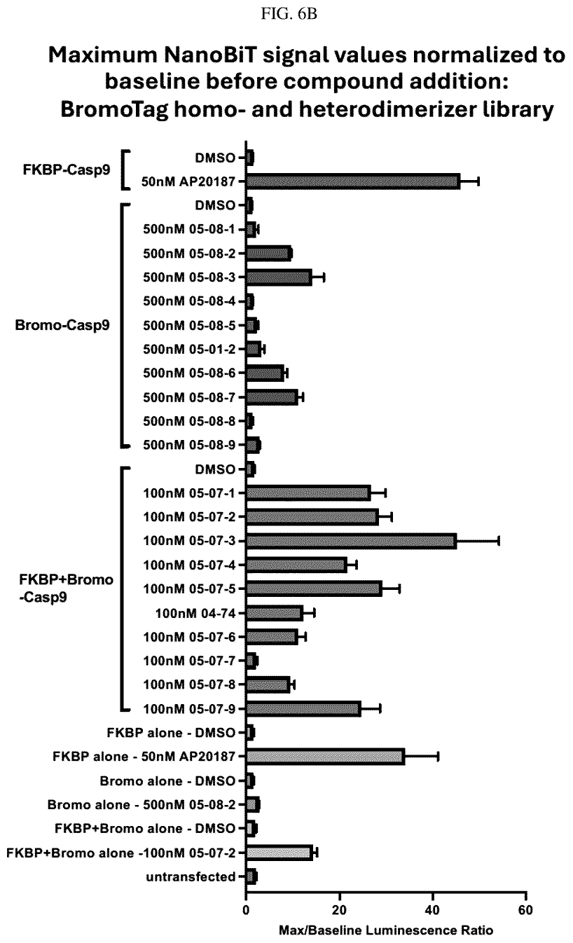

FIGS. 6A and 6B demonstrate that four BromoTag homodimerizers and nine FKBP12_{F36V}/BromoTag heterodimerizers produced robust Casp9 dimerization, as quantified by maximum NanoBiT signal (FIG. 6A) and normalized maximum signal (FIG. 6B). For this assay, FKBP and BromoTag were appended to Casp9 protein.

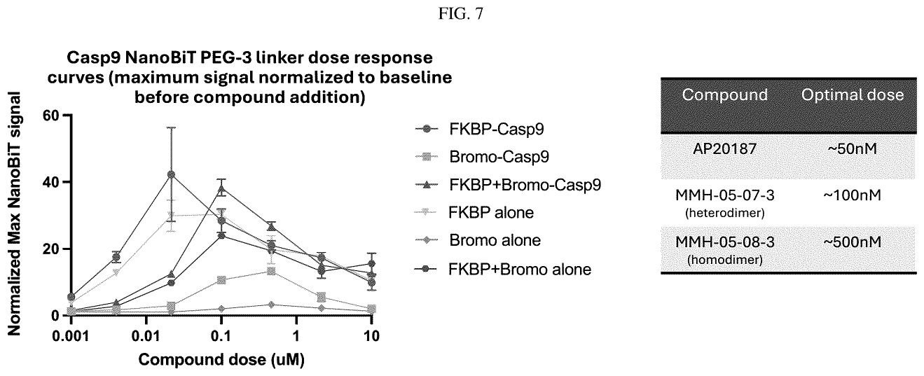

Dosing was optimized for each family of compounds to maximize signal output. Data for the dose optimization studies is provided in FIG. 7. In FIG. 7, doses corresponding to peak signal (after normalization to baseline) are indicated (chart at right side), which all occur in the sub-micromolar range. All compounds display the “hook effect” characteristic of bivalent compounds.

c-KIT phosphorylation Western blotting. HEK293T cells in DMEM+10% FBS were plated at 450K cells per well in 6-well plates and allowed to adhere overnight, about 18-24 hours. The following day, cells were transfected with either c-KIT ICD homodimerization constructs (pLX307 FKBP12_{F36V}-c-KIT ICD, pLX307 BromoTag_{L387A}-c-KIT ICD, or pLX307 2x-BromoTag_{L387A}-c-KIT ICD), or with c-KIT ICD heterodimerization co-expression constructs (pLX307 FKBP12_{F36V}-c-KIT ICD-[P2A+T2A]-BromoTag_{L387A}-c-KIT ICD, pLX307 FKBP12_{F36V}-c-KIT ICD-[P2A+T2A]-2x-BromoTag_{L387A}-c-KIT ICD, or pLX307 FRB_{T1098L}-c-KIT ICD-[P2A+T2A]-FKBP_{WT}-c-KIT ICD) using TransIT-LT1 transfection reagent (Mirus #MIR2300) and Opti-MEM (Gibco #31985062). After 24 hours of construct expression, the transfected cells were divided equally by volume into 12-well plates, allowed to adhere for one hour, and then treated with dimerizer compounds. After treatment, cell pellets were rinsed once with PBS and lysed on ice in RIPA lysis buffer containing a protease inhibitor cocktail (Sigma-Aldrich #P8340), phosphatase inhibitor cocktail 2 (Sigma-Aldrich #P5726), and phosphatase inhibitor cocktail 3 (Sigma-Aldrich #P0044). Lysates were sonicated at 45% amplitude for 4 seconds in a 1 second on/1 second off pattern, followed by centrifugation at 15k rpm at 4° C. The supernatants were collected, and protein concentrations were quantified using a Pierce™ BCA Protein Assay Kit (Thermo Scientific #23225) to ensure equal loading. For immunoblots, the following primary antibodies were used: c-KIT (Cell Signaling #3074, 1:1,000 v/v dilution), phospho-c-KIT (Tyr703) (Cell Signaling #3073, 1:1,000 v/v dilution), and j-actin (Cell Signaling #3700, 1:5,000 v/v dilution). The following secondary antibodies were used: goat anti-Mouse (LI-COR #926-68020, 1:10,000 v/v dilution) and goat anti-Rabbit (LI-COR #926-32211, 1:10,000 v/v dilution). All antibodies were diluted in Intercept® (TBS) Protein-Free Blocking Buffer (LI-COR #927-80001) with 0.2% v/v Tween-20 (Sigma #P1379).

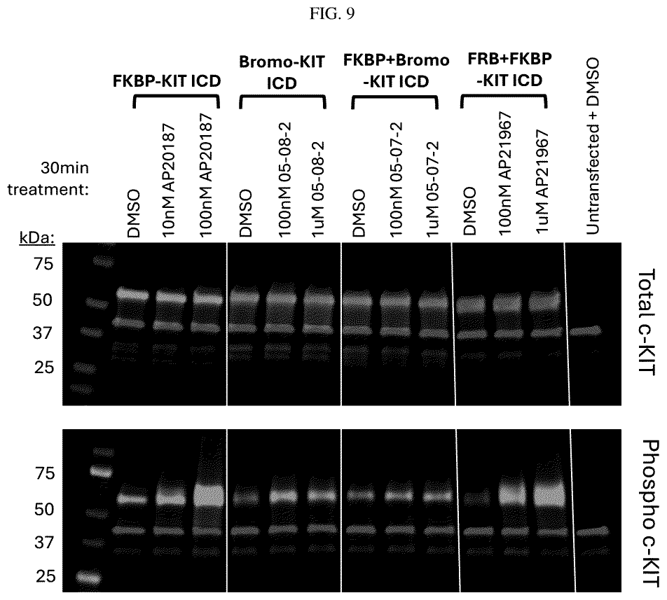

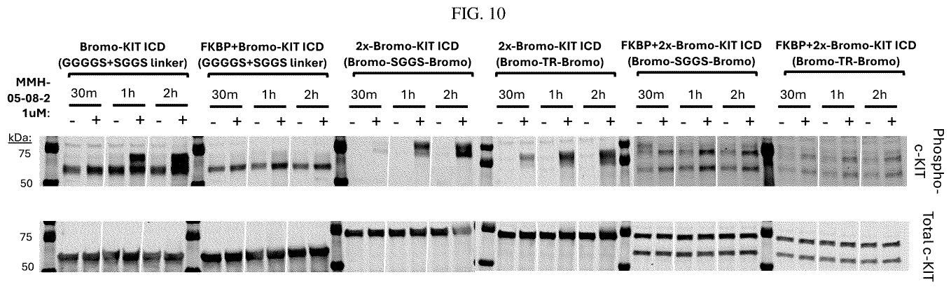

Data for these studies is provided in FIGS. 9 and 10. FIG. 9 provides Western blot depicting c-KIT activation following CID. For the data provided in FIG. 9, HEK293T cells were transfected to express the intracellular domain of c-KIT fused to FKBP, BromoTag, and/or FRB dimerization tags, and subsequently treated with BromoTag dimerizer compounds or AP20187 homodimerizer/AP21967 heterodimerizer control compounds. All test compounds resulted in c-KIT phosphorylation at residue Y703. FIG. 10 provides Western blots depicting c-KIT activation following CID, testing the effect of extending the protein linker between BromoTag and KIT or including multiple BromoTags in tandem. For the data provided in FIG. 10, HEK293T cells were transfected with constructs containing a long, flexible GGGGS+SGGS linker between BromoTag and c-KIT or with constructs containing two copies of BromoTag separated by a SGGS (flexible) or TR (rigid) linker. BromoTag dimerizer compounds resulted in c-KIT phosphorylation at residue Y703 across all transfection groups, particularly at the 1-hour and 2-hour timepoints. All construct modifications produced better dimerizer-induced phosphorylation signal than parent constructs (see FIG. 9). The 2x-BromoTag constructs were particularly efficacious at decreasing background phosphorylation signal while enhancing dimerizer-induced signal.

Caspase-Glo® 3/7 activation assay and Incucyte cell death Imaging. HEK293T cells in DMEM+10% FBS were plated at 400K cells per well in 6-well plates and allowed to adhere overnight before transfection with pHR-FKBP12_{F36V}-Caspase 9 and/or BromoTag_{L387A}-Caspase 9 constructs using TransIT-LT1 transfection reagent (Mirus #MIR2300) and Opti-MEM (Gibco #31985062). After 24 hours of construct expression, the transfected HEK293T cells were replated in either opaque 384-well plates for Caspase-Glo® 3/7 assays or clear-bottom 96-well plates for Incucyte imaging.

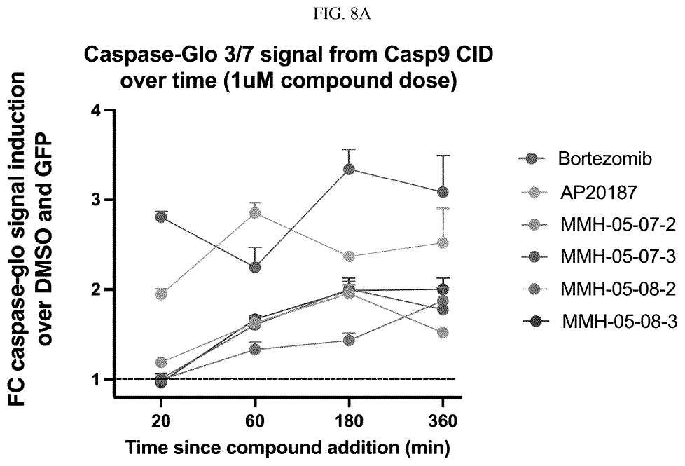

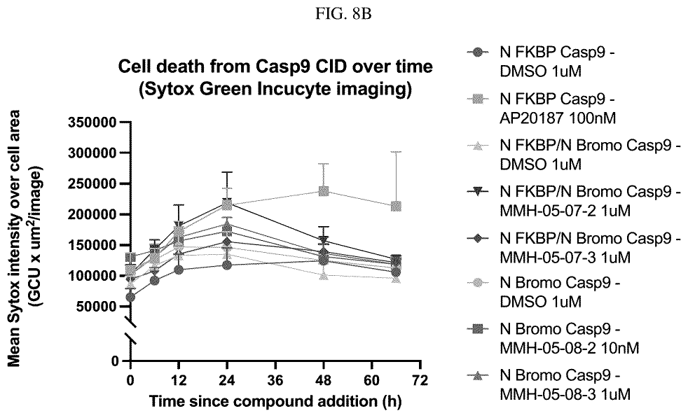

For the Caspase-Glo® 3/7 activation assay, 2,000 cells per well were plated in 15 ul of DMEM+10% FBS. Cells were allowed to adhere for 2 hours before treatment with dimerizer ligand for 20, 60, 180, or 360 minutes. 15 ul of Caspase-Glo® 3/7 Reagent (Promega #G8090) was then added to each well and allowed to equilibrate for 1 hour. Luminescence resulting from Caspase 3/7 enzymatic activity was measured using a PHERAstar FSX. All summary figures were generated with GraphPad Prism. Data is provided in FIG. 8A.

For the Incucyte assay, 4,000 cells per well were plated in 100 ul of DMEM+10% FBS+20 nM Sytox Green dead-cell nucleic acid stain (Invitrogen #57020). Cells were allowed to completely adhere overnight before treatment with dimerizer ligands the following day. Three images per well were taken every 6 hours following compound treatment. After 66 hours, Sytox-positive cell abundance was quantified using built-in Incucyte analysis programs and the statistic ‘mean Sytox intensity over cell area’ (GCU×um²/image). All summary figures were generated with GraphPad Prism. Data is provided in FIG. 8B.

Data for these studies is provided in FIGS. 8A-8B. Activation of Casp9, a pro-apoptotic protein, via chemically-induced dimerization (CID) was quantified using cell death assays. Caspase 3/7 activation (downstream of Casp9 activation) was observed in FKBP- and/or BromoTag-Casp9 transfected HEK293T cells following treatment with dimerizer compounds (see FIG. 8A). Similar results were obtained using an imaging-based cell death assay (see FIG. 8B).

Virus production and stable Ba/F3 cell line generation. For homodimerizer testing, pLX307 FKBP12_{F36V}-c-KIT ICD and pLX307 BromoTag_{L387A}-c-KIT ICD constructs containing N-terminal dimerization tags were used to produce lentivirus. For heterodimerizer testing, pLX307 FKBP12_{F36V}-c-KIT ICD-[P2A+T2A]-BromoTag_{L387A}-c-KIT ICD and pLX307 FRB_{T1098L}-c-KIT ICD-[P2A+T2A]-FKBP_{WT}-c-KIT ICD self-cleaving co-expression constructs were used. To begin lentivirus production, HEK293T lenti+ cells were plated in DMEM+10% FBS at 500K cells per well in 6-well plates and allowed to adhere overnight. Co-transfection with c-KIT ICD constructs, psPAX2 viral packaging plasmid, and pMD2.G viral envelope plasmid using TransIT-LT1 transfection reagent (Mirus #MIR2300) and Opti-MEM (Gibco #31985062) occurred the following day (day 2). Media was changed to DMEM+20% FBS on day 3 to stimulate viral production, and on days 4 and 5 the virus-containing media was harvested, filtered through 0.45 m PES membranes, and pooled.

2 million Ba/F3 cells in RPMI+glutamine+10% FBS+10 ng/mL mIL-3 media per condition were subsequently infected (by centrifugation at 931rcf for 2 hours) with 250 ul of lentivirus containing Sug/mL polybrene infection reagent (Sigma-Aldrich #TR-1003). Outgrowth in antibiotic-free media proceeded for one day before changing to fresh media containing 1.33 ug/mL puromycin (InvivoGen #ant-pr). Puromycin selection occurred for 6 days.

Ba/F3 cellular transformation assay. Ba/F3 cells stably expressing pLX307 FKBP12_{F36V}-c-KIT ICD, pLX307 BromoTag_{L387A}-c-KIT ICD, pLX307 FKBP12_{F36V}-c-KIT ICD-[P2A+T2A]-BromoTag_{L387A}-c-KIT ICD or pLX307 FRB_{T1098L}-c-KIT ICD-[P2A+T2A]-FKBP_{WT}-c-KIT ICD constructs were washed twice with PBS, resuspended in either RPMI+glutamine+10% FBS+10 ng/mL mIL-3 or base RMPI+glutamine+10% FBS without mIL-3, and plated at 1 million cells/mL in 12-well plates. After 1 hour, cells were treated with dimerizer compounds. Counts of viable cells were subsequently taken every 2 days with a Luna II automated cell counter.

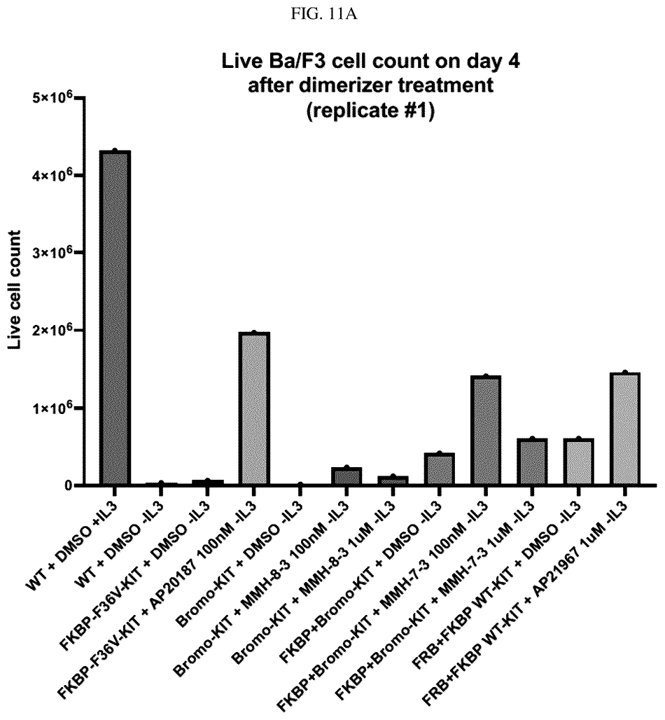

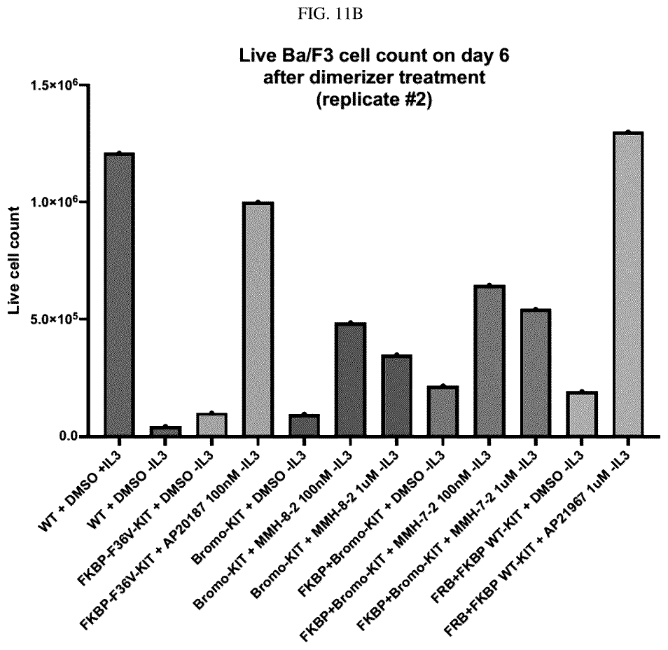

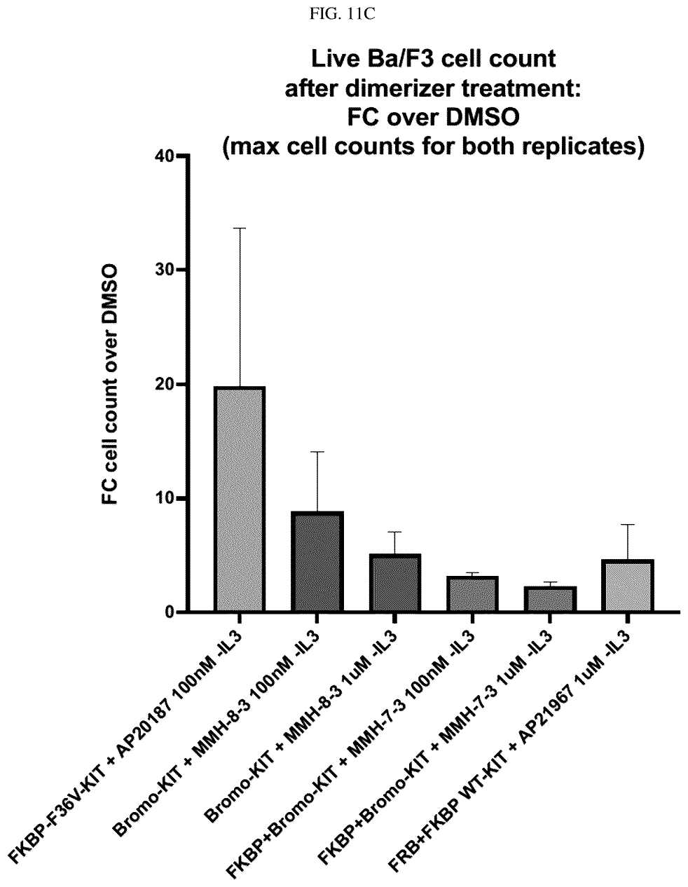

Data for this assay is provided in FIGS. 11A-11C. Ba/F3 cells cultured in media lacking mIL-3 only survive in the presence of oncogenic stimuli. In Ba/F3 cells stably expressing oncogenic c-KIT tagged with FKBP12, BromoTag, and/or FRB, c-KIT activation via CID caused growth in mIL-3-deprived media. Raw cell counts at the timepoint with maximum observed cell survival are shown for two replicate experiments (see FIGS. 11A and 11B). An average of both replicates, normalized to DMSO-treated cell counts for each Ba/F3 cell line is depicted in FIG. 11C.

1. A compound of formula (I): 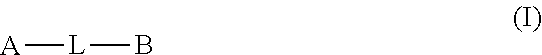
or a salt thereof, wherein:
A is a moiety that binds to BRD4 BD2-domain;
L is a linker; and
B is selected from: (i) a moiety that binds to BRD4 BD2-domain; and (ii) a moiety that binds to FKBP12.

2. The compound of claim 1, or a salt thereof, wherein A is:

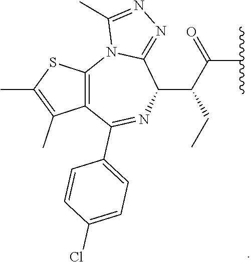 [SMILES: *C(=O)[C@H](CC)[C@@H]1N=C(c2ccc(Cl)cc2)c2c(sc(C)c2C)-n2c(C)nnc21]

3. The compound of claim 1, or a salt thereof, wherein B is a moiety that binds to BRD4 BD2-domain.

4. The compound of claim 3, wherein B is:

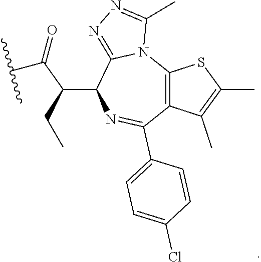 [SMILES: *C(=O)[C@H](CC)[C@@H]1N=C(c2ccc(Cl)cc2)c2c(sc(C)c2C)-n2c(C)nnc21]

5. The compound of claim 1, or a salt thereof, wherein B is a moiety that binds to FKBP12.

6. The compound of claim 5, wherein B is:

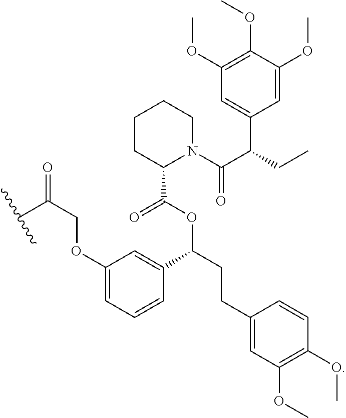 [SMILES: *C(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

7. The compound of claim 1, or a salt thereof, wherein the compound is a compound of formula (Ia):

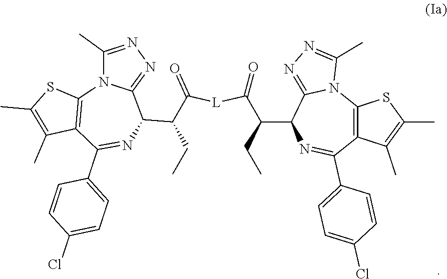

8. The compound of claim 1, or a salt thereof, wherein the compound is a compounds of formula (Ib):

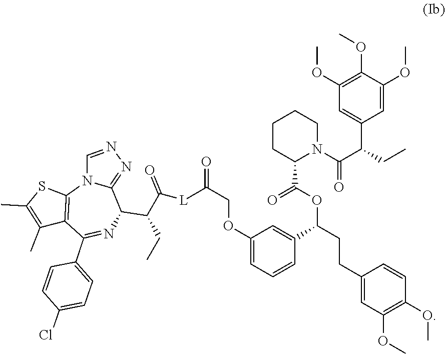

9. The compound of claim 1, or a salt thereof, wherein L is a direct bond or comprises any combination of —CH2-, —CH═CH—, —C≡C—, —O—, —NR′—, —BR′—, —S—, —C(O)—, —C(NR′)—, —S(O)—, —S(O)2-, arylene, heteroarylene, cycloalkylene, and heterocyclylene moieties, wherein the arylene, heteroarylene, cycloalkylene, and heterocyclylene moieties are independently unsubstituted or substituted with 1, 2, or 3 substituents.

10. The compound of claim 1, or a salt thereof, wherein L is a direct bond or comprises any combination of —CH2-, —O—, —NH—, and heterocyclylene moieties.

11. The compound of claim 1, or a salt thereof, wherein L comprises any combination of the following moieties: 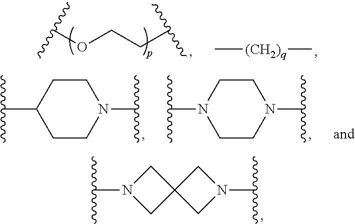
wherein p is 1, 2, 3, or 4; and q is 1, 2, 3, 4, 5, 6, 7, 8, 9, 10, 11, or 12.

12. The compound of claim 1, or a salt thereof, wherein L is:

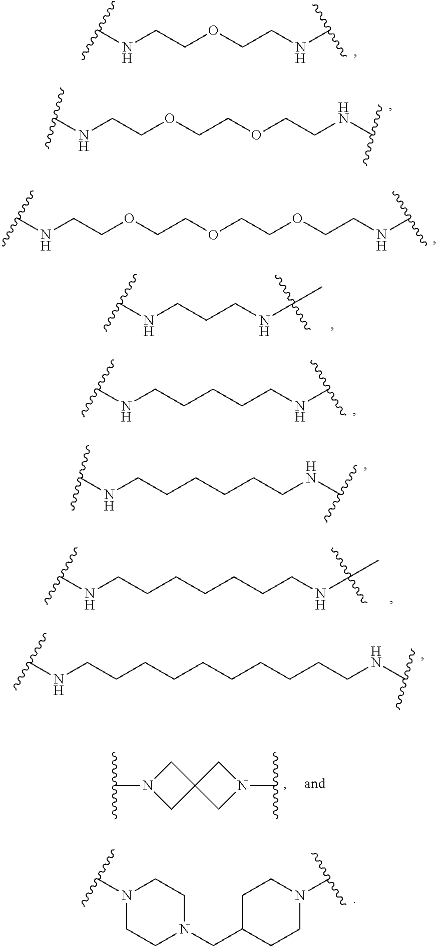

13. The compound of claim 1, wherein the compound is selected from:

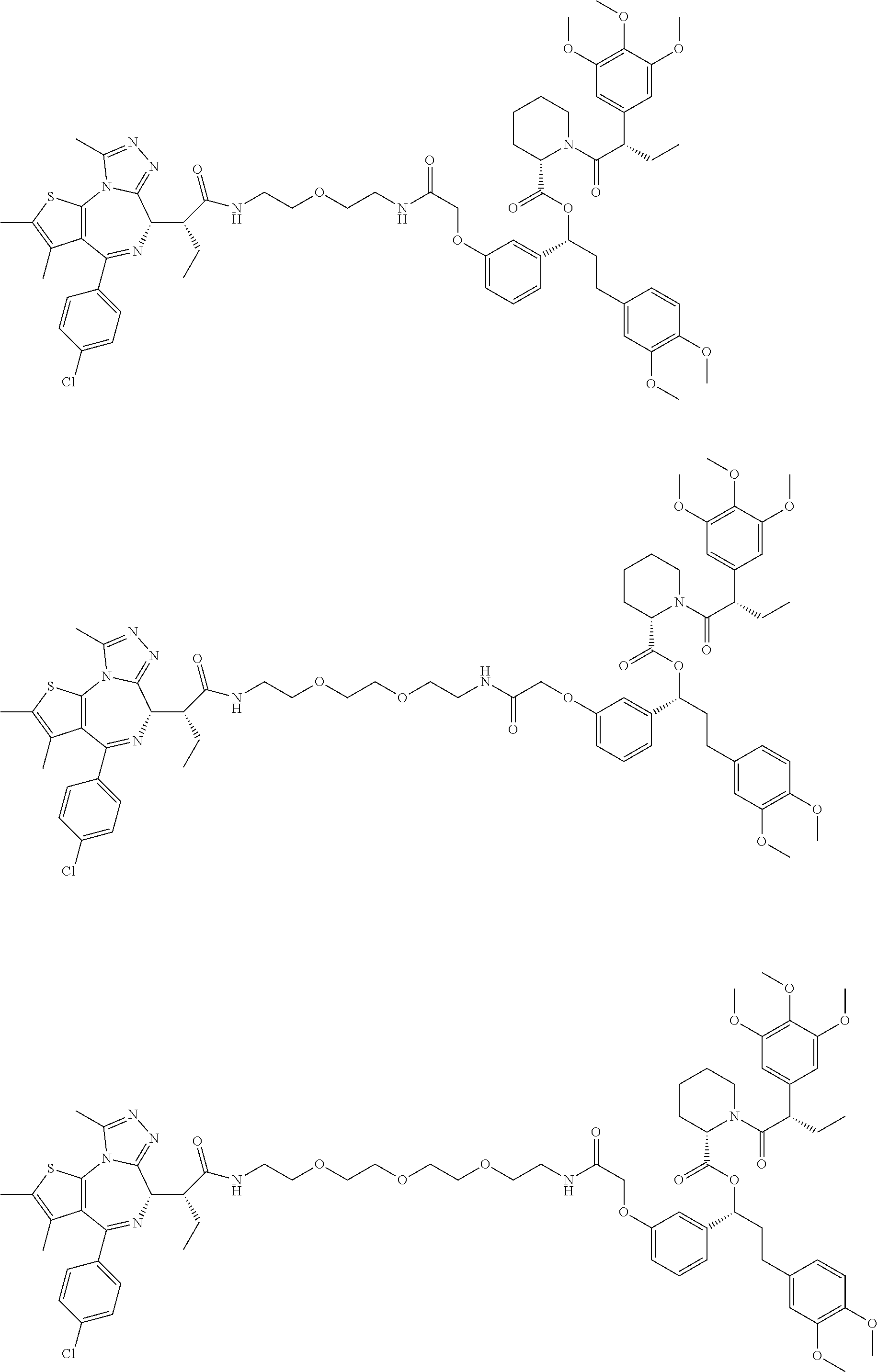

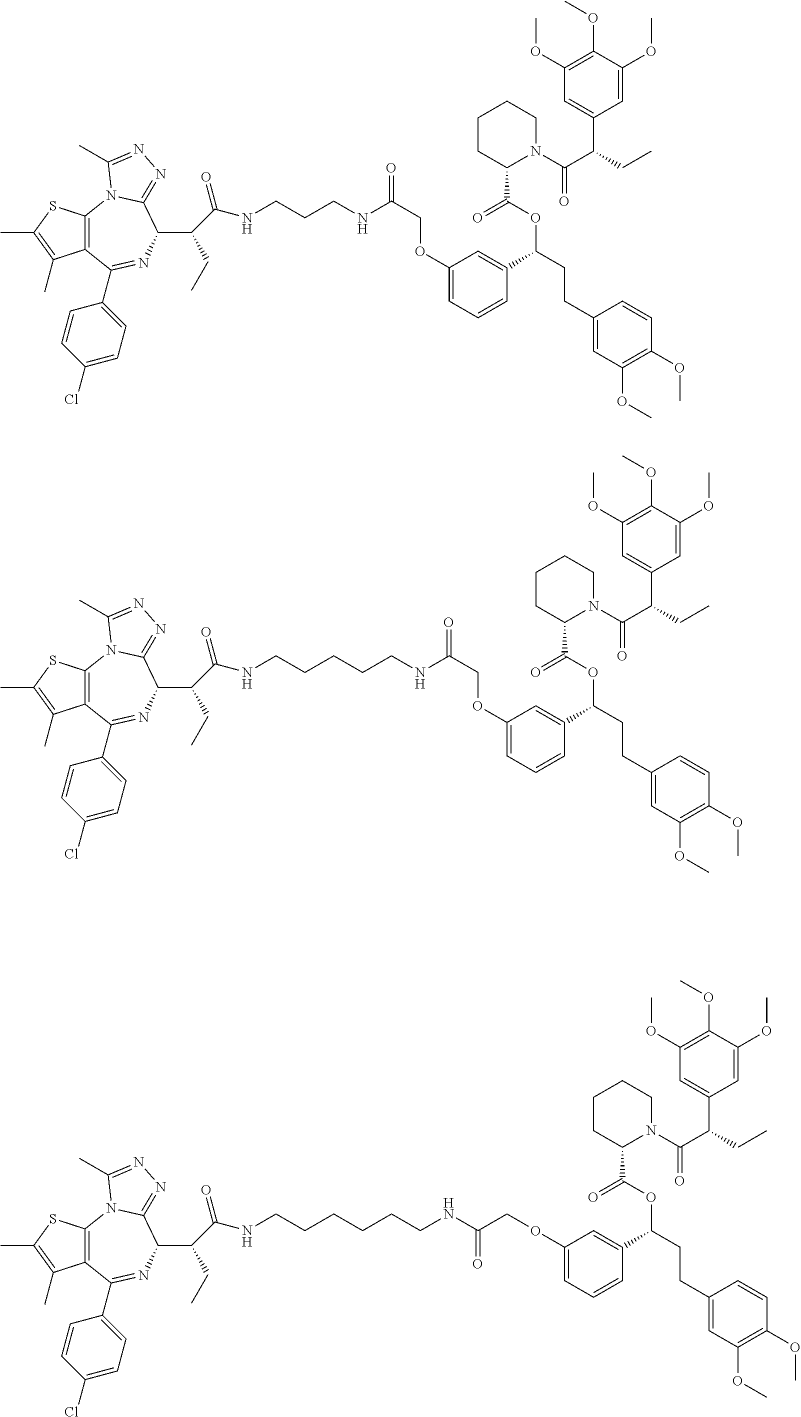 [SMILES: CC[C@H](C(=O)N1CCCC[C@H]1C(=O)O[C@H](CCc1ccc(OC)c(OC)c1)c1cccc(OCC(=O)NCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1]

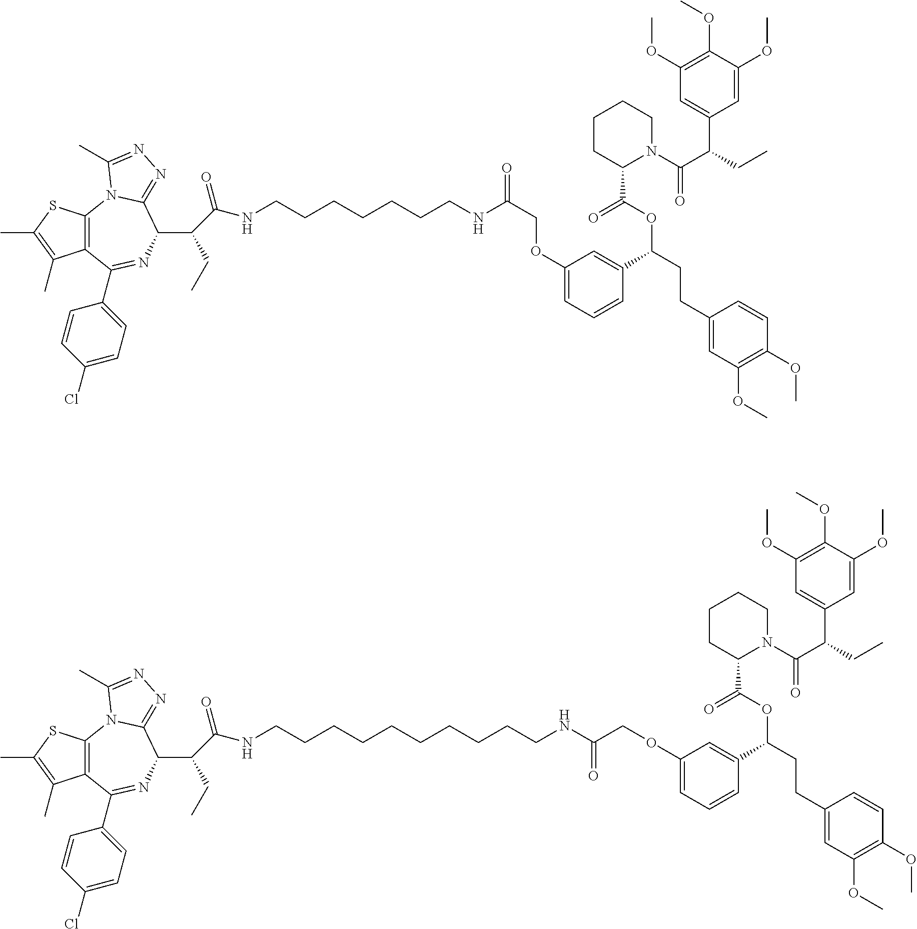 [SMILES: CC[C@H](C(=O)N1CCCC[C@H]1C(=O)O[C@H](CCc1ccc(OC)c(OC)c1)c1cccc(OCC(=O)NCCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCNC(=O)[C@H](CC)[C@@H]2N=C(c3ccc(Cl)cc3)c3c(sc(C)c3C)-n3c(C)nnc32)c1)c1cc(OC)c(OC)c(OC)c1]

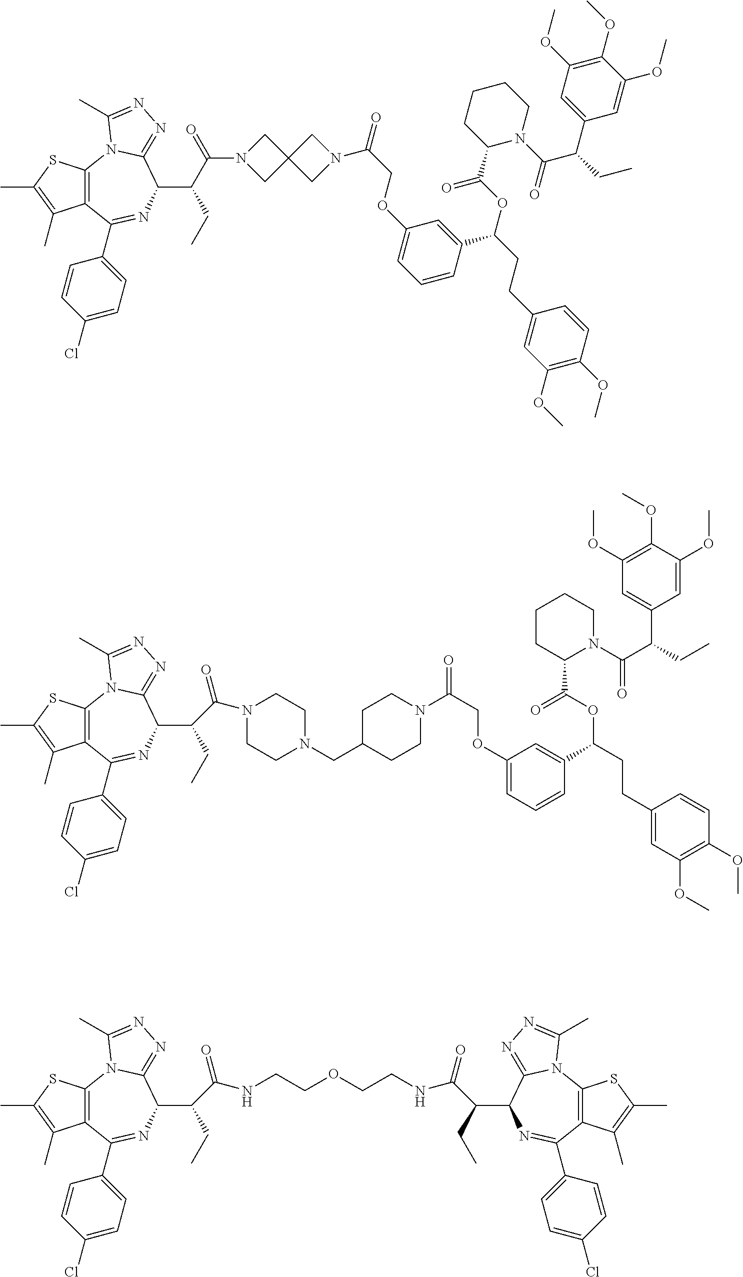

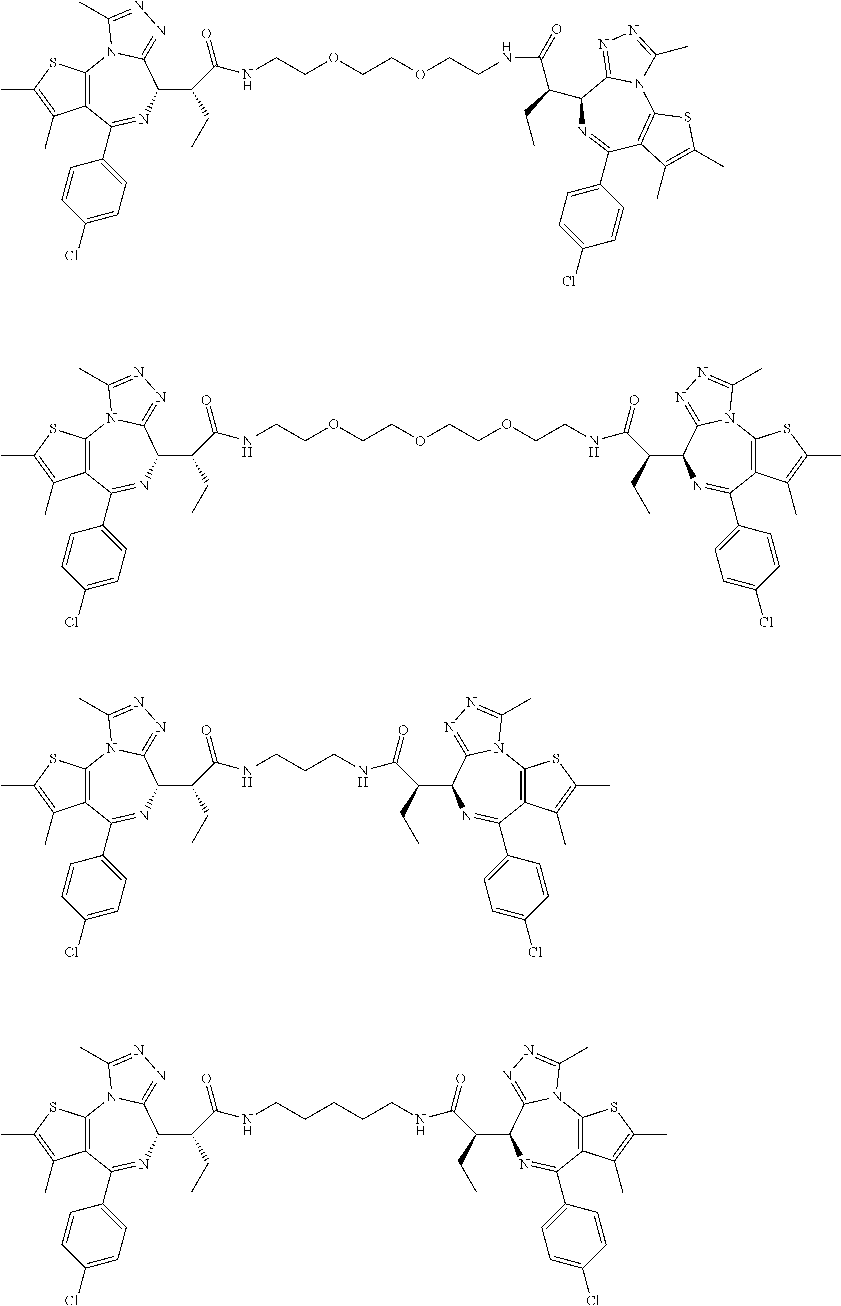

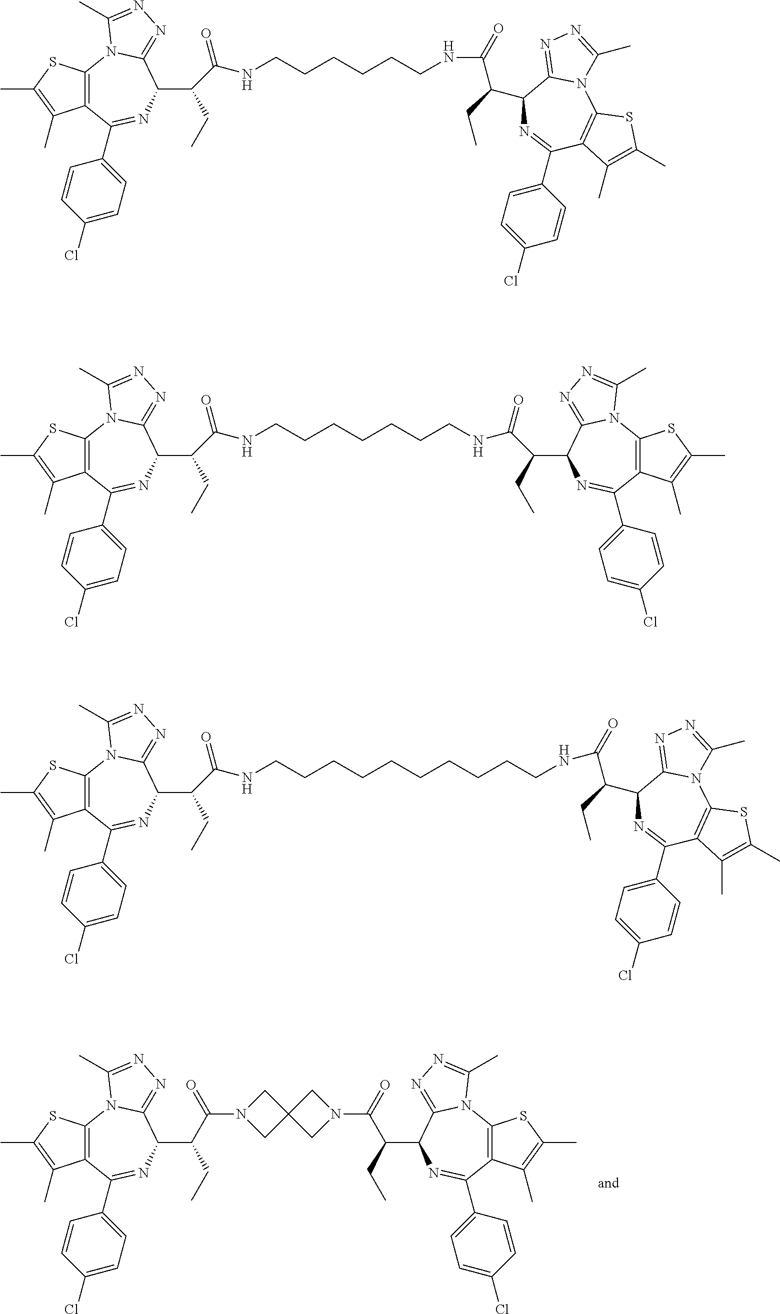

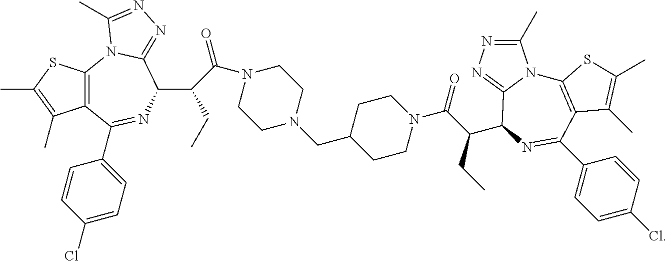 [SMILES: CC[C@@H](C(=O)N1CCC(CN2CCN(C(=O)[C@H](CC)[C@@H]3N=C(c4ccc(Cl)cc4)c4c(sc(C)c4C)-n4c(C)nnc43)CC2)CC1)[C@@H]1N=C(c2ccc(Cl)cc2)c2c(sc(C)c2C)-n2c(C)nnc21]

14. A method of inducing dimerization between two Brd4 BD2 domains in a sample, comprising contacting the sample with a compound of claim 3.

15. A method of inducing dimerization between a Brd4 BD2 domain and FKBP12 in a sample, comprising contacting the sample with a compound of claim 5.

16. A system or kit comprising a compound of claim 1.
